(19) United States
(12) Patent Application Publication
(10) Pub. No.: US 2025/0276035 A1
(43) Pub. Date: Sep. 4, 2025

(54) ANTIBACTERIAL POLYPEPTIDES

(71) Applicants: Novalysin AB, Lund (SE); Universiteit Gent, Gent (BE)

(72) Inventors: Adel Elsayed Attia Abouhmad, Lund (SE); Rajni Hatti Kaul, Lund (SE); Yves Briers, Nederokkerzeel (BE); Dennis Grimon, Gent (BE); Mats Clarsund, Gent (SE); Tarek Dishisha, Lund (SE)

(21) Appl. No.: 18/549,364

(22) PCT Filed: Mar. 9, 2022
(86) PCT No.: PCT/EP2022/056059

§ 371 Date: Sep. 7, 2023

(30) Foreign Application Priority Data
Mar. 9, 2021 (SE) 2150270-3

(51) Int. Cl. A61K 38/16; A61K 38/46; A61K 45/06; A61P 31/06; C07K 14/35; C12N 9/18

(52) U.S. Cl. CPC A61K 38/164; A61K 38/465; A61K 45/06; A61P 31/06; C07K 14/35; C12N 9/18; C12Y 301/01013; C07K 2319/00

(57) ABSTRACT

The present invention relates to antimicrobial peptides comprised of a first domain with activity specific to a peptidoglycan or component thereof; a second domain with activity specific to an ester linkage; and a third domain with membrane permeabilising activity. The invention also relates to the use of such peptides in medicine, for example for treating mycobacterial infection.

### FIELD

The present invention is in the field of antimicrobial peptides, in particular those for use against mycobacteria.

### BACKGROUND

Mycobacterium tuberculosis (Mtb), an acid-fast microorganism, is the causative agent of tuberculosis (TB), which is considered a health threat due to the highly emerged resistance rates (Muller et al., 2013). TB is considered one of the leading causes of death worldwide with 10 million patients in 2017 (WHO 2018) with half million new multidrug resistant TB cases. Despite the existence of curative chemotherapy, the emergence of the alarming context of multidrug-resistant (MDR), extremely drug-resistant (XDR), and totally drug-resistant (TDR) TB and the lack of effective therapies has increased the demand for developing new and innovative antimycobacterials (Marrakchi et al., 2014; Mitnick et al., 2009).

The major characteristic feature of mycobacteria is their unique cell wall structure with up to 60% lipid content compared to 5-10% for Gram-positive and Gram-negative bacteria (Neyrolles & Guilhot, 2011). The cell wall of Mtb consists of an inner peptidoglycan layer that is covalently linked to arabinogalactan, which is esterified with mycolic acids (MA). MA are long chain (C₆₀-C₉₀), α-branched, β-hydroxy fatty acids containing cyclopropane rings, double bonds, and oxygenated groups, according to the species and genera (Watanabe et al., 2001). The specific composition of the MA is dependent on the Mycobacterium species including short saturated a, C₂₀₋₂₅, and a longer meromycolate chain, the β-hydroxy branch C₆₀, comprising double bonds, cyclopropane rings and oxygenated groups (Payne et al., 2009).

MA are found in two forms: unbound and bounded. The first is: unbound which are glycolipid esters of trehalose forming trehalose monomycolate (TMM) and trehalose dimycolate (TDM; also called the cord factor) which has a key role in mycobacterial pathogenesis (Bhamidi et al., 2008; Brennan, 2003). The second form of MA is bounded via ester linkage with the terminal pentaarabinofuranosyl units of arabinogalactan (AG), the polysaccharide that together with peptidoglycan, forms the insoluble cell wall skeleton (Brennan & Nikaido, 1995; Daffe, 2008; Daffe, 1996; Hoffmann et al., 2008; Mcneil et al., 1990; Niederweis, 2008). Both forms of MA impart impermeability to the cell envelope and participate in the two leaflets of the mycobacterial outer membrane, the mycomembrane (Sani et al., 2010; Zuber et al., 2008).

The mycomembrane imparts hydrophobicity that results in decreased permeability to nutrients and antimycobacterials making TB difficult to treat (Jarlier & Nikaido, 1990). It is also essential for cell viability, hence the target of antituberculosis (anti-TB) drugs (Vilcheze & Jacobs, 2007). Due to the unique structure of the mycomembrane envelope it is considered a key target for novel antimycobacterials. Tuberculosis drugs target various aspects of Mycobacterium tuberculosis biology, including inhibition of cell wall synthesis, protein synthesis, or nucleic acid synthesis. For some drugs, the mechanisms of action have not been fully identified. The major action of anti-TB drugs is via affecting lipids, glycolysis, nucleic acid synthesis and inhibition of mycolic acid biosynthesis. Anti-TB drugs target the energy system of cells and/or inhibit synthesis of cell wall components. They act intracellularly, and so need to be internalised/trafficked into the target cells. Accordingly, anti-TB drugs are typically ineffective on dormant/latent cells.

The use of endolysins as antibacterial enzymes is increasingly considered for the treatment of bacterial infections. Endolysins are peptidoglycan degrading enzymes produced by phages at the end of the lytic replication cycle. They degrade the cell wall of the infected cells, which eventually results in lysis and dispersion of the viral progeny. The natural function of these endolysins can be exploited to use them as powerful enzyme-based antibiotics, also coined ‘enzybiotics’. Exogenous application of recombinant endolysins to Gram-positive bacteria rapidly induces osmotic lysis and consequent cell death.

In the last two decades, endolysins have proven their efficacy in different models of infection in animals (Nelson et al., 2012) and bacterial contamination in food (Schmelcher et al., 2012). A key feature of endolysins is their modularity and the opportunities that emerge thereof to customize properties of endolysins such as specificity, activity, stability and solubility (Gerstmans et al., 2018). Initially, endolysins were only explored for Gram-positive pathogens as their thick peptidoglycan layer is immediately accessible from the outside. However, protein engineering is being applied to expand the antibacterial spectrum of endolysins to Gram-negative bacteria. Fusion proteins of a single, specific endolysin (KZ144 from Pseudomonas aeruginosa bacteriophage varphiKZ) acting against the peptidoglycan layer of Gram-negatives and a selected outer membrane permeabilizing peptide kill Gram-negative bacteria through local destabilization of the outer membrane, followed by passage of the fusion protein across the outer membrane and degradation of the peptidoglycan layer. This results in immediate cell death through osmotic lysis (Briers et al., 2014). These fusion proteins are called ‘Artilysins’. In addition, a similar protein engineering strategy has been applied to endolysins against Gram-positive pathogens, resulting in engineered endolysins that kill faster, with higher activity (+2 log) and potency.

The complex nature of the mycobacterial cell wall, comprising peptidoglycan, arabinogalactan and mycolic acid layers (FIG. 1), poses a major obstacle for antibiotics in general and has hindered the expansion of enzybiotics toward Mtb until today.

Mycobacteria can be infected by several viruses known as mycobacteriophages that face the same challenge of the unusual structure of the cell wall and are therefore equipped with two different and separate types of enzymes to lyse the mycobacterial cell wall: (1) LysA hydrolyses the peptidoglycan layer (peptidoglycan hydrolase enzymes, also termed endolysins), and (2) LysB cleaves the ester linkage of mycolic acids to the arabinogalactan layer (mycolyl arabinogalactan esterase enzymes that are not typically considered as conventional endolysins, but can instead be categorized as enzymes with lipolytic activity). Although these enzymes are encoded in a lysis cassette in mycobacteriophages, arranged as protein coding gene segments in tandem, they are released as separate proteins and do not exist in nature as fusion proteins.

LysB-D29 enzyme (without any antimicrobial peptide present) showed synergistic effect with anti-TB drugs; Rifampicin (also known as rifampin), Ethambutol and Isoniazid in subminimal concentrations (concentrations which inhibited the growth, but did not show any bactericidal effect) against M. smegmatis and Mycobacterium bovis bacillus Calmette-Guerin (BCG) (Sharma, 2017). Similarly, in comparison with Sharma, WO 2017/023680 A1 used peptides comprised of LysB only, in the context of treating acne.

However, recent evidence has highlighted that LysB peptides alone are insufficient to have antimycobacterial activity (Abouhmad et al, 2020), even when used in combination with anti-TB drugs. Instead, combination with outer membrane permeabilisers, such as colistin and protamine, were required to observe antimycobacterial properties, potentially due to the mycobacterial membrane being highly hydrophobic and thick.

In other studies, two types of peptides are exemplified (Miller, 2015), each of which consist of: (i) an antimicrobial peptide (AMP), LysA and a protein transduction domain (PTD); or (ii) an AMP, LysB and a PTD. However, these fusions of antimicrobial peptide with either LysA or LysB individually did not exert antimycobacterial activity (Miller, 2015). When these peptides were combined as a mixture post expression and purification, antimycobacterial activity was detected. In this study, Miller was limited to the use of a TrxA tag to enhance the solubility of their peptides.

There remains a need for improved peptides which are capable of disrupting cell envelopes, such as those of mycobacteria, in order to exert an antimicrobial effect.

### BRIEF SUMMARY OF THE INVENTION

As discussed, the peptides known in the art do not comprise all the necessary activities within a single peptide due to various complications, such as steric hindrance of multiple units comprised in a fusion peptide. The polypeptides provided herein are comprised of all necessary activities within a single polypeptide while retaining the necessary function of antimicrobial (e.g. antimycobacterial) activity.

The present invention, for the first time, provides peptides comprising at least three domains, as follows: (i) a domain with activity specific to a peptidoglycan or component thereof; (ii) a domain with activity specific to an ester linkage; and (iii) a domain with membrane permeabilising, destabilizing and/or disrupting activity. Such peptides are capable of targeting complex cell envelopes comprised of multiple components, as shown in FIG. 1.

A benefit of peptides of the present invention over antimicrobials (such as anti-TB drugs) is that the peptides are able to act against dormant/latent cells. A further benefit of the peptides is their increased specificity and selectivity to bacteria comprised of the aforementioned complex cell walls, for example mycobacteria. Another benefit is that the fusion peptides (comprising AMP, LysA and LysB) have surprisingly a faster expression rate compared with AMP-LysA and AMP-LysB; and improved inhibitory activity compared with a mixture of corresponding AMP-LysA and AMP-LysB mixtures.

The inventors surprisingly found that all three domains/functions could be provided in a single peptide.

The peptides of the present invention comprise activity specific to a peptidoglycan or component thereof, which enables them to effectively target bacteria with cell walls further comprising a peptidoglycan layer. The peptides also comprise a membrane permeabilising, destabilizing and/or disrupting domain (such as an antimicrobial peptide (AMP)) to further enhance their antibacterial activity. Additionally, in one embodiment, singular peptides of the present invention are capable of killing their target bacteria, which contrasts to peptides of the prior art which require the combination of multiple different peptides to kill target bacteria.

### DETAILED DESCRIPTION OF THE INVENTION

The present invention relates to a peptide comprising three domains:
(i) a domain with activity specific to a peptidoglycan or component thereof;
(ii) a domain with activity specific to an ester linkage; and
(iii) a domain with membrane permeabilising activity.

A “component thereof” may also be referred to as a “portion thereof” herein. These domains may be referred to as a first, second or third domain, respectively. However, a first domain is not to be construed as limited to activity specific to a peptidoglycan or component thereof. For example, the first domain may have activity specific to an ester linkage and/or membrane permeabilising activity. However, the peptides of the invention are at least capable of all three activities as listed above. Additionally, the first, second and third domains can be present in any order in the peptide.

Accordingly, a first aspect of the invention is a peptide comprising a first domain with activity specific to a peptidoglycan or component thereof; a second domain with activity specific to an ester linkage; and a third domain with membrane permeabilising activity.

By “activity specific” we include the meaning that the domain is capable of specifically binding or associating with a second entity (i.e. a target), and/or that the domain enacts a change (for example, the breakdown of the target entity) on a second entity. For example, a domain with activity specific to peptidoglycan would include a domain that induces peptidoglycan hydrolase activity.

Accordingly, in one embodiment a first aspect of the invention is a peptide comprising:
a first domain that is:
   a) capable of specifically binding to a peptidoglycan or component thereof; and/or
   b) capable of specifically associating with a peptidoglycan or component thereof; and/or
   c) capable of enacting a change in a peptidoglycan or component thereof;
and a second domain that is:
   a) capable of specifically binding to an ester linkage; and/or
   b) capable of specifically associating with an ester linkage; and/or
   c) capable of enacting a change in an ester linkage;
and a third domain with membrane permeabilising, destabilizing and/or disrupting activity.

A “peptide” or “polypeptide” is used herein interchangeably in its broadest sense to refer to a compound of two or more subunit amino acids, amino acid analogs, or other peptidomimetics. The term “peptide” thus includes short peptide sequences and also longer polypeptides and proteins, including variants and fusions thereof. As used herein, the term “amino acid” refers to either natural and/or unnatural or synthetic amino acids, including both D or L optical isomers, and amino acid analogs and peptidomimetics.

It will be appreciated by persons skilled in the art that the term “amino acid”, as used herein, includes the standard twenty genetically-encoded amino acids and their corresponding stereoisomers in the ‘d’ form (as compared to the natural ‘I’ form), omega-amino acids other naturally-occurring amino acids, unconventional amino acids (e.g., α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both I-alanine and d-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

A domain may be a “binding domain”, which includes the meaning of any peptide sequence that is capable of binding or associating with a second entity. In the case of the first domain, the domain is able to bind specifically to a peptidoglycan or component thereof; in the case of the second domain, the domain is able to bind specifically to an ester linkage and/or to a peptidoglycan or component thereof; and in the case of the third domain, the domain is able to bind specifically to a cell membrane or component thereof (for example, glycolipids (such as Lipoarabinomannan, TDM, TMM), mycolic acids, phospholipids, etc), such as an outer and/or inner leaflet component of a cell membrane. For example, the third domain may be able to bind to glycolipids and/or phospholipids and/or mycolic acids exposed on a cell membrane.

By “bind specifically” we include the meaning that the domain (or the peptide comprising at least one domain) binds to its target in a manner that can be distinguished from binding to non-target domains (i.e. off-targets). For example, a domain that binds specifically may refer to a domain that binds with higher specificity for the intended target compared with that of a non-intended target. Specificity can be determined based on dissociation constant through routine experiments. A domain being “specific for” a target is intended to be synonymous with a domain “directed against” said target. By “binding specifically” we also include the meaning that the domain has binding affinity specific to its target. Binding affinity or specificity in the context of a domain may be to a particular component part of a cell wall, for example, whereas binding affinity or specificity in the context of a peptide comprising at least one domain may be to a particular cell wall.

Binding affinity may be quantified by determining the dissociation constant (Kd) for a peptide or domain thereof and its target. Similarly, the specificity of binding of a peptide or domain thereof to its target may be defined in terms of the comparative dissociation constants (Kd) of the peptide or domain thereof for its target as compared to the dissociation constant with respect to the peptide or domain thereof and another, non-target molecule.

Typically, the Kd for the peptide or domain thereof with respect to the target will be at least 2-fold, preferably 5-fold, more preferably 10-fold less than Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods, and can be computed even for complex mixtures by methods such as those, for example, set forth in Caceci et al. (Byte 9:340-362, 1984). For example, the Kd may be established using a double-filter nitrocellulose filter binding assay such as that disclosed by Wong & Lohman (Proc. Natl. Acad. Sci. USA 90, 5428-5432, 1993).

A method for the evaluation of binding affinity for a peptide or domain thereof may be by ELISA. Other standard assays to evaluate the binding ability of peptides or domains thereof towards targets are known in the art, including for example, Western blots, RIAs, and flow cytometry analysis. The binding kinetics (e.g., binding affinity) of the peptide or domain thereof also can be assessed by standard assays known in the art, such as by surface plasmon resonance (e.g. Biacore™ system) analysis.

A competitive binding assay can be conducted in which the binding of the peptide or domain thereof to the target is compared to the binding of the target by another, known ligand of that target, such as an antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

A peptide of the invention or domain thereof is preferably capable of binding to its target with an affinity that is at least two-fold, 10-fold, 50-fold, 100-fold or greater than its affinity for binding to another non-target molecule.

Preferably the binding domain will bind only to its respective target, as specified above, and will not bind to any other molecule in the environment, for example in the human body. However, it will be appreciated that some degree of off-target binding may be tolerated, and the skilled person will understand how to determine whether a particular binding activity is of the required specificity or not.

A peptide of the invention is preferably capable of being expressed at a faster rate than AMP-LysA and/or AMP-LysB peptides made up of the corresponding domains. Despite the presence of additional active domains, the peptides of the invention are surprisingly not toxic to hosts such as E. coli, and so it is possible to express them in such systems without arresting host growth due to toxicity. The skilled person will realise then that the ability to express the peptides of the invention without any apparent toxicity means that the cells are able to grow faster and to a higher cell density and so overall increased peptide yield than cells expressing a toxic protein that affects the growth rate of the cell. On the other hand, AMP-LysA and/or AMP-LysB peptides are toxic to E. coli, which results in arresting cell growth and so a reduced overall peptide yield. Therefore, in some embodiments, a peptide of the invention is non-toxic to non-mycobacterial hosts, such as E. coli. The skilled person will appreciate that the toxicity of an agent can be determined by monitoring the effects of the agent on the growth of the expression host cell, and that the growth rate of a cell or culture of cells can be determined by monitoring the optical density of a culture of cells at the appropriate wavelength. For example in some embodiments, the peptide of the invention does not affect the growth rate of the expression host cell. In some embodiments the peptide of the invention reduces the growth rate of the expression host cell by less than 40%, for example by less than 35%, 30%, 25%, 20%, 15%, 10%, 5% or less than 2%. The skilled person will appreciate that the growth rate of a cell culture is typically determined during the logarithmic growth phase.

In some embodiments the peptide of the invention is less toxic to an expression host cell than the corresponding AMP-LysA and/or AMP-LysB peptides made up of the corresponding domains. The skilled person will appreciate that when comparing the toxicity of different peptides, the same amount of each peptide ought to be used. As described above, the toxicity of a peptide can be determined by monitoring the growth rate of a culture of host expression cells grown in the presence and in the absence of said peptide. In some embodiments the fusion peptide of the invention may be at least 50% less toxic, or greater, such as 60%, 70%, 80% or 90% less toxic than the corresponding AMP-LysA and/or AMP-LysB peptides made up of the corresponding domains—i.e. the fusion peptide reduces the growth rate of the expression host cell less than the corresponding AMP-LysA and/or AMP-LysB peptides. By having lower toxicity to the host in which the peptides are being expressed, the expression rate or overall yield can be increased for the peptide. As discussed above, the expression rate can be determined based on optical density (OD_{625 nm}) of the bacteria in which the peptide is being expressed, i.e. the culture density positively correlates with protein expression level. For example, a peptide of the invention, in comparison with AMP-LysA and/or AMP-LysB peptides made up of the corresponding domains, may be expressed at least 10 times (10×) faster, 9× faster, 8× faster, 7× faster, 6× faster, 5× faster, 4× faster, 3× faster or 2× faster. The rate of expression may be portrayed relative to time. For example, the rates described above may be after 1 hour, 2 hours, 3 hours, 4 hours, 5 hours, 6 hours, 7 hours, 8 hours or more of incubation in LB medium.

A peptide of the present invention preferably has a higher level of inhibitory activity against mycobacteria compared with AMP-LysA and/or AMP-LysB peptides made up of the corresponding domains. In some embodiments, a peptide of the present invention has an inhibitory activity against mycobacteria that is, compared with AMP-LysA and/or AMP-LysB peptides made up of the corresponding domains, ≥2 times (2×) higher, ≥3× higher, ≥4× higher, ≥5× higher, or more. Inhibitory activity may be assessed using known techniques, such as those used in Example 4 of the present specification.

A domain (e.g. a first domain) with activity specific to a peptidoglycan or component thereof may be an enzyme or a domain derived from an enzyme which retains the functional activity of the enzyme, e.g. an enzyme active domain (EAD), also referred to as an enzymatically active domain. Thus, in one embodiment, the first domain exerts enzyme activity on a peptidoglycan or component thereof. In one embodiment the domain is a lysin, for example an endolysin (which may be categorised as a structural endolysin, modular endolysin, and/or globular endolysin). The term “structural endolysin” may also be referred to as structural lysin, virion-associated lysin (VAL), or virion-associated peptidoglycan hydrolases (VAPGH). By “activity specific to a peptidoglycan or component thereof” we include the meaning that the domain is capable of binding or associating with a peptidoglycan or component thereof, and/or that the domain enacts a change (for example, the breakdown of the target entity) on a peptidoglycan or component thereof. For example, the domain may be capable of cleaving peptidoglycan bonds in the cell wall. The endolysin may be a peptidoglycan hydrolase enzyme, for example a LysA and/or at least one enzyme active domain (EAD) thereof. Additionally, or alternatively, the domain with activity specific to a peptidoglycan or component thereof may be a tail fibre protein with peptidoglycan hydrolysing activity, such as a structural endolysin. Additionally, or alternatively, the domain with activity specific to a peptidoglycan or component thereof may not be considered an endolysin, for example the domain may be at least one lysozyme and/or autolysin.

The targeting of the peptidoglycan layer may be achieved by at least one lysin, for example at least one endolysin, such as at least one LysA. LysA are made up of EAD that may have mechanisms of actions corresponding to amidase, endoamidases, transglycosylase (such as lytic transglycosylases), chitinase, muramidase (such as N-acetylmuramidase), glycoside hydrolase, glycosidase (glucosaminidases, e.g. N-acetyl-β-D-glucosaminidase), DD and DL carboxypeptidase, transpeptidases, epimerase, lysozyme, L-alanoyl-D-glutamate (LD), m-DAP-m-DAP (LD) D-alanyl-D-alanine carboxypeptidase and peptidase (such as N-acetylmuramoyl-L-alanine amidase, D-Alanine-meso-Diaminopimelic (DD) endopeptidase, c-D-glutamyl-meso-diaminopimelic acid (DL) peptidase, L-Alanine-D-Glutamate peptidase, cysteine protease), and γ-D-glutamyl-meso-diaminopimelic acid (DL) peptidase activity. The EAD present in a LysA may vary. For example, a LysA may comprise EAD with at least amidase, transglycosylase, chitinase, muramidase or peptidase activity, such as a LysA comprising only EAD with amidase and chitinase activity. Accordingly, in one embodiment, the targeting of the peptidoglycan layer is with LysA.

Alternatively, or additionally, the targeting of the peptidoglycan layer may be achieved using at least one EAD derived from a LysA. EAD derived from LysA may be fused with any other domain component contemplated herein. EAD derived from LysA may be fused to further LysA and/or EAD derived from LysA. For example, a first EAD derived from LysA with amidase activity may be fused to a second EAD derived from LysA with muramidase activity. Fusions may comprise 1, 2, 3, 4, 5, 6, 7, 8, 9, 10 or more EAD derived from LysA. “Derived from LysA” is to be construed as encompassing EAD derived from the same LysA or from different LysA. For example, a first EAD (with any of the above listed activities) derived from a first LysA may be fused with a second EAD (with any of the above listed activities, whether the same or different compared with the first EAD) derived from the first and/or a second LysA. Exemplary EAD include amino acid sequences corresponding to SEQ ID NOs: 326-338, as encoded by the nucleic acid sequences corresponding to SEQ ID NOs: 313-325, respectively.

The domain targeting the peptidoglycan layer may further comprise at least one cell wall binding domain (CBD). The CBD may be specific for the target cells of interest. For example, the CBD may confer specificity of a peptide to at least one mycobacteria. Alternatively, or additionally, the CBD may be classified as one that does not have specificity for Gram-negative bacteria and/or Gram-positive bacteria. For example, the CBD of a peptide described herein may be specific for mycobacteria only and not specific for Gram-negative bacteria. Multiple CBD may be present, for example as tandem repeats, and/or as multiple copies at different positions within a peptide. The presence of multiple CBD may refer to multiple copies of the same CBD and/or different CBD. For example, a peptide may comprise multiple copies of a first CBD and only one copy of a second (or further) CBD; or comprise multiple copies of a first CBD and multiple copies of a second (and/or further) CBD.

The CBD selected for use in a peptide of the present invention will depend on the intended target (or targets) of the peptide. For example, the most suitable CBD for treating M. abscessus may be a CBD derived from a mycobacteriophage that is capable of infecting M. abscessus. In some embodiments, the CBD may be capable of targeting multiple species (e.g. species of mycobacteria) based on those species sharing the targeting domain of said CBD. In some embodiments, the CBD will be determined based on the domain targeting the peptidoglycan layer. For example, if the domain targeting the peptidoglycan layer is a LysA, this may imply that the CBD is the CBD known to be associated with that specific LysA. In other cases, the CBD of a particular LysA could be swapped for (or supplemented with) a CBD from a different LysA to allow the activity of the LysA to be targeted to a different mycobacteria.

Therefore in one embodiment, the domain targeting the peptidoglycan layer is a LysA comprising a CBD. For example, in one embodiment the LysA CBD may be PGBD (Putative peptidoglycan binding domain) (pfam01471). In another embodiment, the LysA CBD may be a LGFP motif (pfam08310; superfamily cl07065).

In one embodiment, the amino acid sequence of the domain targeting the peptidoglycan layer may be selected from any one or more of SEQ ID NOs: 2, 4, 6, 8, 10, 12, 14 or 326-338; or may be encoded by any one or more of the nucleotide sequences of SEQ ID NO: 1, 3, 5, 7, 9, 11, 13 or 313-325, as follows:

Length: 1566
Type: DNA
Other information: LysA-BTCU-1
Sequence ID No: 1
ATGACAGAGCGTGTCCTGCCGTATGATCGTAATATTGTACCACAAGAAACGGGTTGGTGGTGCGGTCCGGCAGCG
ACGCAAGTTGTTCTGAATAGTCGTGGAATTATTGTACCGGAAGCGACCCTGGCAAATGAAATTGAACAAATTGAA
AATCCAGGTCGCGGTGACGATCGTGATGGAACGGATTATGTGGGGCTTATTGAACAAGTGCTTGATCGCCGTGTG
CCCGAAGCGAAATATACGAGCGTGTATATGCCTAATGATCCGCCCACCGCAGCCCAACGCGAAGCTTTATGGCAA
AATATTGTGCGCTCCATTAATGCGGGCTGGGGTGTCATTATGAATTGGGTGGCGCCGCCATCCAATAAACCACGC
GGAGTCAAAGGTAGCCCCAATCCTAAATATTCAGGTGGTACAACTTATCATTATGTGGCCTGTATGGGTTATGAT
GATACTCCGGGCGCCCGCGCCTTGTGGATTGCGGATTCTGGGTTTCAACCCTTTAATTATTGGATTTCGTTTGAT
CAAGCAGCAACGCTCATTCCACCTAAAGGGTATGCGTATGCCGCGACGACGGCTACAGCTCCTGCGCCGGCTCCA
GCCCCTGTTGATGCGGCACCCATTTTAGCACGCGCAGCTGGGATTAGCGAAGCTAAAGCACGTGAAATCTTACCC
ACCTTTCGTGATGGTTTGCGCTTAGCTGAATGTAATAATGTTCCGCGCATTGCAATGTTTATTGCACAAACAGGC
CACGAAAGCGATAATTTTAATGCCACTCAAGAATATGATCATGGCCGTAATTATGGCGATCCAAATGAAGCAACC
GATCGTTGGAAATATAAAGGGCGTACGTGGATCCAAATTACATGGCGTGGTAATTATGAGCGTTTCTCACGTTGG
TGTTTTGATCGTAAATTGGTGCCTACCCCAACATATTTTGTAGATAATCCACGCGCACTCGCGGATGTTCGTTGG
GCAGGAATTGGCGCGGCGTGGTATTGGACGGTAGAACGTCCTTCCATTAATCGCCTGTGTGATGAGCGTAATTTA
ACGGAAGTTACTCGTCTGATTAATGGTGGCACATCGTGGGAAGCCCCAACCTGGATGAAACATCGTAAAGAACGT
TATGATCGTGCGTTAGCTGTGGGTAATGATCTTTTAAAGCTGTTAGATGGCGAAGAAGAAGGGTTTCTGTCGGCG
CTTACGCCGAAAGAGCAACGTGAACTTTATGATGAAATTATGAAACGTGGGCCTAGCCGTAGCTTTCTTGCGGAT
GATGGTCGTCAAATCGAAACGCTGCTCGGTTTTATTTATAATATTGATGGAAATGCGTGGAATATTGTGAATATT
CTTGGTTGTTTGATTGGTGTACCAGAGTGTGTCGATGATATTCGTGACGTTGCCGAAAATGGAGTACGTAAAGGT
TCATATGCAGAAAGTAATCCCTGGCTCGCGCAATTTGGTCAAGAATTTTGTAAACGTTTGCTTCCCCTTGCTGGT
AAACTGTCTGAACTCTTGAATGCACAAGAATTTAAGGTTACACATAATCGCATTATTGAACAATGA
Length: 521
Molecular weight: 58.59 kDa
Type: protein
Feature: contains the following domains:
Peptidase C39 like domain; 16→152
Lysozyme like domain; 209→385
Other information: LysA-BTCU-1
Sequence ID No: 2
MTERVLPYDRNIVPQETGWWCGPAATQVVLNSRGIIVPEATLANEIEQIENPGRGDDRDGTDYVGLIEQVLDRR
VPEAKYTSVYMPNDPPTAAQREALWQNIVRSINAGWGVIMNWVAPPSNKPRGVKGSPNPKYSGGTTYHYVACMG
YDDTPGARALWIADSGFQPFNYWISFDQAATLIPPKGYAYAATTATAPAPAPAPVDAAPILARAAGISEAKARE
ILPTFRDGLRLAECNNVPRIAMFIAQTGHESDNFNATQEYDHGRNYGDPNEATDRWKYKGRTWIQITWRGNYER
FSRWCFDRKLVPTPTYFVDNPRALADVRWAGIGAAWYWTVERPSINRLCDERNLTEVTRLINGGTSWEAPTWMK
HRKERYDRALAVGNDLLKLLDGEEEGFLSALTPKEQRELYDEIMKRGPSRSFLADDGRQIETLLGFIYNIDGNA
WNIVNILGCLIGVPECVDDIRDVAENGVRKGSYAESNPWLAQFGQEFCKRLLPLAGKLSELLNAQEFKVTHNRI
IEQ
Length: 1434
Type: DNA
Other information: LysA-Bxb1
Sequence ID No: 3
ATGCCTCGCGTGGTCTATGGTCTTACACATTCTAGCAATGGCTGGCCTATGCTGAACTCGGACGAATGTGAATGG
ACCAAAATTCCGGGGACGTCAGTAACCTTACAAATTGCGAAAGGACAACCGCTGGCAATTTTGCGTGCTTTTGCG
GCAGATTTTCATGCTTATGTTGAACCTCTCCGTGATGCTGATAGTGCCTGTTGGACACCAACAAATAGTGTTCCC
AGCTCGAATCATTTGTCTGGAACTGCAATGGATCTTAATTGGAATACACATCCTTTTCAAGTTCCCGATGCGGGT
TTTGATGCTGCCAAGAAGGCTCGTGTAAAGGAACTGTTGGATTTCTATGAAGGAATGGTCTTTTGGGGTAATGAT
TGGAGCTCACCGAAAGACGCAATGCATTTTCAACTGGCTTCCTTACGTAATGGTGGCACGTTTGACACGTATGGT
AATCCCAAAACGGCTGATTTTATTGCCCGTAAAATTCGTGCCGATGGATATTCTACGTTTCGTCGTGGATCAGCG
CCTGCCTCAGCCGCGCCAATTTTGGCAGCAGCGACGGGTCTTTCGGAAGCGCGTGCCGCAGAAATTTTACCAGCA
GTGCGTAGCGGTCTGCGTGAGAGTGAATGTACTAATGTGAATCGTATTGCCATGTGGTTGGCGCAAATTGGCCAC
GAGAGCGGAAGTTTTCAATATACAGAAGAAATTGCGAAGAATGGTCGCTATGCACCATATATTGGTCGCACCTGG
ATCCAAATTACTTGGGATTATAATTATCGTTCTTTTAGCCAATGGGCCTATGCCTTTGGTATGGTCCCTACACCA
GATTATTTTGTTGTAAATTATCGTGAACTGGCGGACTTAAAATGGGCCGGAATTGGTCCGGCGTGGTATTGGACT
GTTGCGCGTCCCGATATTAATGAATTAAGTGATCGTCGTGATTTGAATACCGTAACTCGTCGCATTAATGGTGGT
ACGAATGGTCTGGCTGACCGCCAGGCGCGTTATAATCGTGCATTGGCTCAAGGTGACGCCCTCTTGCAGCTGTTG
CATGAGGAAGATGATTTTCTGTCAGCGTTAACGGATGCCGAGCAACGCGAACTGCTCGATTTAGCCCGTCAACAA
GCGAAATATAAACGTAAAAGCCGTAGCCCCTTACATTGGCCTCATGAAGGTGAAGTTGATACAATTGCGGGATTG
AGCTGGTCTACCGATGCAAATGTTCACATTCAATTAGTTGAAAAGCTGGCAGTTATTTATGGGGATCCCGTTAGC
ATTGCACTTTTATATGCCGTTTCCAATTCGGATGACCCTACCAATAATCCGGAATTAGCCAAACGTATTTTAAAA
CGTGTGAAACCGGAAGATATTACGGCAGCCCAAGTGCAAATTCAAAAGTGGCTCGCGGCGGAACAGAAATTTCAC
GCGGCGTGA
Length: 477
Molecular weight: 53.32 kDa
Type: protein
Feature: contains the following domains:
Peptidase_M15; D-alanyl-D-alanine carboxypeptidase, L-Ala-D-Glu
peptidase; 12→137
Glycoside hydrolase family 19 (GH19), Lysozyme like domain
superfamily; 190→347
Cell wall binding domain; 382→464
Other information: LysA-Bxb1
Sequence ID No: 4
MPRVVYGLTHSSNGWPMLNSDECEWTKIPGTSVTLQIAKGQPLAILRAFAADFHAYVEPLRDADSACWTPTNSVP
SSNHLSGTAMDLNWNTHPFQVPDAGFDAAKKARVKELLDFYEGMVFWGNDWSSPKDAMHFQLASLRNGGTFDTYG
NPKTADFIARKIRADGYSTFRRGSAPASAAPILAAATGLSEARAAEILPAVRSGLRESECTNVNRIAMWLAQIGH
ESGSFQYTEEIAKNGRYAPYIGRTWIQITWDYNYRSFSQWAYAFGMVPTPDYFVVNYRELADLKWAGIGPAWYWT
VARPDINELSDRRDLNTVTRRINGGTNGLADRQARYNRALAQGDALLQLLHEEDDELSALTDAEQRELLDLARQQ
AKYKRKSRSPLHWPHEGEVDTIAGLSWSTDANVHIQLVEKLAVIYGDPVSIALLYAVSNSDDPTNNPELAKRILK
RVKPEDITAAQVQIQKWLAAEQKFHAA
Length: 1482
Type: DNA
Other information: LysA-D29
Sequence ID No: 5
ATGACCTTGATTGTAACGCGCGACCATGCGCAATGGGTACACGACATGTGTCGTGCCCGTGCGGGTAATCGTTAT
GGGTATGGTGGTGCCTTTACCCTGAATCCGCGTGATACGACGGATTGTTCAGGATTAGTGTTACAAACCGCCGCG
TGGTATGGTGGCCGTAAAGATTGGATTGGCAATCGCTATGGATCAACCGAATCCTTTCGCTTAGATCATAAAATT
GTTTATGATTTAGGATTTCGCCGTCTGCCACCCGGCGGTGTCGCAGCGCTCGGCTTTACGCCTGTGATGCTGGTG
GGATTACAACATGGTGGTGGTGGTCGCTATAGCCATACTGCGTGTACCCTCATGACCATGGATATTCCGGGCGGT
CCAGTAAAAGTATCACAGCGTGGTGTTGATTGGGAAAGCCGTGGTGAGGTGAATGGGGTTGGCGTATTCTTATAT
GATGGTGCCCGTGCGTGGAATGATCCTCTGTTTCATGATTTCTGGTATTTGGATGCCAAATTGGAAGATGGTCCT
ACCCAATCCGTGGATGCAGCAGAGATTCTGGCGCGTGCCACCGGACTGGCATATAATCGCGCTGTCGCCTTACTT
CCTGCGGTCCGCGACGGGTTAATTCAAGCAGATTGTACGAATCCTAACCGTATTGCCATGTGGCTGGCTCAAATT
GGACACGAATCTGATGACTTTAAAGCAACAGCAGAATATGCGAGTGGCGACGCATATGATACTCGTACAGATTTG
GGGAATACTCCCGAAGTTGATGGTGATGGACGCCTGTATAAAGGACGCAGTTGGATTATGATTACTGGGAAAGAT
AATTATCGCGATTTTAGTCGCTGGGCGCATGGGCGCGGGCTTGTACCGACACCTGATTATTTTGTCGTGCATCCT
TTGGAACTTAGCGAATTACGTTGGGCCGGAATTGGCGCAGCTTGGTATTGGACTGTGGAACGTCCGGATATTAAT
GCGTTATCGGATCGTCGTGATCTGGAGACTGTGACTCGTCGTATTAATGGCGGTCTGACGAATCTGGACGATCGT
CGCCGTCGCTATAATTTAGCGCTGGCAGTTGGGGATCAGCTGTTAACCCTCATTGGTGACGATGATGAGCTGGCG
GACCCGACCATCCAACGTTTTATTCGTGAAATTCATGGTGCCCTCTTTAATACAGTGGTTACACAAAGCCCTTAT
GGTGATCCCCAAAATCCCGATGGTTCAGAACCGCGCTCCAATCTTTGGCAACTGCACGAATTGATTAAGAATGGT
GATGGAATGGGCCATGCGCGTTATGTGGAAGAGAGTGCACGCGCTGGCGATCTGCGTGAATTAGAACGTGTGGTG
CGTGCGGCGAAAGGTTTAGGCCGCGACCGTTCGCCGGAATTTATTGCCCGTGCGCGTAATGTACTTGCACAAATT
GAAGCCGCGAATCCGGAATATCTGCAAGCCTATATTGCGCGCAACGGCGCGTTATGA
Length: 493
Molecular weight: 54.82 kDa
Type: protein
Feature: contains the following domains:
Cystine protease; 20→156
Glycoside hydrolase family 19 (GH19); 398→483
Cell wall binding domain; 200→365
Other information: LysA-D29
Sequence ID No: 6
MTLIVTRDHAQWVHDMCRARAGNRYGYGGAFTLNPRDTTDCSGLVLQTAAWYGGRKDWIGNRYGSTESFRLDHKI
VYDLGFRRLPPGGVAALGFTPVMLVGLQHGGGGRYSHTACTLMTMDIPGGPVKVSQRGVDWESRGEVNGVGVFLY
DGARAWNDPLFHDFWYLDAKLEDGPTQSVDAAEILARATGLAYNRAVALLPAVRDGLIQADCTNPNRIAMWLAQI
GHESDDFKATAEYASGDAYDTRTDLGNTPEVDGDGRLYKGRSWIMITGKDNYRDFSRWAHGRGLVPTPDYFVVHP
LELSELRWAGIGAAWYWTVERPDINALSDRRDLETVTRRINGGLTNLDDRRRRYNLALAVGDQLLTLIGDDDELA
DPTIQRFIREIHGALFNTVVTQSPYGDPQNPDGSEPRSNLWQLHELIKNGDGMGHARYVEESARAGDLRELERVV
RAAKGLGRDRSPEFIARARNVLAQIEAANPEYLQAYIARNGAL
Length: 1461
Type: DNA
Other information: LysA-DS6A
Sequence ID No: 7
ATGATGTTGACGGTCGAGTCTTTCGCCGCTGCTATGGGTAATTCACTGTCTGTAGACCGTTATCGCCAGCTTTTC
CCAGCAGCTGTTGAGTCTATGGTTGCCTGCGGTTGTACTACCGTTAATCGCGCGGCAATGTGGCTTGCACAGGTA
GGGCACGAGTCCGGGGGTCTGCGTTGGATGGAGGAGCTGGCCAGCGGAGCCGCTTACGAGTGGCGCTCTGATTTA
GGCAACACTCAGGCAGGAGATGGGGTCCGCTTTAAAGGTCGTGGGCCTATTCAAATTACGGGACGTTACAACTAC
CGTAAGGTGAGCGAATGGGCGCATGCACAAGGCATCGTCCCTACGCCAACCTACTTTGTGGATAATCCTACTCAA
CTGGCAAGTGATCAATATGGTTTTATCGGGGTCTCGTGGTATTGGCAACATGGGGGGCCACGTCCAGGCCAAATC
AATGGCTTCGCCGACGCAGGAGATATTTTATCCGGTTCGCGCTGTGTAAATGGGTGGGTAACGACCCCAAATGGC
ATGCCGGACCGCACCGAGCGTTGGAACCGTTGTCGTGCAATGGGCGACCAAATCCTTCCAGCAGGCGCAGGTACT
GATAATGGAGGGGATGGTATGGCTTGGACTGGAGATCCGGTGTGGTTAGAAGACGTGTTGCGCCCCGTATTAGGG
GACCGTCTTCGCGTCCTTCCTTCGTGGCAAATGTACGGTCACGGTGATTTCAAAGATATCCGTGGAGTTATGGTG
CATCATACTGGGAACGCGCGTGAAACAGCAGAGTCCATTCGTAAGGGCCGTCCAGATTTGCGTGGCCCGTTGTCG
AACATCCACATCGCGCCCGATGGAACCGTTACACTTGTTGCCGCAGGTGTTTGCTGGCACGCCGGTGCCGGGTCA
TACCCGTGGTTACCTACCAATAATGCCAATTGGCATATGATCGGAATTGAATGCGCATGGCCTACTATTCGTCCG
AACGGGACATACGACGAGCGCGAGCCTTGGCCCGATGCGCAAATTATCGCTATGCGCGATACGTGTGCCGCCTTA
ACCAAACGTCTGGGCTGGGACGCCTCGCGCGTTATTGGCCACAAGGAATATGCCGGGGCTTCTCAGGGAAAATGG
GATCCAGGGAATTTGGATATGGGTTGGTTCCGTGGTGAGGTTGCTAAAGCGATGCGCGGCGAGTTCGATCCAAAG
GCGCCAGCACCAGCACCCAACCCACCGGCAGACAATGACAATGGGGGCTTCCTTATGGCGCTTAACGATAAGCAA
CAACATGATTTATACAATGCGGTTATGACTATCGCAGCAGTCGCTGCCGACATCCAAACACAATTACGCGGTCCC
GGCCTTCGTGGTTGGCCCCAATTGGGCCGCAATGCGAACGGTGAGGCATTAACCCTGGTGGATGCTGTGGGAGTC
ATCAAGGCCATTGCGGATCGCGAGGCCGGGCGTTGA
Length: 485
Molecular weight: 52.71 kDa
Type: protein
Feature: contains the following domains:
Lysozyme like domain superfamily; 20→190
N-acetylmuramoyl-L-alanine amidase (Am_2); 233→380
Peptidoglycan binding domain; 225→396
Other information: LysA-DS6A
Sequence ID No: 8
MLTVESFAAAMGNSLSVDRYRQLFPAAVESMVACGCTTVNRAAMWLAQVGHESGGLRWMEELASGAAYEWRSDLG
NTQAGDGVRFKGRGPIQITGRYNYRKVSEWAHAQGIVPTPTYFVDNPTQLASDQYGFIGVSWYWQHGGPRPGQIN
GFADAGDILSGSRCVNGWVTTPNGMPDRTERWNRCRAMGDQILPAGAGTDNGGDGMAWTGDPVWLEDVLRPVLGD
RLRVLPSWQMYGHGDFKDIRGVMVHHTGNARETAESIRKGRPDLRGPLSNIHIAPDGTVTLVAAGVCWHAGAGSY
PWLPTNNANWHMIGIECAWPTIRPNGTYDEREPWPDAQIIAMRDTCAALTKRLGWDASRVIGHKEYAGASQGKWD
PGNLDMGWFRGEVAKAMRGEFDPKAPAPAPNPPADNDNGGFLMALNDKQQHDLYNAVMTIAAVAADIQTQLRGPG
LRGWPQLGRNANGEALTLVDAVGVIKAIADREAGR
Length: 1209
Type: DNA
Other information: LysA-Giles
Sequence ID No: 9
ATGCCTGTCTATGCGATTCAGTCTGGAACTGTTATTTACGCCGGTGCCGCATCGGGATATGGTGGTCCTGACCCT
GCAGGCTGGCTTGTTATTGATTCTGATGATGCCGAGGGTGGCGGCTGTTTAGAGTATGGTCATATTGTCCGTGAG
GTAAATCGTGGTGATCATGTGACGGCGGGTCAACGTATTGGACATATTAATCCAAATAATCGTACGAATGGTGGA
GTGGCCCCGCATCTTCATGTAACTGACTGGCCACATGCGTATGGGCAAGGCAGCCGTCAAAATGTGATGGCACGC
TTGAGTGGAGCCCGCGAGCCAGAAGCCCAAGCGGCCCCAACCCCCACAGAGCAAGGCACTGTTGTTGGTGACCCG
GTTTGGTTGGCGGATGTAGTGCGCCCAACTGTAGCTAAATTTGCGGAGTATCCTGGCTGGCGTAATCGTGGTCAC
GGTGATTTTAAGGATATCCGTGGAGTTATGGTTCATCATACAGGTGGGCCCGCCTCTGCAGCCTCAATTGCGAAT
GGCCGCCCAGACCTTGCCGGCCCTCTTGCACAGCTGCATATTTCGCGTGATGGGACAGTTACTGTCGTAGCTGTA
GGAGTGGCTTGGCATGCGGGTGCAGGCAGCTATCCATGGTTACCGACCAACATGGGTAATTGGCACTTGATTGGT
ATTGAATGTGAGTGGCCATATGGTGAGCCGGGAATTAATGAGCGTAATGCCTATACAGTACGCTGGCGTGATCCA
GAGATTATTGCGCTGCGTAATACATGTGCGGCAATTTTACTGCGCCTTGGCCTCGGGGTAGATCGCCTGATTGGC
CATAAGGATTATGCCGGCCGTGCTCAAGGCAAGTGGGACCCGGGAAATATGAGCATGGACTGGCTGCGTGGCGAG
GTGCGTAAAGATATGGATGGCTTTGTATTTCCCGGTGAGGATATTGTAAATGTGACCCCGCCACCAGTCCCGGCC
CCTGTCCCTGCCCCGCCAGCGACTGCGAATGTTCTTCTCCATCGTGGTATGAGTGGACCTAATGTTGTCAAATTA
CAGACAACTCTCCGTCGTTGGTATTCAAAACTGGCGGTAGACGGTGATTTTGGCCCCCATACAGAGGCATGTGTA
CGCGATCGCCAACGCATTTATGGGTTGGACGTGGATGGTATTGTCGGACCATTAACTGCGGCCAAAATCGGCCTG
GTTTTGTGA
Length: 402
Molecular weight: 43 kDa
Type: protein
Feature: contains the following domains:
Peptidase family M23; 2→93
Amidase_2; N-acetyl-β-D-muramidase; 123→298
Cell wall binding domain; 320→350
Peptidoglycan binding domain; 351→398
Other information: LysA-Giles
Sequence ID No: 10
MPVYAIQSGTVIYAGAASGYGGPDPAGWLVIDSDDAEGGGCLEYGHIVREVNRGDHVTAGQRIGHINPNNRTNGG
VAPHLHVTDWPHAYGQGSRQNVMARLSGAREPEAQAAPTPTEQGTVVGDPVWLADVVRPTVAKFAEYPGWRNRGH
GDFKDIRGVMVHHTGGPASAASIANGRPDLAGPLAQLHISRDGTVTVVAVGVAWHAGAGSYPWLPTNMGNWHLIG
IECEWPYGEPGINERNAYTVRWRDPEIIALRNTCAAILLRLGLGVDRLIGHKDYAGRAQGKWDPGNMSMDWLRGE
VRKDMDGFVFPGEDIVNVTPPPVPAPVPAPPATANVLLHRGMSGPNVVKLQTTLRRWYSKLAVDGDFGPHTEACV
RDRQRIYGLDVDGIVGPLTAAKIGLVL
Length: 1155
Type: DNA
Other information: LysA-Ms6
Sequence ID No: 11
ATGACTACAAAGGACCAGGTAGCGCAGATTACAATTGCAGAGGCAAAAGCCCGTGGGTATACTCGTTCAGAGTGT
TTAGCCATTATGTCTACGTTTTATCAGGAAAGCGGTTGGAATGATACTATTTGGGATCCTACGCATACAACGTAT
GGAATCGCGCAACAAGATGGTAGTTATCCCCATCGTTTTGATGGCGCGGCCGCGCAGATTAAGGGTTTCTTTGAT
AAACTGGATGTTTGGCGTGCAAAGCCGGGCGCATCGACAGACATTTGGCTTAATATTTGTTGGATGCAACAAGCG
CCGAATTGGCCGTCGGCCGATTATTGGTATGCGAATGGTCGTCGTGCTTATCTGACAGAGATTAAAAGTCGTATT
GCGACGGTTACGCCGTATCTGGATAAATATTGGCCTGCTGACGGCGGCACGGCGGTTCCAGATGAGCCTCGTCCG
GACTTTAATGAGTTCCCAATTTGGTCAAATAATAATTCGGCGCGTTCAGGTAAACCGACTATGTTTCTGATTCAT
ACGCAGGAAGGCGGTGGTGGTGATGCCGCAGCTGAAAATTTGGCCAAATGGTTTCAAAATGGTAATGGAGTGTCC
TATCATTATACCATTAGCCAGGCAAGCGACGGCGGCGTAACCGTAGTGGACTGTGTAGATACGGATCGTGCGGCG
TGGAGCGTCGGAAATGCTAATAGTATTAGTATTAATTTGTGTTTTGCTGGATCACGCGCGAGCTGGATGCGCGAC
CAATGGATGAAACAATCGAATGCTATTGATGTTGCCGCGTATCTGGCTGTTCAAGATGCCAAGAAATATGGTTTT
ACGCCCTTGGTCGTACCGCCGCCGTACACCAACGGCCGTCCGGGGATTAGCGATCATCGCTGGGTTACTGATGTA
TTTAAATGGGGTACCCATACGGATGTTGGTGATTGGTTTCCTTGGGATTATTTTGCGGAGCGCGTTAATCATTGG
GCAAATGGCGGTAAAACGGAACCGGAGCCACCTAAAGTTAAGCGTTTTCCTGATGATTGGAGCGATCGTGAGATT
CTGGTTGAAATTCTTCGCCAGTTACGTGGGTATAATTTGACCGGGTGGCCACAATTGGGTGGCAAGACACTGGTA
GATGCTGTGGCGGAGTTGCTGGGACATTGA
Length: 384
Molecular weight: 43 kDa
Type: protein
Feature: contains the following domains:
Hydrolase; 5→154
Amidase_2; N-acetyl-β-D-muramidase; 175→299
Cell wall binding domain; 313→376
Other information: LysA-Ms6
Sequence ID No: 12
MTTKDQVAQITIAEAKARGYTRSECLAIMSTFYQESGWNDTIWDPTHTTYGIAQQDGSYPHRFDGAAAQIKGFFD
KLDVWRAKPGASTDIWLNICWMQQAPNWPSADYWYANGRRAYLTEIKSRIATVTPYLDKYWPADGGTAVPDEPRP
DENEFPIWSNNNSARSGKPTMFLIHTQEGGGGDAAAENLAKWFQNGNGVSYHYTISQASDGGVTVVDCVDTDRAA
WSVGNANSISINLCFAGSRASWMRDQWMKQSNAIDVAAYLAVQDAKKYGFTPLVVPPPYTNGRPGISDHRWVTDV
FKWGTHTDVGDWFPWDYFAERVNHWANGGKTEPEPPKVKRFPDDWSDREILVEILRQLRGYNLTGWPQLGGKTLV
DAVAELLGH
Length: 1644
Type: DNA
Other information: LysA-TM4
Sequence ID No: 13
ATGTCATTTACGCGCTTTCTCCAAGACGATCCCTTGCTGACTCGTGAACAGGTAATGGCAGAACTCATCCGCGTT
GCTGATGAATTGAATATGCCTGATAAACGTGGAGCATGTGTGATCGCAGGAATGACTATCTCACAGGAAGTTGGT
GTGAAAGATAATGATCCCCCATTTGAACGCCGTTTCTGGTGTCCAGCAAATCGTGCGGATCCAGAGTCTTTTAAT
TATCCACATGATAGTGAGTCCAATGATGGACGTTCAGTGGGGTATTTTCAACAACAAAAGGGCCCAAATGGTGAA
CTTTGGTGGGGCACTACCGCGAGTGAAATGAATTTACATAGTGCAGCCACCCAATTCATGACTCGTTTAAAAGCC
GCGGGTTATAATGCCTCTAATGCGCAAGCCGCAAATGATTCTGCTCAAGCTATTCAACGTTCAGGTGTTCCACAA
GCCTATAAACAGTGGTGGGATGATATCAATCGTTTATATGATAAAGTAAAAGGTAGCGGCGGCGGACCCGCACCG
GCTCCCAAACCACCTCAAAGCGGTCCTTGGACTGGTGATCCTGTTTGGTTAGCAGATGTATTACGTGCAGAAGGC
TTGAATGTTGTTGAACTCCCTGGTTGGTTAGATCGTGGTCATGGAGATATGGGTCGTCTGTGGGGCGTCGTATGT
CACCATACTGGTAGTGACAATACTCCAAGCTCTGAAATCGCATTCCATCCCAGCCTGGGTCTTTGTTCCCAAATC
CATCTTGCCCGTAATGGTACGGTTACACTTTGTGGCGTTGGTATTGCGTGGCACGCAGGAGTGGGATCGTACCCG
GGGTTGCCGGAAGATAATGCAAATGCCGTGACAATTGGTATTGAAGCACAAAATTCGGGTACATACGATGGTGCT
CCTCATCGCACCAACTGGCCGGATGCACAGTATGATGCGTACGTCAAATGTTGTGCGGCTATTTGTCGTCGTCTG
GGAGTCCGTGCAGACCATGTAATTTCGCATAAAGAGTGGGCGGGCCGTAAACAGGGTAAGTGGGACCCGGGAGCG
ATTGATATGAATATTTTCCGTGCGGATGTTCAACGTCGTATTGATGCGCACCAGCCGAATGGTGAAGATGACTTT
ATGGCGGCTCTGAGCGCGGATGAACAACGTGAAGTTTTGAATCTTTTGCGTGTTTTAGCGGATCGCCGCTTTGTG
AGTCGTTCACCTTTTCGTCATCTCGGTGAAGGTCCATCCGAGACGGTAGCGGGCTTTGGTTTAAATACGGATGGA
CTGAACCATGCTCAATATACCATCGAACTCGCACGTCTCGGGGATCCTACCCATTTAGCGTTACTCCGTGAAGTA
GCTAGTGCGGAAGGGGATTCTCGTTACCCGGATCGCCAATATGATGCGAAATTGGCGAAACGTGTCTTGGCTGAG
ATTGAAGGTGCGGCGACCGCGCCTGCGAAACCTTCTACTCCTTCAGCGCCAACGGAACCAGCACCGGAAGCACCT
ACTCCACCGGTGAAAGCGGCTTGTGCACTCAGCGCCGCGGGCTGTGTAGTAGCGGGGAGCACTTCAGGTGGTGGT
TGTGCGTTATCGACAGATGGTACGGGGAAATGTGTGGTCACTGCGGCCACTGATGGTGGCGCGGCATGA
Length: 547
Molecular weight: 58.58 kDa
Feature: contains the following domains:
M23B Peptidase; 67→165
Amidase_2; N-acetyl-β-D-muramidase; 187→357
Cell wall binding domain; 401→484
Other information: LysA-TM4
Sequence ID No: 14
MSFTRFLQDDPLLTREQVMAELIRVADELNMPDKRGACVIAGMTISQEVGVKDNDPPFERRFWCPANRADPESEN
YPHDSESNDGRSVGYFQQQKGPNGELWWGTTASEMNLHSAATQFMTRLKAAGYNASNAQAANDSAQAIQRSGVPQ
AYKQWWDDINRLYDKVKGSGGGPAPAPKPPQSGPWTGDPVWLADVLRAEGLNVVELPGWLDRGHGDMGRLWGVVC
HHTGSDNTPSSEIAFHPSLGLCSQIHLARNGTVTLCGVGIAWHAGVGSYPGLPEDNANAVTIGIEAQNSGTYDGA
PHRTNWPDAQYDAYVKCCAAICRRLGVRADHVISHKEWAGRKQGKWDPGAIDMNIFRADVQRRIDAHQPNGEDDF
MAALSADEQREVLNLLRVLADRRFVSRSPFRHLGEGPSETVAGFGLNTDGLNHAQYTIELARLGDPTHLALLREV
ASAEGDSRYPDRQYDAKLAKRVLAEIEGAATAPAKPSTPSAPTEPAPEAPTPPVKAACALSAAGCVVAGSTSGGG
CALSTDGTGKCVVTAATDGGAA
Length: 460
Type: DNA
Sequence ID: EADA1 (SEQ ID NO: 313)
TATTGTACCACAAGAAACGGGTTGGTGGTGCGGTCCGGCAGCGACGCAAGTTGTTCTGAATAGTCGTGGAATTAT
TGTACCGGAAGCGACCCTGGCAAATGAAATTGAACAAATTGAAAATCCAGGTCGCGGTGACGATCGTGATGGAAC
GGATTATGTGGGGCTTATTGAACAAGTGCTTGATCGCCGTGTGCCCGAAGCGAAATATACGAGCGTGTATATGCC
TAATGATCCGCCCACCGCAGCCCAACGCGAAGCTTTATGGCAAAATATTGTGCGCTCCATTAATGCGGGCTGGGG
TGTCATTATGAATTGGGTGGCGCCGCCATCCAATAAACCACGCGGAGTCAAAGGTAGCCCCAATCCTAAATATTC
AGGTGGTACAACTTATCATTATGTGGCCTGTATGGGTTATGATGATACTCCGGGCGCCCGCGCCTTGTGGATTGC
GGATTCTGGG
Length: 153
Molecular weight: 16.7 kDa
Type: protein
Feature: Peptidase_C39_2
Sequence ID No: EADA1 (SEQ ID NO: 326)
IVPQETGWWCGPAATQVVLNSRGIIVPEATLANEIEQIENPGRGDDRDGTDYVGLIEQVLDRRVPEAKYTSVYMP
NDPPTAAQREALWQNIVRSINAGWGVIMNWVAPPSNKPRGVKGSPNPKYSGGTTYHYVACMGYDDTPGARALWIA
DSG
Length: 537
Type: DNA
Sequence ID: EADA2 (SEQ ID NO: 314)
GAAATCTTACCCACCTTTCGTGATGGTTTGCGCTTAGCTGAATGTAATAATGTTCCGCGCATTGCAATGTTTATT
GCACAAACAGGCCACGAAAGCGATAATTTTAATGCCACTCAAGAATATGATCATGGCCGTAATTATGGCGATCCA
AATGAAGCAACCGATCGTTGGAAATATAAAGGGCGTACGTGGATCCAAATTACATGGCGTGGTAATTATGAGCGT
TTCTCACGTTGGTGTTTTGATCGTAAATTGGTGCCTACCCCAACATATTTTGTAGATAATCCACGCGCACTCGCG
GATGTTCGTTGGGCAGGAATTGGCGCGGCGTGGTATTGGACGGTAGAACGTCCTTCCATTAATCGCCTGTGTGAT
GAGCGTAATTTAACGGAAGTTACTCGTCTGATTAATGGTGGCACATCGTGGGAAGCCCCAACCTGGATGAAACAT
CGTAAAGAACGTTATGATCGTGCGTTAGCTGTGGGTAATGATCTTTTAAAGCTGTTAGATGGCGAAGAAGAAGGG
TTTCTGTCGGCG
Length: 178
Molecular weight: 21 kDa
Type: protein
Feature: Lysozyme like domain
Sequence ID No: EADA2 (SEQ ID NO: 327)
EILPTFRDGLRLAECNNVPRIAMFIAQTGHESDNFNATQEYDHGRNYGDPNEATDRWKYKGRTWIQITWRGNYER
FSRWCFDRKLVPTPTYFVDNPRALADVRWAGIGAAWYWTVERPSINRLCDERNLTEVTRLINGGTSWEAPTWMKH
RKERYDRALAVGNDLLKLLDGEEEGELSA
Length: 189
Type: DNA
Sequence ID: EADA3 (SEQ ID NO: 315)
AGCTCGAATCATTTGTCTGGAACTGCAATGGATCTTAATTGGAATACACATCCTTTTCAAGTTCCCGATGCGGGT
TTTGATGCTGCCAAGAAGGCTCGTGTAAAGGAACTGTTGGATTTCTATGAAGGAATGGTCTTTTGGGGTAATGAT
TGGAGCTCACCGAAAGACGCAATGCATTTTCAACTGGCT
Length: 63
Molecular weight: 7.12 kDa
Type: protein
Feature: Peptidase_M15_4
Sequence ID No: EADA3 (SEQ ID NO: 328)
SSNHLSGTAMDLNWNTHPFQVPDAGFDAAKKARVKELLDFYEGMVFWGNDWSSPKDAMHFQLA
Length: 423
Type: DNA
Sequence ID: EADA4 (SEQ ID NO: 316)
AGCGGTCTGCGTGAGAGTGAATGTACTAATGTGAATCGTATTGCCATGTGGTTGGCGCAAATTGGCCACGAGAGC
GGAAGTTTTCAATATACAGAAGAAATTGCGAAGAATGGTCGCTATGCACCATATATTGGTCGCACCTGGATCCAA
ATTACTTGGGATTATAATTATCGTTCTTTTAGCCAATGGGCCTATGCCTTTGGTATGGTCCCTACACCAGATTAT
TTTGTTGTAAATTATCGTGAACTGGCGGACTTAAAATGGGCCGGAATTGGTCCGGCGTGGTATTGGACTGTTGCG
CGTCCCGATATTAATGAATTAAGTGATCGTCGTGATTTGAATACCGTAACTCGTCGCATTAATGGTGGTACGAAT
GGTCTGGCTGACCGCCAGGCGCGTTATAATCGTGCATTGGCTCAAGGT
Length: 141
Molecular weight: 16.3 kDa
Type: protein
Feature: Lysozyme like domain
Sequence ID No: EADA4 (SEQ ID NO: 329)
SGLRESECTNVNRIAMWLAQIGHESGSFQYTEEIAKNGRYAPYIGRTWIQITWDYNYRSFSQWAYAFGMVPTPDY
FVVNYRELADLKWAGIGPAWYWTVARPDINELSDRRDLNTVTRRINGGTNGLADRQARYNRALAQG
Length: 507
Type: DNA
Sequence ID: EADA5 (SEQ ID NO: 317)
GCCGCGCCAATTTTGGCAGCAGCGACGGGTCTTTCGGAAGCGCGTGCCGCAGAAATTTTACCAGCAGTGCGTAGC
GGTCTGCGTGAGAGTGAATGTACTAATGTGAATCGTATTGCCATGTGGTTGGCGCAAATTGGCCACGAGAGCGGA
AGTTTTCAATATACAGAAGAAATTGCGAAGAATGGTCGCTATGCACCATATATTGGTCGCACCTGGATCCAAATT
ACTTGGGATTATAATTATCGTTCTTTTAGCCAATGGGCCTATGCCTTTGGTATGGTCCCTACACCAGATTATTTT
GTTGTAAATTATCGTGAACTGGCGGACTTAAAATGGGCCGGAATTGGTCCGGCGTGGTATTGGACTGTTGCGCGT
CCCGATATTAATGAATTAAGTGATCGTCGTGATTTGAATACCGTAACTCGTCGCATTAATGGTGGTACGAATGGT
CTGGCTGACCGCCAGGCGCGTTATAATCGTGCATTGGCTCAAGGTGACGCCCTCTTG
Length: 168
Molecular weight: 19 kDa
Type: protein
Feature: Chitinase, GH19 family
Sequence ID No: EADA5 (SEQ ID NO: 330)
AAPILAAATGLSEARAAEILPAVRSGLRESECTNVNRIAMWLAQIGHESGSFQYTEEIAKNGRYAPYIGRTWIQI
TWDYNYRSFSQWAYAFGMVPTPDYFVVNYRELADLKWAGIGPAWYWTVARPDINELSDRRDLNTVTRRINGGTNG
LADRQARYNRALAQGDAL
Length: 525
Type: DNA
Sequence ID: EADA6 (SEQ ID NO: 318)
ACCTTGATTGTAACGCGCGACCATGCGCAATGGGTACACGACATGTGTCGTGCCCGTGCGGGTAATCGTTATGGG
TATGGTGGTGCCTTTACCCTGAATCCGCGTGATACGACGGATTGTTCAGGATTAGTGTTACAAACCGCCGCGTGG
TATGGTGGCCGTAAAGATTGGATTGGCAATCGCTATGGATCAACCGAATCCTTTCGCTTAGATCATAAAATTGTT
TATGATTTAGGATTTCGCCGTCTGCCACCCGGCGGTGTCGCAGCGCTCGGCTTTACGCCTGTGATGCTGGTGGGA
TTACAACATGGTGGTGGTGGTCGCTATAGCCATACTGCGTGTACCCTCATGACCATGGATATTCCGGGCGGTCCA
GTAAAAGTATCACAGCGTGGTGTTGATTGGGAAAGCCGTGGTGAGGTGAATGGGGTTGGCGTATTCTTATATGAT
GGTGCCCGTGCGTGGAATGATCCTCTGTTTCATGATTTCTGGTATTTGGATGCCAAATTGGAAGATGGTCCTACC
Length: 175
Molecular weight: 19.32 kDa
Type: protein
Feature: Cysteine protease
Sequence ID No: EADA6 (SEQ ID NO: 331)
TLIVTRDHAQWVHDMCRARAGNRYGYGGAFTLNPRDTTDCSGLVLQTAAWYGGRKDWIGNRYGSTESFRLDHKIV
YDLGFRRLPPGGVAALGFTPVMLVGLQHGGGGRYSHTACTLMTMDIPGGPVKVSQRGVDWESRGEVNGVGVFLYD
GARAWNDPLFHDFWYLDAKLEDGPT
Length: 555
Type: DNA
Sequence ID: EADA7 (SEQ ID NO: 319)
CAATCCGTGGATGCAGCAGAGATTCTGGCGCGTGCCACCGGACTGGCATATAATCGCGCTGTCGCCTTACTTCCT
GCGGTCCGCGACGGGTTAATTCAAGCAGATTGTACGAATCCTAACCGTATTGCCATGTGGCTGGCTCAAATTGGA
CACGAATCTGATGACTTTAAAGCAACAGCAGAATATGCGAGTGGCGACGCATATGATACTCGTACAGATTTGGGG
AATACTCCCGAAGTTGATGGTGATGGACGCCTGTATAAAGGACGCAGTTGGATTATGATTACTGGGAAAGATAAT
TATCGCGATTTTAGTCGCTGGGCGCATGGGCGCGGGCTTGTACCGACACCTGATTATTTTGTCGTGCATCCTTTG
GAACTTAGCGAATTACGTTGGGCCGGAATTGGCGCAGCTTGGTATTGGACTGTGGAACGTCCGGATATTAATGCG
TTATCGGATCGTCGTGATCTGGAGACTGTGACTCGTCGTATTAATGGCGGTCTGACGAATCTGGACGATCGTCGC
CGTCGCTATAATTTAGCGCTGGCAGTTGGG
Length: 185
Molecular weight: 20.7 kDa
Type: protein
Feature: Glyco_hydro_19
Sequence ID No: EADA7 (SEQ ID NO: 332)
QSVDAAEILARATGLAYNRAVALLPAVRDGLIQADCTNPNRIAMWLAQIGHESDDFKATAEYASGDAYDTRTDLG
NTPEVDGDGRLYKGRSWIMITGKDNYRDFSRWAHGRGLVPTPDYFVVHPLELSELRWAGIGAAWYWTVERPDINA
LSDRRDLETVTRRINGGLTNLDDRRRRYNLALAVG
Length: 582
Type: DNA
Sequence ID: EADA8 (SEQ ID NO: 320)
TTAACTGTAGAGAGCTTCGCTGCTGCTATGGGTAACAGCTTGTCAGTTGACCGTTACCGCCAACTGTTTCCAGCG
GCGGTTGAGTCAATGGTGGCTTGTGGATGCACCACCGTGAATCGCGCGGCGATGTGGCTGGCACAGGTCGGCCAT
GAGTCTGGGGGTTTGCGCTGGATGGAGGAGTTAGCGTCAGGAGCCGCATATGAGTGGCGCTCGGACCTTGGAAAC
ACGCAGGCGGGAGACGGCGTACGCTTTAAAGGCCGCGGACCCATCCAAATCACGGGGCGTTATAATTATCGTAAA
GTGAGTGAGTGGGCTCATGCGCAGGGCATTGTGCCTACGCCGACATACTTCGTGGATAACCCAACTCAATTGGCA
TCGGATCAATACGGCTTCATCGGTGTTTCATGGTACTGGCAGCACGGCGGGCCACGTCCAGGCCAAATTAACGGC
TTCGCAGACGCAGGGGACATCCTGTCGGGATCGCGCTGCGTGAACGGATGGGTGACAACGCCAAACGGGATGCCC
GACCGTACTGAACGTTGGAATCGTTGTCGCGCTATGGGAGATCAAATCCTTCCGGCA
Length: 194
Molecular weight: 21.51 kDa
Type: protein
Feature: Glyco hydro 19
Sequence ID No: EADA8 (SEQ ID NO: 333)
LTVESFAAAMGNSLSVDRYRQLFPAAVESMVACGCTTVNRAAMWLAQVGHESGGLRWMEELASGAAYEWRSDLGN
TQAGDGVRFKGRGPIQITGRYNYRKVSEWAHAQGIVPTPTYFVDNPTQLASDQYGFIGVSWYWQHGGPRPGQING
FADAGDILSGSRCVNGWVTTPNGMPDRTERWNRCRAMGDQILPA
Length: 525
Type: DNA
Sequence ID: EADA9 (SEQ ID NO: 321)
GATCGTCTTCGTGTGCTTCCCTCCTGGCAGATGTACGGTCATGGCGATTTTAAAGATATCCGCGGCGTTATGGTC
CACCATACTGGTAACGCTCGCGAAACTGCCGAGTCTATCCGTAAAGGACGCCCTGATTTACGTGGTCCGCTGTCC
AATATTCACATCGCACCGGATGGCACGGTGACATTAGTCGCCGCCGGAGTCTGTTGGCATGCAGGAGCCGGGTCG
TACCCATGGTTACCAACTAATAACGCCAACTGGCACATGATCGGTATTGAATGCGCTTGGCCTACAATCCGTCCA
AACGGAACGTATGATGAACGCGAGCCATGGCCTGATGCTCAAATCATCGCTATGCGTGACACCTGCGCTGCACTT
ACCAAACGTCTGGGATGGGACGCATCTCGCGTGATCGGTCATAAAGAGTACGCTGGCGCTTCGCAAGGTAAATGG
GACCCCGGCAACTTGGATATGGGATGGTTTCGTGGAGAGGTAGCTAAGGCAATGCGTGGTGAATTTGACCCTAAA
Length: 175
Molecular weight: 19.5 kDa
Type: protein
Feature: Amid2B
Sequence ID No: EADA9 (SEQ ID NO: 334)
DRLRVLPSWQMYGHGDFKDIRGVMVHHTGNARETAESIRKGRPDLRGPLSNIHIAPDGTVTLVAAGVCWHAGAGS
YPWLPTNNANWHMIGIECAWPTIRPNGTYDEREPWPDAQIIAMRDTCAALTKRLGWDASRVIGHKEYAGASQGKW
DPGNLDMGWFRGEVAKAMRGEFDPK
Length: 279
Type: DNA
Sequence ID: EADA10 (SEQ ID NO: 322)
CCTGTCTATGCGATTCAGTCTGGAACTGTTATTTACGCCGGTGCCGCATCGGGATATGGTGGTCCTGACCCTGCA
GGCTGGCTTGTTATTGATTCTGATGATGCCGAGGGTGGCGGCTGTTTAGAGTATGGTCATATTGTCCGTGAGGTA
AATCGTGGTGATCATGTGACGGCGGGTCAACGTATTGGACATATTAATCCAAATAATCGTACGAATGGTGGAGTG
GCCCCGCATCTTCATGTAACTGACTGGCCACATGCGTATGGGCAAGGCAGCCGT
Length: 93
Molecular weight: 9.72 kDa
Type: protein
Feature: Peptidase M23
Sequence ID No: EADA10 (SEQ ID NO: 335)
PVYAIQSGTVIYAGAASGYGGPDPAGWLVIDSDDAEGGGCLEYGHIVREVNRGDHVTAGQRIGHINPNNRTNGGV
APHLHVTDWPHAYGQGSR
Length: 447
Type: DNA
Sequence ID: EADA11 (SEQ ID NO: 323)
TATCCTGGCTGGCGTAATCGTGGTCACGGTGATTTTAAGGATATCCGTGGAGTTATGGTTCATCATACAGGTGGG
CCCGCCTCTGCAGCCTCAATTGCGAATGGCCGCCCAGACCTTGCCGGCCCTCTTGCACAGCTGCATATTTCGCGT
GATGGGACAGTTACTGTCGTAGCTGTAGGAGTGGCTTGGCATGCGGGTGCAGGCAGCTATCCATGGTTACCGACC
AACATGGGTAATTGGCACTTGATTGGTATTGAATGTGAGTGGCCATATGGTGAGCCGGGAATTAATGAGCGTAAT
GCCTATACAGTACGCTGGCGTGATCCAGAGATTATTGCGCTGCGTAATACATGTGCGGCAATTTTACTGCGCCTT
GGCCTCGGGGTAGATCGCCTGATTGGCCATAAGGATTATGCCGGCCGTGCTCAAGGCAAGTGGGACCCGGGA
Length: 149
Molecular weight: 16.18 kDa
Type: protein
Feature: Amidase 2
Sequence ID No: EADA11 (SEQ ID NO: 336)
YPGWRNRGHGDFKDIRGVMVHHTGGPASAASIANGRPDLAGPLAQLHISRDGTVTVVAVGVAWHAGAGSYPWLPT
NMGNWHLIGIECEWPYGEPGINERNAYTVRWRDPEIIALRNTCAAILLRLGLGVDRLIGHKDYAGRAQGKWDPG
Length: 297
Type: DNA
Sequence ID: EADA12 (SEQ ID NO: 324)
GCAAATCGTGCGGATCCAGAGTCTTTTAATTATCCACATGATAGTGAGTCCAATGATGGACGTTCAGTGGGGTAT
TTTCAACAACAAAAGGGCCCAAATGGTGAACTTTGGTGGGGCACTACCGCGAGTGAAATGAATTTACATAGTGCA
GCCACCCAATTCATGACTCGTTTAAAAGCCGCGGGTTATAATGCCTCTAATGCGCAAGCCGCAAATGATTCTGCT
CAAGCTATTCAACGTTCAGGTGTTCCACAAGCCTATAAACAGTGGTGGGATGATATCAATCGTTTATATGAT
Length: 99
Molecular weight: 11.07 kDa
Type: protein
Feature: M23B peptidase
Sequence ID No: EADA12 (SEQ ID NO: 337)
ANRADPESFNYPHDSESNDGRSVGYFQQQKGPNGELWWGTTASEMNLHSAATQFMTRLKAAGYNASNAQAANDSA
QAIQRSGVPQAYKQWWDDINRLYD
Length: 384
Type: DNA
Sequence ID: EADA13 (SEQ ID NO: 325)
TGGGGCGTCGTATGTCACCATACTGGTAGTGACAATACTCCAAGCTCTGAAATCGCATTCCATCCCAGCCTGGGT
CTTTGTTCCCAAATCCATCTTGCCCGTAATGGTACGGTTACACTTTGTGGCGTTGGTATTGCGTGGCACGCAGGA
GTGGGATCGTACCCGGGGTTGCCGGAAGATAATGCAAATGCCGTGACAATTGGTATTGAAGCACAAAATTCGGGT
ACATACGATGGTGCTCCTCATCGCACCAACTGGCCGGATGCACAGTATGATGCGTACGTCAAATGTTGTGCGGCT
ATTTGTCGTCGTCTGGGAGTCCGTGCAGACCATGTAATTTCGCATAAAGAGTGGGCGGGCCGTAAACAGGGTAAG
TGGGACCCG
Length: 128
Molecular weight: 13.72 kDa
Type: protein
Feature: M23B peptidase
Sequence ID No: EADA13 (SEQ ID NO: 338)
WGVVCHHTGSDNTPSSEIAFHPSLGLCSQIHLARNGTVTLCGVGIAWHAGVGSYPGLPEDNANAVTIGIEAQNSG
TYDGAPHRTNWPDAQYDAYVKCCAAICRRLGVRADHVISHKEWAGRKQGKWDP

A domain (e.g. a second domain) with activity specific to an ester linkage may be specific to the ester linkage between mycolic acid and arabinogalactan. By “activity specific to an ester linkage” we include the meaning that the domain is capable of binding or associating with an ester linkage, and/or that the domain enacts a change (for example, the breakdown of the target entity) on an ester linkage. For example, the domain may be capable of cleaving ester linkages/ester bonds in the cell wall. For example, the second domain may target an ester link between any one of more of the following components: trehalose, mycolates, arabinogalactan, peptidoglycan, muramic acid; optionally wherein the components are in the following pairs: arabinogalactan and mycolates; trehalose and mycolates; arabinogalactan and peptidoglycan linkages; and/or arabinogalactan and muramic acid.

The domain with activity specific to an ester linkage may be an enzyme or a domain derived from an enzyme which retains the functional activity of the enzyme, e.g. an enzyme active domain (EAD). Thus, in one embodiment, the second domain exerts enzyme activity on an ester linkage. In one embodiment the domain is an enzyme with lipolytic activity.

In one embodiment the domain is selected from any member of the alpha/beta hydrolase family, or a domain derived from a member of the alpha/beta hydrolase family. Such a domain may be a mycolyl arabinogalactan esterase, for example a LysB and/or at least one EAD thereof. For example, such a domain may be a mycolyl-arabinogalactan-peptidoglycan (mAGP) hydrolase. Such a domain may act specifically on particular components of the aforementioned targets. For example, the domain may target the ester linkage of mycolic acids. In a further example, the domain may have one or more of the following enzyme activities: alpha/beta hydrolase, esterase, lipase, cutinase, trehalose dimycolate hydrolase (TDMH), Pectinesterase, CheB methylesterase, Glycerophosphoryl diester phosphodiesterase, Plant invertase/pectin methylesterase inhibitor, Carboxylesterase family, Calcineurin-like phosphoesterase, Putative esterase, Thioesterase domain, Hemagglutinin esterase, Calcineurin-like phosphoesterase superfamily domain, Pectinacetylesterase, Putative serine esterase, Esterase PHB depolymerase, Esterase-like activity of phytase, Chitin recognition protein, Glycosyl hydrolase all families, Amidase, Lipase all families, GDSL-like Lipase/Acylhydrolase, Partial alpha/beta-hydrolase lipase region, GDSL-like Lipase/Acylhydrolase family, Secretory lipase, Patatin-like phospholipase, Carboxylesterase, Variant-surface-glycoprotein phospholipases all families, Putative lysophospholipase, Alpha/beta-hydrolase superfamily, Hydrolase, haloacid dehalogenase-like hydrolase, epoxide hydrolase and dehalogenases, peroxidase, or combinations thereof.

In one embodiment the domain is a mycolylarabinogalactan esterase, such as LysB. Thus, in one embodiment the domain is LysB and/or at least one enzyme active domain (EAD) thereof.

The targeting of the ester linkage (also termed “ester bond”, both of which are interchangeable herein) refers to an ester link or bond between two entities. For example, the ester link may be between a mycolic acid (MA) that is bonded (i.e. via an ester bond) to the terminal pentaarabinofuranosyl units of arabinogalactan (AG). Accordingly, in some embodiments, the domain targeting (or having specificity/affinity for) an ester link may be a domain that targets and/or disrupts the binding of MA to the terminal pentaarabinofuranosyl units of AG. This may be achieved by at least one Lysin B, for example at least one LysB. LysB are made up of enzyme active domains (EAD) that may have mechanisms of actions corresponding to (i.e. specific activity resembling): alpha/beta hydrolase activity, esterase activity, lipase activity, protease activity, TDMH, cutinase activity, trehalose dimycolate hydrolase (TDMH), Pectinesterase, CheB methylesterase, Glycerophosphoryl diester phosphodiesterase, Plant invertase/pectin methylesterase inhibitor, Carboxylesterase family, Calcineurin-like phosphoesterase, Putative esterase, Thioesterase domain, Hemagglutinin esterase, Calcineurin-like phosphoesterase superfamily domain, Pectinacetylesterase, Putative serine esterase, Esterase PHB depolymerase, Esterase-like activity of phytase, Chitin recognition protein, Glycosyl hydrolase all families, Amidase, Lipase all families, GDSL-like Lipase/Acylhydrolase, Partial alpha/beta-hydrolase lipase region, GDSL-like Lipase/Acylhydrolase family, Secretory lipase, Patatin-like phospholipase, Carboxylesterase, Variant-surface-glycoprotein phospholipases all families, Putative lysophospholipase, Alpha/beta-hydrolase superfamily, Hydrolase, haloacid dehalogenase-like hydrolase, epoxide hydrolase and dehalogenases, peroxidase, or any combinations thereof. Accordingly, in one embodiment, the targeting of the ester linkage is with LysB.

Each of the above activities may arise from a single EAD that is derived from a LysB. However, each LysB may comprise different EADs. For example, a LysB may comprise EADs with at least alpha/beta hydrolase activity, esterase activity, lipase activity, protease activity, TDMH, and/or cutinase activity; or a LysB may comprise only EAD with alpha/beta hydrolase activity, esterase activity, lipase activity, protease activity, TDMH, and/or cutinase.

Alternatively, or additionally, the targeting of the ester linkage may be achieved using at least one EAD derived from a LysB. EAD derived from LysB may be fused with any other domain component contemplated herein. EAD derived from LysB may be fused to further LysB and/or EAD derived from LysB. For example, a first EAD derived from LysB with lipase activity (and/or any previously specified activity) may be fused to a second EAD derived from LysB with cutinase activity (and/or any previously specified activity). Fusions may comprise 1, 2, 3, 4, 5, 6, 7, 8, 9, 10 or more EADs derived from LysB. “Derived from LysB” is to be construed as encompassing EADs derived from the same LysB or from different LysB. For example, a first EAD (with any of the above listed activities) derived from a first LysB may be fused with a second EAD (with any of the above listed activities, whether the same or different compared with the first EAD) derived from the first and/or a second LysB.

The domain with activity specific to an ester linkage may further comprise at least one cell wall binding domain (CBD). The CBD may be specific for the target cells of interest. For example, the CBD may confer specificity of a peptide to at least one mycobacteria. Alternatively, or additionally, the CBD may be classified as one that does not have specificity for Gram-negative bacteria and/or Gram-positive bacteria. For example, the CBD of a peptide described herein may be specific for mycobacteria only and not specific for Gram-negative bacteria. Multiple CBD may be present, for example as tandem repeats, and/or as multiple copies at different positions within a peptide. The presence of multiple CBD may refer to multiple copies of the same CBD and/or different CBD. For example, a peptide may comprise multiple copies of a first CBD and only one copy of a second (or further) CBD; or comprise multiple copies of a first CBD and multiple copies of a second (and/or further) CBD. In one embodiment, the CBD of this domain may be LysB Saal.

The wildtype forms of lytic enzymes of interest for the present invention (e.g. LysA and LysB) comprise more than one catalytic domain (i.e. EAD) representing the different classes of peptidoglycan hydrolases and esterases (or in general alpha/beta hydrolases). Accordingly, LysA and/or LysB may refer to enzymes that have a single type of activity (whether comprised of one domain with this activity or multiple domains with the same activity) or with multiple types of activity (as discussed in Payne et al., 2012, PLOS One, 7 (3), e34052). Types of activity may also be referred to as mechanisms of action. Although wildtype LysA is composed of 1-3 EAD and 1-3 cell binding domains (CBD), the activity required of the first domain can be achieved with one or more of EAD and/or CBD.

The LysA (and/or EAD derived therefrom) and LysB (and/or EAD derived therefrom) may derive from the same organism or different organisms. For example, at least one LysA may be derived from the same source (e.g. the same mycobacteriophage) as the LysB (or EAD derived from either). Alternatively, at least one LysA (or EAD therefrom) may be derived from a different source (e.g. different mycobacteriophages, phages, bacteria, human, animal, and/or plant sources) as the LysB (or EAD therefrom). Where multiple LysA and/or LysB are present in a fusion peptide, some may derive from the same organism, while others derive from different organisms. For example, in a fusion peptide comprising 2 LysA and 2 LysB, a first LysA may be derived from the same organism as a first LysB, while a second LysA is derived from a different organism to a second LysB (either of which may derive from the same or different organism as the first LysA and LysB).

An EAD derived from a lysin would be considered “a portion thereof” with respect to that lysin. For example, an EAD (or multiple EAD) from a specific LysA would be considered a portion thereof said LysA. Thus, use of a LysA and/or LysB throughout is intended to also mean LysA (and/or LysB) or a portion thereof.

In one embodiment, the amino acid sequence of the domain with activity specific to an ester linkage may be selected from any one or more of the amino acid sequences of SEQ ID NOs: 16, 18, 20, 22, 24, 26, 28, 30 or 32, or may be encoded by any one or more of the nucleotide sequences of SEQ ID NOs: 15, 17, 19, 21, 23, 25, 27, 29 or 31, as follows:

Length: 756
Type: DNA
Other information: LysB-D29
Sequence ID No: 15
ATGAGCAAGCCCTGGCTGTTCACCGTTCACGGCACGGGCCAGCCCGATCCCCTCGGGCCTGGCCTGCCTGCCGAT
ACGGCACGCGACGTACTTGACATCTACCGGTGGCAGCCCATCGGCAACTACCCCGCTGCGGCCTTCCCGATGTGG
CCGTCGGTCGAGAAGGGTGTCGCCGAGCTGATCCTGCAGATCGAGCTGAAGCTGGACGCGGACCCCTACGCGGAC
TTCGCGATGGCGGGTTACTCGCAGGGAGCCATCGTGGTTGGCCAGGTGCTCAAGCACCACATCCTGCCTCCGACG
GGCAGGCTCCACAGGTTCCTGCACCGGCTCAAGAAGGTCATCTTCTGGGGTAATCCCATGCGGCAGAAGGGCTTT
GCCCACTCTGACGAGTGGATCCACCCGGTCGCTGCCCCTGACACCCTCGGAATCCTCGAGGACCGGCTCGAAAAC
CTGGAGCAGTACGGCTTCGAGGTCCGCGACTACGCCCACGACGGTGACATGTACGCCTCCATCAAAGAGGACGAC
CTGCACGAATACGAGGTCGCCATCGGCCGGATCGTGATGAAGGCCAGCGGCTTCATCGGTGGCCGGGACTCCGTG
GTAGCCCAGCTCATCGAGCTTGGCCAGCGTCCGATCACCGAGGGAATTGCGTTGGCGGGAGCCATCATCGACGCC
CTCACGTTCTTCGCCCGCTCTCGTATGGGCGACAAGTGGCCGCACCTCTACAACCGCTACCCGGCGGTCGAGTTC
CTACGACAGATCTGA
Length: 254
Molecular weight: 28.5 kDa
Type: protein
Feature: contains the following domains:
Cutinase domain; 41→69
LysinB C-terminal linker domain; 176→244
PE-PPE; 51→125
Other information: LysB-D29
Sequence ID No: 16
MSKPWLFTVHGTGQPDPLGPGLPADTARDVLDIYRWQPIGNYPAAAFPMWPSVEKGVAELILQIELKLDADPYAD
FAMAGYSQGAIVVGQVLKHHILPPTGRLHRFLHRLKKVIFWGNPMRQKGFAHSDEWIHPVAAPDTLGILEDRLEN
LEQYGFEVRDYAHDGDMYASIKEDDLHEYEVAIGRIVMKASGFIGGRDSVVAQLIELGQRPITEGIALAGAIIDA
LTFFARSRMGDKWPHLYNRYPAVEFLRQI
Length: 846
Type: DNA
Other information: LysB-Omega
Sequence ID No: 17
ATGTTGAACGGTGAATTTTACGTTCCGGCAGGGACAATCATTACTGCCAACGGGACAGGGGTTCCAGATGGTGCG
GGGCCCGGATTTGGTGCCGATCTGGCTCGCGAACTGGAGCGTGTGGCTGAAGGCATGTGGATTTGGAAATGGATC
AACTACCCAGCAGCAGTAATGCCGATGCGTCCGAGCATTAATACGTTGCGTTTGGCTTTAAAAGAGATGATCCGT
ACCACTCCGGGCAAAATTGTTTTATCGGCGTACTCTCAATCGGCCGTGGCTTTTGCTTACGTATGGCGCGACGAC
ATTCTTAACCCCGATGGTGAACTTCACGACCGCCTGGATGATATTGTAGCAGTAATCTTGTACGGCAACCCAGTT
CGCGCCCCTGGGATTGCTTATGGCAACGAGTTAGGCGGTCAATCTGCCCCCGGCAAGCTGAATGGGCATGTCACC
GGCGGCATCGCCGGGCCTGATTGTTTGCGCCCGGAAGAGTGCGTCCATCCCGTCACTGGGCGTCGCATTGTACTG
GACTTCGCTAATGCCGGTGATTIGTACGCTGCTGCACCCGTTGGCGCGGAACCCIGGGTCAAAGAGACTGAGGTA
GGCCATAACGAGACGCTTATTTACGAAGCTGTTATGGATTTTAATGGCCGCGATATCCTTGCATTCGCCAAAGAG
ATCGCACAGATTCTTACAATGCCCTTGAGCCAGGTCGTACCGCTGGTACAAGCAATTATCAACGGGTTAAGCTTT
CTTGTCCAGGGCCCGAAAGCTCCGCACTGGACGTACACGATTGGCCCGGCAGTCGATTACTTAGTCCGCACGGGC
AATGAACTTCGTAAAAATTGA
Length: 281
Molecular weight: 30.3 kDa
Type: protein
Feature: contains Cutinase domain; 21→139
Other information: LysB-Omega
Sequence ID No: 18
MLNGEFYVPAGTIITANGTGVPDGAGPGFGADLARELERVAEGMWIWKWINYPAAVMPMRPSINTLRLALKEMIR
TTPGKIVLSAYSQSAVAFAYVWRDDILNPDGELHDRLDDIVAVILYGNPVRAPGIAYGNELGGQSAPGKLNGHVT
GGIAGPDCLRPEECVHPVTGRRIVLDFANAGDLYAAAPVGAEPWVKETEVGHNETLIYEAVMDENGRDILAFAKE
IAQILTMPLSQVVPLVQAIINGLSFLVQGPKAPHWTYTIGPAVDYLVRTGNELRKN
Length: 1002
Type: DNA
Other information: LysB-Saal
Sequence ID No: 19
ATGCGCATCGACGGTCAATATGTGGGACTGGGGCTTGGGGATTCTTCAGACGAGATCCGTAAAATCAAGGCGTTC
ATGCGCCGCAAATTCGCGAGCTATGCCGGAGATTTAGCGGATACTCCTTTGTACGATGAAGCTATGACCGCAGCA
GTGGCCGAGATGCAAGCGCGCTACAGTGCGGCTGGTCAACTICGTGCTGGATTGTACATTCCCGGTATCGTAGGA
GCCGAAACTAAGTATGTGATGGGTTACTTACCCCGCCCCGTCGTTGATACTCGTCCGGTACTGATCACTGTGTGT
GGTACGGGTGTTCCATGGTGGATTGGGCCTGATGCAGATACTGCTCGCGCTGTGGAGGATAAGTACCTGTGGCAA
CCGATCGGTTATCCTGCAGCCCCTTTTCCAATGGGAAAATCCATCGCGGCGGCTATTACCGAGACTCACAACCAA
GCCAATCGCTGGCGCGAGCGCATCGAAACTCACGGCGCGGCCTTGGCTGGCTATAGCCAGGGTGCAGTGGTAGTG
TCAGAGCTTTGGATGAACCACATTGCGCCGGAGGATGGTTCCCTTCATTGGATGAAACCACACATTGAAAAAGCC
GTCACTTGGGGGAACCCAAATCGCGAGTTGGGGCACGTATGGGGGGACCACGGGGGTTCTCCTCGTGCGCCGTCT
AATACCCAAGGCGTTTCAAGTAACGGAATGCGTGATACGCCCCCATGGTGGCGTGATTATGCTCACCAGGGIGAC
CITTATGCGTGTACAGAGCCAGGGGATACTCAAGAGGTGCGTAACGCGATTTGGCAAATTGTACGCGACTTGGAT
TTATTTACAGGGCCCGACAGTTTGTTAGCCCAGGTTGTAGAGTTGGTGCAGGCCCCTCTGCCCGAAGCTATCGCT
ATCACAAAGGCGCTTTTGGATGCCGGTATGTTCTTCGCTAAACGTACAGGCCCACATGTGGACTATAACGTACAG
CCAGCGATCGATTACCTGCGCACATGA
Length: 308
Molecular weight: 33.8 kDa
Type: protein
Feature: contains the following domains:
Putative peptidoglycan binding domain; 38→58
PE-PPE; 136→224
Cutinase domain; 120→208
Other information: LysB-Saal
Sequence ID No: 20
MRRKFASYAGDLADTPLYDEAMTAAVAEMQARYSAAGQLRAGLYIPGIVGAETKYVMGYLPRPVVDTRPVLITVC
GTGVPWWIGPDADTARAVEDKYLWQPIGYPAAPFPMGKSIAAAITETHNQANRWRERIETHGAALAGYSQGAVVV
SELWMNHIAPEDGSLHWMKPHIEKAVTWGNPNRELGHVWADHGGSPRAPSNTQGVSSNGMRDTPPWWRDYAHQGD
LYACTEPGDTQEVRNAIWQIVRDLDLFTGPDSLLAQVVELVQAPLPEAIAITKALLDAGMFFAKRTGPHVDYNVQ
PAIDYLRT
Length: 1038
Type: DNA
Other information: LysB-Ds6A
Sequence ID No: 21
ATGACCTGGATTGGCTGGCAGCAAGGTATGGGGGGAGAGCCGGTAGCCGCTGCGAAGCGTGAGCTTCGTCGCAAA
TTCAGTTATGCTAAACATCTGGATGACAGCGACGTATTTGATCTGGAGTTATTGGCCGTTTTGATCGAGTACCAA
CAAAAAAAGAACGCGACTGGTTATACACCTCGCCTGCGCGAAGACGGCGTTCTTGATTGGGCCACTCAAGTTGCG
ATTGGTACGGTGCAAACTGGGCCCCCTCCTAAGGCAGGCACGTTATTTACTGTGCAAGGAACTGGCGTTGATATG
TGGACAGGACCCCCGGCGGACACGGCTCGTGCCGTGGAGGGCGACTGGGAATGGCAGCCGATTGGCAATTACCCT
GCTTCACCGTTCCCAATGTGGGCTTCCATCTGGCAGGGGATTGAAGAACTGCGCTTTCAGATTCGTCGCCATGCT
ACGGAAAAACCGGGGAAAGCCATTGCCCTGGCAGGTTTCTCACAGGGTGCCGTAGTCGTAAGTTGGGTCTACAAG
TGGGACATTGCACTGCCCGGTGGCATGCTGCACGACTTGCTGCCACAGGTGGCAGCGGGTGTAACGTGGGGTAAT
CCCATGGCAGAAAAAGGCAAGTTTCATGGTAACCGCTTCGCCGGCTGGCCTGTGCCTGCCGGAATGGGGATCAAC
CGTGACCGCTTGGAAAATACCCCCGCATTATGGCTTGACTTTGGGCACGGTCGCAATTCACCTTGGGGCCAAGAT
ATTTATTGCGACCGCCCGGATAATTTGGCTGGTCAGGACATGGAGCTTATTTGTGACCTGGTCATGGCTCAGGAC
CTGTGGCGCTTTGTGACAGACTTCGCAGTAAAAGCTGGGAACGTGATGGCGTCGCCTGAGACGGAGTTACCCGCA
ATCTTGGATGCAGTTACACAAGCTGGTATGTTTTTCATTGTTAATCGTACTGCCCCGCATCTGACTTATGACGTA
GGTCCTGCCATCGATTACCTTCGCCAAGTTGCGCGCGACCGCCGCTTGGCTTTAGCCGCGTGA
Length: 345
Molecular weight: 38.17 kDa
Type: protein
Feature: contains the following domains:
LysinB C-terminal linker domain; 260→325
PE-PPE; 132→219
Cutinase domain; 119→206
Other information: LysB-Ds6A
Sequence ID No: 22
MTWIGWQQGMAGEPVAAAKRELRRKFSYAKHLDDSDVEDLELLAVLIEYQQKKNATGYTPRLREDGVLDWATQVA
IGTVQTGPPPKAGTLFTVQGTGVDMWTGPPADTARAVEGDWEWQPIGNYPASPFPMWASIWQGIEELRFQIRRHA
TEKPGKAIALAGFSQGAVVVSWVYKWDIALPGGMLHDLLPQVAAGVTWGNPMAEKGKFHGNRFAGWPVPAGMGIN
RDRLENTPALWLDFGHGRNSPWGQDIYCDRPDNLAGQDMELICDLVMAQDLWRFVTDFAVKAGNVMASPETELPA
ILDAVTQAGMFFIVNRTAPHLTYDVGPAIDYLRQVARDRRLALAA
Length: 981
Type: DNA
Other information: LysB-Obama12
Sequence ID No: 23
ATGCTGAAGTTAGGATCAAACGGGCTGATGGTTGCGGCGTGGACGGCAGTTATGCGTATCCGTTTCGCTTCGTAT
GCTCTTGGTGTAAACGGTTTACCGATCAAGGTAGATGGCTACTTTGGTTATGATGAAGAAAAAGTGCAAAAGGAG
TATCAACGTCGTACAGGACAGTTCCCGTCTGGACAAGTATCGCGCGAGGATCTGCATCGTCTGGGTTTATTGCCA
ACTTTAATCAGCATTCACGGCACAGGTCAAGCAGACCCTTTTGGCATCGGGTACCCGGCAGACATTGCTCGCCGC
GTGCTTGATCTTTATTGGTGGCAGCCGTTAGGCAACTACCCGGCGAAAGCTGTCCCTATGAACGGATCGGTTGAC
CAGGGTGAACGTGAGGGCGTCCGTTTGATCTCTGACCCCCTTATCGTGCCAGGTCCAACAGCGTTTGTAGATTAT
TCCCAAGGTTCAGTGATTGGAGGGCGCTTACGCAACCGCATGCGCCGTAAGGAGCTGCGCGGAGAGCTTGTCGCC
GCAGCCAGTTTTGGAAATCCAATGCGTCTGCGCGGCCATTACGCAGGCAACGTAGACCCTGGCGGTGAAGGTATT
GATCCACGTCAAGAGCTGGCTGCTGAACCATTCCGCATCGAGTTAGCTGCGAAAGGCGACTTGTACACGACTTGC
CCTGGCGGTCAGTCAGGCGAAATGGAACGCGCCATTTATCATGCGGTTTTTAGCAAGTTCATCGGAGAAGATACT
CTTATCGAGCAAGTATGGGAACTTGCTAAAAATCCATTTCGTGAAGTCCCGGCCGCCGTGAAGGCTATTGTGCGC
GGCGGAATGTTCGCGATCCGTGGCACAGGTCCTCATGTTCGCTACCATATTGATCAATGCCCAGGGACGGGAATG
ACATATTACGAATACGCGGTCAAGCATTTGCGCGACACCGCAGATGCGCGTTTGCGCCGCATTGTCGCGTCTGTA
ACATGA
Length: 317
Molecular weight: 35 kDa
Type: protein
Feature: contains LysinB C-terminal linker domain; 221→383
Other information: LysB-Obama12
Sequence ID No: 24
MVAAWTAVMRIRFASYALGVNGLPIKVDGYFGYDEEKVQKEYQRRTGQFPSGQVSREDLHRLGLLPTLISIHGTG
QADPFGIGYPADIARRVLDLYWWQPLGNYPAKAVPMNGSVDQGEREGVRLISDPLIVPGPTAFVDYSQGSVIGGR
LRNRMRRKELRGELVAAASFGNPMRLRGHYAGNVDPGGEGIDPRQELAAEPFRIELAAKGDLYTTCPGGQSGEME
RAIYHAVFSKFIGEDTLIEQVWELAKNPFREVPAAVKAIVRGGMFAIRGTGPHVRYHIDQCPGTGMTYYEYAVKH
LRDTADARLRRIVASVT
Length: 759
Type: DNA
Other information: LysB-Enkosi
Sequence ID No: 25
ATGTCTAAGCCTGTGCTGTTGACTGCTAGTGGGACGGGTGCGGATATGTGGTCTGGATATCCGGCGGATCTTGCG
CGTCGCGTCGAGGACCTTTACTACTTTCAGCCAATCTACTATGGTCCAGGTGGGATTCCGGCGACGGTACCAATG
GGGGCGAGCGCACAGTCTGGCGTAGATATGGGAGTACGTCTGGTACTGGAGTGCGAGGCTCGTCCGTCGCGCGAC
GTCCCTGATGGGTATGCCTTGTGTGGCTACTCTCAAGGGGGCATGGTTGTATCCGCGTTGTTGGACGAGTTTCGC
ACGGGCCGCCTTCGCCACTTGAATGGCAAATTGATGGCGGGCAGCACCTTTGGAAACCCGTGGCGCGAGCTTGAT
TCTAACGGGGGGCGCGGCATCAGCGACCGTCGCATTGTGGGGACCCCCGACTTCTGGGTGGATGAGTTCGACCCA
GGAGACATTTATGCAAACGTGCCTAACAATGATGTCGGGGATGATATGACTGCCATTTTTAAGCTGGTACAGCTG
CGCTCACTGGCTGACCTGATCGGTATTGGATCGATTATGGATCGCGTAATGGACGTTCTTCGCGGCCCATTGAGC
GGGATGCGCTCCGTAGTTGAGGCGATTGTTCGTGGCTTATTATTTTTCGGCCGTAAGCCGCCAACAGCGCCCCAT
ACAGAGTACCATTTGCGCGAGGTCCGTCCGGGAGTGACGTATTTTGAACATGCAGTCGCCCACATGCGCGCTATG
GCCGCCTGA
Length: 237
Molecular weight: 26 kDa
Type: protein
Feature: contains the following domains :
LysinB C-terminal linker domain; 147→218
Alpha/Beta hydrolase family 1→224
Other information: LysB-Enkosi
Sequence ID No: 26
MWSGYPADLARRVEDLYYFQPIYYGPGGIPATVPMGASAQSGVDMGVRLVLECEARPSRDVPDGYALCGYSQGGM
VVSALLDEFRTGRLRHLNGKLMAGSTEGNPWRELDSNGGRGISDRRIVGTPDFWVDEFDPGDIYANVPNNDVGDD
MTAIFKLVQIRSLADLIGIGSIMDRVMDVLRGPLSGMRSVVEAIVRGLLFFGRKPPTAPHTEYHLREVRPGVTYF
EHAVAHMRAMAA
Length: 966
Type: DNA
Other information: LysB-Bxz2
Sequence ID No: 27
ATGCCGTTACGCGTGGGCAGCAATGATGCAAATACCGGTGGTCTTGTATCACGCTGGCAAAAGACCATGTTAGCG
CGTTATGCCGCGTATGCGAAAGCTTATGATGGCGGCCCATTACGTGTGGATGGGTACTTTGGTTATGATGATGCC
GATGTGCAACGTGAATATGAGCGTCGTACACATCAAGTTGTGGATGGGGAAGTTAGCGATGCGGATCTGCGTGCC
TTGGGGTTGGAAGCCGCAAAACGTTGGCTTTTCACCGTGCATGGAACTGGCCAAGCAGATCCTCTTGGGCCAGGC
TTGCCTGCAGATACCGCCCGTGCCGTCCTGGATAAATATACGTGGCAACCGATTGGAAATTATCCTGCACGTGCA
TTTCCAATGTGGTCGTCCATTATGGATGGCGCGCGCGAACTCCGTTCGCAAATTGCTTCCAAAAGTGGCGAAGTT
AATCTCGCAGGTTATAGTCAGGGTGCAGTAGTGGTTGGGCAAGTTCTGAAACATGATATTATGGATCCTAAAGGT
TCTCTCCATCATCGCTTAGGTGACGTGCGTAAAGTTGTACTTTGGGGTAACCCGATGCGTCAACGTGGCATTGCC
CATTTTGACGAATGGATCCATCCTGTCGCTGGACCCGATTCTTATGGTATTTTGGACGATCGCCTGGAAGGATTG
GAGAAAGCGCCTTTTGAAATTCGTGATTATGCCCATGCCGGCGATATGTATGCGAGCATTACCGATGGTGATAAA
GATGAATATAAAATTGCCATTTGTAAAATTGTGATGACCGCTACCGATTTCTATCGCGGTCCAAATAGCGTGGTC
AGCCAGCTCATTGAATTGGGTCAACGCCCTCTGACTGAAGGTATTGCGATGGCGCTTGCAATTATTGATACCCTT
CGCTTCTTTACCAATACGGCTCATGGGTATAATATTGGTCCGGCGATTGATTTTCTTCGCTCATGA
Length: 321
Molecular weight: 35.41 kDa
Type: protein
Feature: contains the following domains:
PE-PPE; 130→199
Cutinase 149→200
LysinB C-terminal linker domain; 221→283
Other information: LysB-Bxz2
Sequence ID No: 28
MPLRVGSNDANTGGLVSRWQKTMLARYAAYAKAYDGGPLRVDGYFGYDDADVQREYERRTHQVVDGEVSDADLRA
LGLEAAKRWLFTVHGTGQADPLGPGLPADTARAVLDKYTWQPIGNYPARAFPMWSSIMDGARELRSQIASKSGEV
NLAGYSQGAVVVGQVLKHDIMDPKGSLHHRLGDVRKVVLWGNPMRQRGIAHFDEWIHPVAGPDSYGILDDRLEGL
EKAPFEIRDYAHAGDMYASITDGDKDEYKIAICKIVMTATDFYRGPNSVVSQLIELGQRPLTEGIAMALAIIDTL
RFFTNTAHGYNIGPAIDFLRS
Length: 765
Type: DNA
Other information: LysB-L5
Sequence ID No: 29
ATGAGTAAACCGTGGTTATTTACTGTGCATGGAACCGGACAACCCGATCCTCTTGGTCCCGGGCTCCCGGCAGAC
ACAGCTCGTGATGTTTTGGATATTTATCGTTGGCAACCAATTGGTAATTATCCAGCCGCCGCATTTCCCATGTGG
CCCAGCGTGGAGAAAGGGGTTGCCGAATTAATTCTCCAAATTGAACTTAAATTGGATGCGGACCCATATGCCGAT
TTTGCCTTGGCGGGTTATTCTCAAGGTGCGATTGTTGTAGGACAAGTTCTGAAACATCATATTATTAATCCTCGC
GGACGTTTGCATCGTTTTCTTCATCGTCTGCGCAAAGTAATTTTCTGGGGCAACCCCATGCGTCAGAAAGGTTTC
GCGCATACTGATGAATGGATCCATCAAGTGGCCGCAAGTGATACTATGGGTATTCTGGAAGATCGTTTGGAAAAT
CTTGAACAGTATGGTTTCGAAGTTCGTGATTATGCGCATGATGGTGATATGTACGCGAGTATTAAAGAAGATGAT
ATGCATGAATATGAAGTTGCGATCGGTCGTATTGTTATGTCTGCGCGTCGCTTTATTGGCGGAAAAGATTCAGTA
ATTGCGCAACTGATTGAACTGGGCCAACGCCCAATTTGGGAAGGCATTGCAATGGCTCGCGCTATTATTGATGCG
CTTACCTTCTTCGCGAAATCTACACAGGGGCCTTCGTGGCCTCACCTTTATAATCGTTTTCCAGCCGTAGAATTT
CTGCGTCGCATTTGA
Length: 254
Molecular weight: 28.84 kDa
Type: protein
Feature: contains the following domains:
Alpha/Beta hydrolase family 176→244
LysinB C-terminal linker domain; 3→251
Other information: LysB-L5
Sequence ID No: 28
MSKPWLFTVHGTGQPDPLGPGLPADTARDVLDIYRWQPIGNYPAAAFPMWPSVEKGVAELILQIELKLDADPYAD
FALAGYSQGAIVVGQVLKHHIINPRGRLHRFLHRLRKVIFWGNPMRQKGFAHTDEWIHQVAASDTMGILEDRLEN
LEQYGFEVRDYAHDGDMYASIKEDDMHEYEVAIGRIVMSARRFIGGKDSVIAQLIELGQRPIWEGIAMARAIIDA
LTFFAKSTQGPSWPHLYNRFPAVEFLRRI
Sequence ID No: 31
Length: 999
Type: DNA
Other information: LysB-Ms6
Sequence ID No: 30
ATGCGTATCGACGGCCAATATGTTGGATTAGGACCAGGGGATCGTTCGGACGAGATCCGCAAGATCAAAGCGTTC
ATGCGTCGCAAGTTCTCCTACGCAGCGACGCTGGCCGACACTGAGTTCTACGACGAGGCCATGACCGCTGTAGTC
GCCGAGATGCAATCCCGTTACAACACTGCTGGTCAGCTCCGCGACGGGTTGTACATCCCCGGGATTATCAACGCT
GAAACCAAGTACGTTATGGGATATTTGTCACGTCCAGTAATCGACACACGTCCAGTCCTGTTTACCGTATGCGGA
ACCGGCGTGCCTTGGTGGGTCGGCCCAGACGCCGACACCGCTCGCGCTGTTGAGGACCAATATTTATGGCAACCG
ATTGGTTATCCGGCCGCGCCCTTCCCAATGGGTCGTTCCATCACCGCGGGGATCACTGAGGCACACAACCAGGCG
AACCGCTGGCGCGAACGTATCGAAACTCACGGCACCGCCCTCGCAGGTTATTCTCAAGGCGCAGTCGTTCTTTCT
GAGTTATGGATGAACCACATCGCACCGGAAGACGGCTCGCTTCGCTGGATGAAACCACATGTACGTAAAGCAGTG
ACGTGGGGCAACCCGAACCGCGAATTAGGCCACGTGTGGGCCGATCACGGCGGCTCTCCTATGGCGCCAAGTAAC
ACTCAGGGCGTGTCGTCAAACGGAATGCGCAACACGCCTGACTGGTGGCGCGACTACGCCCACCAAGGCGACCTG
TACGCTTGCACCGAACCGGGAGACACCCAAGAGGTTCGCAACGCGATCTGGCAGATCGTCCGCGACCTGGACCTG
TTCACCGGACCAGATTCACTCCTTGCCCAAGTGATCGAACTGGCCCAAGCCCCTCTCCCTGAGACGATCGCGATC
ACTCGCGCGATCCTGGACGCGGGTATGTTCTTCGCGAAACGCACAGGCCCACACGTAGACTACAACCCGCAGCCA
GCGATCGACTATCTCCGCACGTGA
Length: 307
Molecular weight: 34.21 kDa
Type: protein
Feature: contains the following domains:
Putative peptidoglycan binding domain; 15→58
PE-PPE; 134→224
Cutinase; 120→208
LysinB C-terminal linker domain; 234→298
Other information: LysB-Ms6
Sequence ID No: 32
MRRKFSYAATLADTEFYDEAMTAVVAEMQSRYNTAGQLRDGLYIPGIINAETKYVMGYLSRPVIDTRPVLFTVCG
TGVPWWVGPDADTARAVEDQYLWQPIGYPAAPFPMGRSITAGITEAHNQANRWRERIETHGTALAGYSQGAVVLS
ELWMNHIAPEDGSLRWMKPHVRKAVTWGNPNRELGHVWADHGGSPMAPSNTQGVSSNGMRNTPDWWRDYAHQGDL
YACTEPGDTQEVRNAIWQIVRDLDLFTGPDSLLAQVIELAQAPLPETIAITRAILDAGMFFAKRTGPHVDYNPQP
AIDYLRT

In some embodiments, the domain with activity specific to an ester linkage is selected from the group consisting of D29 (SEQ ID NO: 6), Omega (SEQ ID NO: 18), Saal (SEQ ID NO: 20), Obama12 (SEQ ID NO: 24), Bxz2 (SEQ ID NO: 28), L5 (SEQ ID NO: 30), MS6 (SEQ ID NO: 32) and TDMH (SEQ ID NO: 34).

A domain (e.g. a third domain) with membrane permeabilising, destabilising and/or disrupting activity includes anything capable of causing membrane disruption (which may be termed “membrane lysers” (i.e. domains which are capable of lysing a membrane), “membrane permeabilisers” (i.e. domains which are capable of permeabilising a membrane), “membrane disrupters” (i.e. domains which are capable of disrupting a membrane) and/or “membrane destabilizers” (i.e. domains which are capable of destabilizing a membrane)). Such a domain is any domain which is capable of permeabilising and/or disrupting a membrane, preferably the outer membrane/outer leaflet. Such a domain may be an antimicrobial peptide (AMP), such as AMPs that are polycationic, hydrophobic, amphipathic, nano peptides, synthetic natural, Synthetic Mimics of Antimicrobial Peptides (SMAMPs), peptidomimetic oligomer, small molecules, polymeric mimics of AMPs, native AMPs, modified AMPs, or combinations thereof. AMPs may be derived from the same or different origins, for example AMPs may be derived from bacteria (e.g. bacteriocins), viruses, animals (including insects and mammals, e.g. human (such as defensins, including alpha and beta defensins)) and/or plants (such as defensins, thionins, α-hairpinins (hairpin-like peptides), hevein-like peptides, knottins, snakins, lipid-transfer proteins, and cyclotides). AMPs may be selected from the group consisting of: Cathelicidin-BF; Ci-MAM-A24 (Ciona-molecule against microbes A 24-residues); Ranalexin; Nigrocine-2; D-Piscidin 1 [I9K], Chain A, Moronecidin; Cecropin A; Protegrin-1, Chain A; SMAP-29, Sheep myeloid antimicrobial peptide; Lactoferricin B (LfcinB); IR2 (IR) 2-Protegrin-1 (8-13)-(RI) 2; (FR) 2-Protegrin-1 (8-13)-(FR) 2; CEM1; LL-37, Cathelicidin antimicrobial peptide preproprotein; Indolicidin, Cathelicidin-4 precursor; Magainin-2, Chain A; Cecropin A2; Cecropin P1; Pleurocidin precursor; Buforin II; Ascaphine 5; Tilapia Piscidin 4 (TP4); Epinecidin-1 prepropeptide; Chrysophsin-1; Piscidin-like peptide; Myxinidin; PAM2 (platypus antimicrobial 2); Pleurocidin-like peptide AP3 (23-45), NRC-13; 3IQ2 Ras GTPase-activating-like protein IQGAP3 (759-781); SSL-25, Variant and derivative from Dermcidin; KK-Temporin-B (G6A), (Chain A, temporinB_KKG6A); Fowlicidin-2 (1-31); E7K_19K_IsCT Chain A, Cytotoxic linear peptide IsCT; PCNP, Polycationic nonapeptide; E7K_IsCT, Chain A, Cytotoxic linear peptide IsCT; and combinations thereof.

The domains with membrane permeabilising, destabilising and/or disrupting activity may have been demonstrated to have activity against Gram-positive or Gram-negative bacteria, but may not have activity against mycobacteria when used in isolation. For example, Ranalexin has activity against Gram-positive and Gram-negative bacteria, but has not been shown to have activity against mycobacteria (as can be seen in the Database of Antimicrobial Activity and Structure of Peptides (DBAASP), see Peptide Card ID: 13797). However, use of Ranalexin as an AMP fused with a LysA and LysB has been shown herein to have activity against mycobacteria. Therefore, in some embodiments, the domain with membrane permeabilising, destabilising and/or disrupting activity may be a domain that does not have activity against mycobacteria in isolation (i.e. not fused to LysA and/or LysB), but exerts antimycobacterial activity following fusion to LysA and LysB.

In some embodiments, the domain may be an AMP that can permeabilise a cell membrane as defined herein, and/or the domain may be an AMP that destabilises (and/or disrupts) a cell membrane as defined herein. Membranes may be destabilised and/or disrupted (e.g. with an AMP) through chelating or competitively displacing the divalent cations that stabilise the membrane.

Such a domain may be a holin. By “Holins” we include proteins capable of forming pores in a host cell membrane. Holins are small proteins produced by dsDNA bacteriophages which are capable of triggering and controlling the permeabilization of a cell membrane. Holins form pores in the cell membrane, which can facilitate access for enzymes (such as those described for the first and second domains, above, e.g. lysins) to access other cell wall components, such as the peptidoglycan layer and/or other components of the cell wall. The other domains are then able to act on their respective targets.

By “Holins” we include Class I holins, Class II holins and Class III holins, and members of all seven (I to VII) Holin superfamilies. We also include pinholins. Also included are portions of holins that retain their required activity. For example, a holin of the present invention may be a holin or a portion thereof. For example, a portion thereof may be a transmembrane domain of holins, which may have a similar function as the full protein length. Pinholins form small heptameric pores that collapse the membrane potential (the PMF) across the inner membrane, while the more conventional holins form large multi-subunit pores of variable sizes. Holins are classified according to the topology of the seven superfamilies, and according to the mechanism into holins and pinholins.

Such a domain may be a spanin, such as those defined in Kongari et al., 2018 and available in the CPT Spanin Database (https://cpt.tamu.edu/spanindb/#/phages). By “spanin” we include bacteriophage peptides that span the outer membrane of gram-negative bacteria. Spanins include two-component spanins and unimolecular spanins. Prototype two-components spanins include the Rz-Rz1 from phage lambda comprising an integral inner membrane protein (i-spanin) and an outer membrane lipoprotein (o-spanin).

Such a domain may be a cationic or amphipatic domain present in the N- or C-terminus of a bacteriophage lysin. This domain may adopt a helix, random coil or sheet conformation and interact without the outer membrane. This domain may take over the role of a spanin in spanin-less phages. Such a domain may be selected from Lys1521, OBPgp279, PlyE146, EndoT5, SPN9CC, CfP1, LysAB2, LysAB3, LysAB4, PlyAB1, PlyF307, LysABP-01, ABgp46, LysPA26, LysAm24, LysECD7 and/or LysSi3 (Gutierrez Fernandez and Briers, 2020).

In one embodiment, the amino acid sequence of the domain with membrane permeabilising, destabilising and/or disrupting activity may be selected from any one or more of SEQ ID NOs: 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102; or may be encoded by any one or more of the nucleotide sequences of SEQ ID NO: 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99 or 101; as follows:

Length: 678
Type: DNA
Other information: Trehalose dimycolate hydrolase (TDMH)
Sequence ID No: 33
ATGACCATGGCCATCGTTATCTCCCTGCGTAAACCAGCGCGCTTAGCGGCTTTTGCTAGCGCGGTTTTATGTGCG
GGAGCGGCCTTAGCTACCGGTCCTGCTCCAGTTGCGTCTGCCGAACCGTGTAGTGATATTGAAGTCGTTTTCGCC
CGTGGTACCAGCGAGCCTGCAGGAATTGGGCGTGTGGGTCAAGCACTCACGGACGCCATTCGTAACCAAGTAGGC
GGTCGTACCGTGAGTACATATGGTGTAAATTATCCTGCGACTTATGATTTTCTTGCTGCCGCTGATGGTGCGAAT
GATGCTACGAATCGTATTGCCACATTAGCGGAACAATGTCCAAGTACCCGTGTGGTCTTAGGCGGGTATAGTCAA
GGTGCAGCAGTAGTAGATATGCTCTTAGGCATTCCACCGCTCGGGAATAAAGTTGGTAATTTTGGCAGTGCACCC
CCATTGCCTTCGAATCTGATGAATAATGTAGCAGCAGTGGCCGTCTTTGGTAATCCTTCTGCGAAATTTGGGATT
CCTGTGACATCGCGCTTTGGTGGTCGTGCCATTGATGCATGTTCAGATGGAGATCCCATTTGCAGCGATGGCCGT
AATCCATTTGCCCATACACACTATGAATCAAGCCCATTTATTCCTCAAGCGGCCGGCTTAATTGCCGGCTTAGTA
TGA
Length: 225
Molecular weight: 22.68 kDa
Type: protein
Feature: contains the following domains:
Signal peptide Gram-positive entry; 1→39
Cutinase; 41→212
Other information: Trehalose dimycolate hydrolase (TDMH)
Sequence ID No: 34
MTMAIVISLRKPARLAAFASAVLCAGAALATGPAPVASAEPCSDIEVVFARGTSEPAGIGRVGQALTDAIRNQVG
GRTVSTYGVNYPATYDFLAAADGANDATNRIATLAEQCPSTRVVLGGYSQGAAVVDMLLGIPPLGNKVGNFGSAP
PLPSNLMNNVAAVAVFGNPSAKFGIPVTSRFGGRAIDACSDGDPICSDGRNPFAHTHYESSPFIPQAAGLIAGLV
Length: 90
Type: DNA
Organism: Snake venom of Bungarus fasciatus
Other information: Cathelicidin-BF
Sequence ID No: 35
AAATTTTTTCGGAAACTGAAAAAGTCCGTGAAAAAGCGCGCGAAAGAATTTTTCAAAAAGCCGCGGGTAATCGGC
GTGTCCATTCCTTTT
Length: 30
Molecular weight: 3.63 kDa
Type: Protein
Organism: Snake venom of Bungarus fasciatus
Feature: Helix
Other information: Cathelicidin-BF
Sequence ID No: 36
KFFRKLKKSVKKRAKEFFKKPRVIGVSIPF
Length: 72
Type: DNA
Organism: Haemocytes of Ciona intestinalis
Other information: Ci-MAM-A24 (Ciona-molecule against microbes A
24-residues)
Sequence ID No: 37
TGGCGCTCCCTGGGTCGTACGCTGTTACGTCTGTCCCATGCTCTGAAACCGCTGGCGCGCCGTTCCGGGTGG
Length: 24
Molecular weight: 2.83 kDa
Type: Protein
Organism: Haemocytes of Ciona intestinalis
Feature: Helix, cationic
Other information: Ci-MAM-A24 (Ciona-molecule against microbes A
24-residues)
Sequence ID No: 38
WRSLGRTLLRLSHALKPLARRSGW
Length: 61
Type: DNA
Organism: skin of Rana catesbeiana (Bull frog)
Other information: Ranalexin
Sequence ID No: 39
TTTTTGGGAGGCTTAATAAAGATCGTTCCAGCAATGATTTGTGCAGTGACCAAAAAATGTT
Length: 20
Molecular weight: 2.1 kDa
Type: Protein
Organism: skin of Rana catesbeiana (Bull frog)
Feature: helix
Other information: Ranalexin
Sequence ID No: 40
FLGGLIKIVPAMICAVTKKC
Length: 62
Type: DNA
Organism: skin of Pelophylax nigromaculatus (dark-spotted frog)
Other information: Nigrocine-2
Sequence ID No: 41
GGTCTCCTGTCCAAAGTACTGGGCGTTGGCAAAAAAGTGCTGTGCGGTGTCTCTGGTCTGGT
Length: 21
Molecular weight: 2.03 kDa
Type: Protein
Organism: skin of Pelophylax nigromaculatus (dark-spotted frog)
Feature: Amphipathic, α-helix
Other information: Nigrocine-2
Sequence ID No: 42
GLLSKVLGVGKKVLCGVSGLC
Length: 66
Type: DNA
Organism: Mast cells of Morone saxatilis
Other information: D-Piscidin 1 [I9K], Chain A, Moronecidin
Sequence ID No: 43
TTCTTCCATCATATTTTCCGCGGCAAAGTCCATGTGGGCAAGACCATTCATCGCTTAGTAACGGGT
Length: 22
Molecular weight: 2.58 kDa
Type: Protein
Organism: Mast cells of Morone saxatilis
Feature: Helix
Other information: D-Piscidin 1 [I9K], Chain A, Moronecidin
Sequence ID No: 44
FFHHIFRGKVHVGKTIHRLVTG
Length: 120
Type: DNA
Organism: Drosophila melanogaster
Other information: Cecropin A
Sequence ID No: 45
GGCTGGCTGAAAAAAATTGGCAAAAAAATCGAACGCGTGGGCCAGCACACGCGTGATGCAACCATCCAGGGTCTG
GGTATCCCACAGCAGGCAGCTAACGTAGCCGCGACTGCTCGTGGT
Length: 40
Molecular weight: 4.23 kDa
Type: Protein
Organism: Drosophila melanogaster
Feature: cationic, helical
Other information: Cecropin A
Sequence ID No: 46
GWLKKIGKKIERVGQHTRDATIQGLGIPQQAANVAATARG
Length: 54
Type: DNA
Organism: Leukocytes of Sus scrofa
Other information: Protegrin-1, Chain A
Sequence ID No: 47
CGTGGCGGTCGTCTGTGTTACTGCCGTCGCCGTTTCTGCGTGTGTGTTGGTCGT
Length: 18
Molecular weight: 2.16 kDa
Type: Protein
Organism: Leukocytes of Sus scrofa
Feature: Cyclic, Cationic
Other information: Protegrin-1, Chain A
Sequence ID No: 48
RGGRLCYCRRRFCVCVGR
Length: 87
Type: DNA
Organism: Leukocytes of Ovis aries
Other information: SMAP-29, Sheep myeloid antimicrobial peptide
Sequence ID No: 49
CGTGGTCTGCGTCGCCTGGGTCGCAAAATTGCGCACGGCGTCAAAAAATACGGCCCGACCGTGCTGCGCATTATC
CGCATCGCTGGT
Length: 29
Molecular weight: 3.25 kDa
Type: Protein
Organism: Leukocytes of Ovis aries
Feature: helical
Other information: SMAP-29, Sheep myeloid antimicrobial peptide
Sequence ID No: 50
RGLRRLGRKIAHGVKKYGPTVLRIIRIAG
Length: 45
Type: DNA
Organism: Bos taurus
Other information: Lactoferricin B (LfcinB)
Sequence ID No: 51
TTTAAATGTCGTCGCTGGCAATGGCGTATGAAAAAACTGGGGGCG
Length: 15
Molecular weight: 1.994 kDa
Type: Protein
Organism: Bos taurus
Feature: Beta, cationic
Other information: Lactoferricin B (LfcinB)
Sequence ID No: 52
FKCRRWQWRMKKLGA
Length: 42
Type: DNA
Organism: synthetic variant of Protegrin-1
Other information: IR2 (IR) 2-Protegrin-1 (8-13)-(RI) 2
Sequence ID No: 53
ATCCGGATCCGTTGCCGTCGCCGGTTCTGCCGCATTCGTATT
Length: 14
Molecular weight: 2.41 kDa
Type: Protein
Organism: synthetic variant of Protegrin-1
Feature: helix
Other information: IR2 (IR) 2-Protegrin-1 (8-13)-(RI) 2
Sequence ID No: 54
IRIRCRRRFCRIRI
Length: 42
Type: DNA
Organism: synthetic variant of Protegrin-1
Other information: (FR) 2-Protegrin-1 (8-13)-(FR) 2
Sequence ID No: 55
TTCCGCTTTCGTTGTCGCCGGCGTTTTTGTCGGTTCCGCTTC
Length: 14
Molecular weight: 2.05 kDa
Type: Protein
Organism: synthetic variant of Protegrin-1
Feature: helix
Other information: (FR) 2-Protegrin-1 (8-13)-(FR) 2
Sequence ID No: 56
FRFRCRRRFCRFRF
Length: 36
Type: DNA
Organism: synthetic variant of MSI-78 (1-12)
Other information: CEM1
Sequence ID No: 57
GGCATTGGTAAGTTCCTGAAAAAAGCGAAGAAATTT
Length: 12
Molecular weight: 1.364 kDa
Type: Protein
Organism: synthetic variant of MSI-78 (1-12)
Feature: helix
Other information: CEM1
Sequence ID No: 58
GIGKFLKKAKKF
Length: 111
Type: DNA
Organism: Neutrophil specific granules of Homo sapiens
Other information: LL-37, Cathelicidin antimicrobial peptide preproprotein
Sequence ID No: 59
CTCCTGGGTGACTTCTTTCGCAAATCCAAAGAGAAAATCGGCAAAGAGTTCAAACGTATCGTGCAGCGCATTAAA
GACTTTCTGCGTAACCTGGTTCCGCGTACCGAATCT
Length: 37
Molecular weight: 3.25 kDa
Type: Protein
Organism: Neutrophil specific granules of Homo sapiens
Feature: Amphipathic, helical
Other information: LL-37, Cathelicidin antimicrobial peptide preproprotein
Sequence ID No: 60
LLGDFFRKSKEKIGKEFKRIVQRIKDFLRNLVPRTES
Length: 39
Type: DNA
Organism: Neutrophils of Bos taurus [Bovine]
Other information: Indolicidin, Cathelicidin-4 precursor
Sequence ID No: 61
ATCCTGCCGTGGAAATGGCCGTGGTGGCCATGGCGTCGC
Length: 13
Molecular weight: 1.907 kDa
Type: Protein
Organism: Neutrophils of Bos taurus [Bovine]
Other information: Indolicidin, Cathelicidin-4 precursor
Sequence ID No: 62
ILPWKWPWWPWRR
Length: 69
Type: DNA
Organism: skin and Stomach of African clawed frog Xenopus laevis
Other information: Magainin-2, Chain A
Sequence ID No: 63
GGTATCGGCAAATTCCTGCACTCCGCAAAAAAATTCGGCAAAGCTTTCGTGGGCGAAATTATGAACTCT
Length: 23
Molecular weight: 2.46 kDa
Type: Protein
Organism: skin and Stomach of African clawed frog Xenopus laevis
Feature: Helical, cationic
Other information: Magainin-2, Chain A
Sequence ID No: 64
GIGKFLHSAKKFGKAFVGEIMNS
Length: 108
Type: DNA
Organism: Aedes aegypti
Other information: Cecropin A2
Sequence ID No: 65
GGCGGCCTGAAAAAACTGGGCAAAAAACTGGAAGGTGCCGGCAAACGTGTGTTCAACGCTGCAGAAAAAGCACTG
CCGGTTGTAGCTGGTGCTAAAGCTCTCCGTAAA
Length: 36
Molecular weight: 3.67 kDa
Type: Protein
Organism: Aedes aegypti
Feature: Helix
Other information: Cecropin A2
Sequence ID No: 66
GGLKKLGKKLEGAGKRVFNAAEKALPVVAGAKALRK
Length: 93
Type: DNA
Organism: Ascaris suum
Other information: Cecropin P1
Sequence ID No: 67
TCTTGGCTGAGCAAAACCGCGAAAAAACTGGAGAACTCCGCCAAAAAACGCATCTCTGAAGGCATTGCTATTGCA
ATCCAGGGTGGTCCGCGT
Length: 31
Molecular weight: 3.67 kDa
Type: Protein
Organism: Ascaris suum
Feature: Helix
Other information: Cecropin P1
Sequence ID No: 68
SWLSKTAKKLENSAKKRISEGIAIAIQGGPR
Length: 75
Type: DNA
Organism: the skin mucous secretions of Winter flounder Pleuronectes
americanus
Other information: Pleurocidin precursor
Sequence ID No: 69
GGCTGGGGTTCTTTCTTTAAAAAAGCGGCTCACGTTGGCAAACATGTAGGTAAAGCAGCTCTGACCCACTATCTG
Length: 25
Molecular weight: 2.71 kDa
Type: Protein
Organism: the skin mucous secretions of Winter flounder Pleuronectes
americanus
Feature: Helix
Other information: Pleurocidin precursor
Sequence ID No: 70
GWGSFFKKAAHVGKHVGKAALTHYL
Length: 63
Type: DNA
Organism: derivative of buforin I (from stomach of Bufo bufo) after
treatment with endoproteinase
Other information: Buforin II
Sequence ID No: 71
ACCCGTAGCTCTCGTGCTGGCCTGCAGTTTCCGGTTGGTCGCGTGCACCGTCTGCTCCGCAAA
Length: 21
Molecular weight: 2.43 kDa
Type: Protein
Organism: derivative of buforin I (from stomach of Bufo bufo) after
treatment with endoproteinase
Feature: Helix, cationic
Other information: Buforin II
Sequence ID No: 72
TRSSRAGLQFPVGRVHRLLRK
Length: 72
Type: DNA
Organism: skin of Ascaphus truei (tailed frog)
Other information: Ascaphine 5
Sequence ID No: 73
GGTATCAAAGATTGGATCAAAGGCGCAGCGAAAAAACTGATCAAAACCGTTGCCTCTCACATTGCTAACCAG
Length: 24
Molecular weight: 2.59 kDa
Type: Protein
Organism: skin of Ascaphus truei (tailed frog)
Feature: helix
Other information: Ascaphine 5
Sequence ID No: 74
GIKDWIKGAAKKLIKTVASHIANQ
Length: 75
Type: DNA
Organism: gills of Nile Tilapia, Oreochromis niloticus
Other information: Tilapia Piscidin 4 (TP4)
Sequence ID No: 75
TTTATCCATCACATTATTGGTGGTCTGTTCTCCGCAGGTAAAGCAATTCATCGCCTGATTCGGCGTCGTCGGCGC
Length: 25
Molecular weight: 2.98 kDa
Type: Protein
Organism: gills of Nile Tilapia, Oreochromis niloticus
Other information: Tilapia Piscidin 4 (TP4)
Sequence ID No: 76
FIHHIIGGLESAGKAIHRLIRRRRR
Length: 75
Type: DNA
Organism: orange-spotted grouper, Epinephelus coioides
Other information: Epinecidin-1 prepropeptide
Sequence ID No: 77
TTTATCTTTCACATTATTAAAGGTCTGTTCCATGCGGGGAAAATGATCCACGGCTTAGTGACCCGGCGTCGCCAT
Length: 25
Molecular weight: 2.98 kDa
Type: Protein
Organism: orange-spotted grouper, Epinephelus coioides
Other information: Epinecidin-1 prepropeptide
Sequence ID No: 78
FIFHIIKGLFHAGKMIHGLVTRRRH
Length: 75
Type: DNA
Organism: the pyloric caeca and gills of Red sea bream Chrysophrys
major or Pagrus major
Other information: Chrysophsin-1
Sequence ID No: 79
TTCTTTGGATGGCTTATCAAAGGCGCAATTCATGCTGGGAAAGCGATTCATGGCCTTATCCATCGCCGTCGTCAT
Length: 25
Molecular weight: 2.89 kDa
Type: Protein
Organism: the pyloric caeca and gills of Red sea bream, Chrysophrys
major or Pagrus major
Feature: Helix
Other information: Chrysophsin-1
Sequence ID No: 80
FFGWLIKGAIHAGKAIHGLIHRRRH
Length: 66
Type: DNA
Organism: Spleen of Larimichthys crocea (large yellow croaker)
Other information: Piscidin-like peptide
Sequence ID No: 81
ATCTGGGGCTTAATCGCGCACGGGGTGGGTCATGTTGGACGTCTGATTCATGGCCTGATTCGCGGT
Length: 22
Molecular weight: 2.33 kDa
Type: Protein
Organism: Spleen of Larimichthys crocea (large yellow croaker)
Feature: Helix
Other information: Piscidin-like peptide
Sequence ID No: 82
IWGLIAHGVGHVGRLIHGLIRG
Length: 36
Type: DNA
Organism: Epidermal mucus of Myxine glutinosa
Other information: Myxinidin
Sequence ID No: 83
GGTATTCATGATATCCTGAAATATGGCAAACCGAGC
Length: 12
Molecular weight: 1.32 kDa
Type: Protein
Organism: Epidermal mucus of Myxine glutinosa
Feature: Helix
Other information: Myxinidin
Sequence ID No: 84
GIHDILKYGKPS
Length: 66
Type: DNA
Organism: Ornithorhynchus anatinus
Other information: PAM2 (platypus antimicrobial 2)
Sequence ID No: 85
CGCCCGTGGGCGGGCAACGGCAGCGTGCATCGCTATACCGTGCTGAGCCCGCGCCTGAAAACCCAG
Length: 22
Molecular weight: 2.52 kDa
Type: Protein
Organism: Ornithorhynchus anatinus
Other information: PAM2 (platypus antimicrobial 2)
Sequence ID No: 86
RPWAGNGSVHRYTVLSPRLKTQ
Length: 69
Type: DNA
Organism: Hippoglossoides platessoides
Other information: Pleurocidin-like peptide AP3 (23-45) , NRC-13
Sequence ID No: 87
GGCTGGCGCACCCTGCTGAAAAAAGCGGAAGTGAAAACCGTGGGCAAACTGGCGCTGAAACATTATCTG
Length: 23
Molecular weight: 2.65 kDa
Type: Protein
Organism: Hippoglossoides platessoides
Other information: Pleurocidin-like peptide AP3 (23-45) , NRC-13
Sequence ID No: 88
GWRTLLKKAEVKTVGKLALKHYL
Length: 69
Type: DNA
Organism: Synthetic construct from Human cytoskeletal scaffolding proteins
IQGAP3
Other information: 31Q2 Ras GTPase-activating-like protein IQGAP3 (759-781)
Sequence ID No: 89
ACCTGGCTCCCAGCAGTAATTAAGATCCAGGCGCATTGGCGGGGCTATCGTCAGCGTAAGATTTATCTG
Length: 23
Molecular weight: 2.89 kDa
Type: Protein
Organism: Synthetic construct from Human cytoskeletal scaffolding proteins
IQGAP3
Feature: hydrophobic
Other information: 3IQ2 Ras GTPase-activating-like protein IQGAP3 (759-781)
Sequence ID No: 90
TWLPAVIKIQAHWRGYRQRKIYL
Length: 75
Type: DNA
Organism: Eccrine sweat glands, skin, Homo sapiens
Other information: SSL-25, Variant and derivative from Dermcidin
Sequence ID No: 91
AGCTCGCTTCTGGAAAAAGGGTTGGACGGCGCCAAAAAAGCGGTTGGCGGCCTTGGTAAACTGGGGAAAGACGCC
Length: 25
Molecular weight: 2.41 kDa
Type: Protein
Organism: Eccrine sweat glands, skin, Homo sapiens
Feature: helix
Other information: SSL-25, Variant and derivative from Dermcidin
Sequence ID No: 92
SSLLEKGLDGAKKAVGGLGKLGKDA
Length: 45
Type: DNA
Organism: synthetic
Other information: KK-Temporin-B (G6A) , (Chain A, temporinB_KKG6A)
Sequence ID No: 93
AAAAAATTACTGCCCATCGTCGCCAATCTCCTGAAATCCCTTTTA
Length: 15
Molecular weight: 1.663 kDa
Type: Protein
Organism: synthetic
Other information: KK-Temporin-B (G6A) , (Chain A, temporinB_KKG6A)
Sequence ID No: 94
KKLLPIVANLLKSLL
Length: 93
Type: DNA
Organism: Gallus gallus
Other information: Fowlicidin-2 (1-31)
Sequence ID No: 95
TTAGTACAGCGCGGGCGTTTTGGTCGTTTCCTGCGCAAAATCCGTCGTTTTCGTCCAAAAGTCACGATTACGATC
CAGGGTAGTGCCCGTTTT
Length: 31
Molecular weight: 3.76 kDa
Type: Protein
Organism: Gallus gallus
Feature: Helix
Other information: Fowlicidin-2 (1-31)
Sequence ID No: 96
LVQRGRFGRFLRKIRRFRPKVTITIQGSARF
Length: 39
Type: DNA
Organism: Scorpion venom of Opisthacanthus madagascariensis
Other information: E7K 19K IsCT Chain A, Cytotoxic linear peptide IsCT
Sequence ID No: 97
ATTCTGGGCAAAATTTGGAAAGGCAAAAAAAGCCTGTTT
Length: 13
Molecular weight: 1.517 kDa
Type: Protein
Organism: Scorpion venom of Opisthacanthus madagascariensis
Feature: Helix
Other information: E7K I9K IsCT, Chain A, Cytotoxic linear peptide IsCT
Sequence ID No: 98
ILGKIWKGKKSLF
Length: 27
Type: DNA
Organism: Synthetic
Other information: PCNP, Polycationic nonapeptide
Sequence ID No: 99
AAACGCAAGAAACGTAAGAAACGCAAA
Length: 9
Molecular weight: 1.255 kDa
Type: Protein
Organism: Synthetic
Feature: Polycationic
Other information: PCNP, Polycationic nonapeptide
Sequence ID No: 100
KRKKRKKRK
Length: 39
Type: DNA
Organism: Scorpion venom of Opisthacanthus madagascariensis
Other information: E7K IsCT, Chain A, Cytotoxic linear peptide IsCT
Sequence ID No: 101
ATTCTGGGCAAAATTTGGAAAGGCATTAAAAGCCTGTTT
Length: 13
Molecular weight: 1.502 kDa
Type: Protein
Organism: Scorpion venom of Opisthacanthus madagascariensis
Feature: Helix
Other information: E7K IsCT, Chain A, Cytotoxic linear peptide IsCT
Sequence ID No: 102
ILGKIWKGIKSLF

As discussed above, the peptides of the present invention (comprising the three domains as outlined above) are capable of targeting cell envelopes and acting on these to exert an effect on the membrane as well as the cell wall. Such peptides may permeabilise the membrane and break down the cell wall to disrupt bacteria and exert an antibacterial effect.

Membranes/cell walls of interest for the present invention (e.g. membranes targeted by peptides of the present invention) are comprised of multiple components (see FIG. 1). Target membranes (or target organisms comprising such membranes) may comprise any one or more of the following components: (i) an outer leaflet (also known as the outer membrane); (ii) an inner leaflet; (iii) an arabinogalactan layer; and/or (iv) a peptidoglycan layer. Target membranes (or organisms) may further comprise: (v) a periplasmic space; (vi) a granular layer; and/or (vii) an inner membrane. By “cell envelope” we also include the meaning of the equivalent terms “cell wall” and “cell membrane”.

The compositions of the layers are as described in Vincent et al., 2018. The granular layer is between the peptidoglycan layer and plasma membrane, which may be linked to the plasma membrane and composed of penicillin-binding proteins, lipoproteins, and lipoteichoic acids (or teichuronic acid in some species). The peptidoglycan layer is comprised of short peptides and glycan strands that are composed of N-acetylglucosamine and N-acetylmuramic acid residues linked by β-1→4 bonds. The arabinogalactan layer may also be attached to the peptidoglycan layer (such as via covalent links to the N-acetylmuramic acid residues and Lcp1). The inner leaflet of the external mycomembrane is homogeneous and mainly composed of mycolic acids (MAS). These MAs are long chain fatty acids that are exclusive to the order Corynebacteriales, and can form a barrier to hydrophilic molecules, including some antibiotics. The outer leaflet is highly heterogeneous and consists of lipids (such as phthiocerol dimycocerosate (PDIM), phenolic glycolipid (PGL, phenolphthiocerol-based glycolipids that share a similar long-chain fatty acid backbone with PDIM), and lipooligosaccharides (LOS)), lipoglycans and proteins. The outer leaflet may also contain trehalose monomycolate (TMM) and trehalose dimycolate (TDM), which consist of glucose disaccharides (α-D-glucopyranosyl-α-D-glucopyranoside) esterified with MAs.

Accordingly, peptides of the present invention may be defined by the type of membrane they are capable of targeting. In one embodiment the peptide is capable of targeting a mycobacterial cell envelope (including a cell wall/cell membrane, for example, the mycomembrane). Membranes of interest may also be defined by their thickness. For example, a mycobacterial cell envelope is typically made up of: (i) an outer leaflet and inner leaflet of about 7.5 nm (collectively also known as the mycomembrane); (ii) arabinogalactan and peptidoglycan layers of about 6.3 nm (total); a periplasmic space of about 14.1 nm; a granular layer of about 3.8 nm; and an inner membrane of about 6.3 nm. Thus, a typical mycobacterial cell envelope is a total of about 38 nm. Mycobacterial strains with drug resistance may have a thicker cell envelope. Velayati et al., 2009 demonstrated through transmission electron microscopy (TEM) that there are marked differences in the thickness of cell envelopes between extensively drug-resistant (XDR; 20.2±1.5 nm), multidrug-resistant (MDR; 17.1±1.03 nm) and susceptible tuberculosis (TB; 15.6±1.3 nm) bacilli.

Thus, peptides of the present invention may be peptides capable of causing lysis of a membrane that is at least 10 nm, 15 nm, 20 nm, 25 nm, 30 nm, 35 nm, 40 nm, 45 nm, 50 nm or more in thickness.

The outer leaflet and inner leaflet may collectively be termed the “outer membrane”. Therefore, domains of the present invention that have membrane permeabilising activity may be outer membrane permeabilisers that may target the outer membrane (or component thereof). Membrane permeabilisers may target an inner membrane (or component thereof). Membrane permeabilisers may target the outer leaflet, inner leaflet, both the outer and inner leaflet, and/or the inner membrane. For example, membrane permeabilisers may target the inner leaflet of the outer membrane and the inner membrane, but not the outer leaflet of the outer membrane. Alternatively, membrane permeabilisers may target only the outer leaflet of the outer membrane or only the inner leaflet of the outer membrane. Alternatively, membrane permeabilisers may target each leaflet of the outer membrane and the inner membrane.

The targeting of the outer leaflet, inner leaflet and/or inner membrane may be achieved by using multiple membrane permeabilisers. For example, a first membrane permeabiliser that targets the outer leaflet may be used in combination (for example, as a fusion peptide) with a second membrane permeabiliser that targets the inner leaflet. Alternatively, or additionally, a first membrane permeabiliser may target the outer leaflet, inner leaflet and inner membrane, and at least one further membrane permeabiliser is included that targets any one or more of these layers. Combinations of membrane permeabilisers are contemplated that may work additively or synergistically with each other. Accordingly, the requirement to target the outer leaflet, inner leaflet and inner membrane (if required for the target cell membrane and/or organism comprising said membrane) may be built up (and/or enhanced) by including multiple membrane permeabilisers, whether as multiple copies of the same membrane permeabilisers or as multiple different membrane permeabilisers.

In some embodiments, the membrane permeabiliser is an antimicrobial peptide (AMP) or portion thereof. The AMP or a portion thereof may be cationic, polycationic, hydrophobic, amphipathic, synthetic, natural, native or modified, or any combination thereof, whether from different origins (for example, bacteria, fungi, viruses, animals, insects, humans) or the same origin. For example, an AMP may be cationic. Alternatively, an AMP may be cationic in a portion and amphipathic in a different portion of the same AMP. Alternatively, or additionally, multiple AMPs with the same or different properties may be present in a peptide of the present invention.

In some embodiments, the membrane permeabiliser is associated with a non-AMP mechanism, for example the membrane permeabiliser may be at least one holin, as described above. Holins and AMPs may be used in combination.

A domain (e.g. a fourth domain) may correspond to a protein transduction domain (PTD). These may also be referred to as protein transducing domains. Such domains may be added to peptides of the present invention to facilitate transduction (e.g. protein/peptide transduction). By “transduction” we include the meaning that internalization is facilitated, or allowing the passage across a cell membrane or portion thereof. Further, a PTD may also be classified as a cell-penetrating peptide (CPP), which may be capable of targeting intracellularly components, including intracellular proteins. For example, a peptide further comprising a fourth domain that is at least one PTD/CPP (use of “PTD” herein is to be construed as also including “CPP”) may have improved transduction efficiency compared with the same peptide lacking such a domain. Key benefits to a PTD domain include, but are not limited to, improved efficacy of fusion proteins, improved transduction efficiency, improved safety, and/or improved stability. The targeting of mycobacteria by peptides may be facilitated by at least one PTD if the mycobacteria are present inside of cells, such as macrophages. Example PTDs include those described in Bechara and Sagan, 2013. For example, the PTD may be protein-derived (such as Penetratin, Tat peptide, pVEC); chimeric (such as Transportan, MPG, Pep-1); and/or synthetic (such as Polyarginines, MAP, R₆W₃). Accordingly, in some embodiments, one or more PTD is selected from the group consisting of Penetratin, Tat peptide, pVEC, Transportan, MPG, Pep-1, Polyarginines, MAP, R₆W₃. In some embodiments, the PTDs are cationic poly-Arg containing PTDs, for example PTD3 and/or TAT.

Any of the above that references a particular domain is intended to also be reference to a fragment (e.g. a truncated version), variant (which includes mutants), fusion or derivative thereof, or a fusion of said fragment, variant or derivative thereof of said domain, in addition to any wildtype domains. For example, at least one membrane permeabiliser may be a fragment (e.g. a fragment with substantially the same function) of a membrane permeabiliser, combined with at least one wildtype LysA, at least one variant LysB and at least one wildtype PTD. Any of these domains (if present in the fusion protein) may be presented as any “version” (i.e. fragment, variant, fusion or derivative thereof, or a fusion of said fragment, variant or derivative thereof, wildtype) without limitation to the version of any other domain that is present. Any domains described herein may be natural (i.e. wildtype) or synthetic domains.

Any domain referred to herein may be a fragment, variant, fusion or derivative thereof, or a fusion of said fragment, variant or derivative thereof. In one embodiment, the variant has an amino acid sequence which has at least 50% identity with the amino acid sequence according to a reference sequence (for example, the amino acid sequences of SEQ ID NOS: 1-102, or SEQ ID NOs: 326 to 338), or a fragment thereof, for example at least 55%, 60%, 65%, 70%, 75%, 80%, 85%, 90%, 95%, 96%, 97%, 98% or at least 99% identity, optionally wherein the fragment, variant, fusion or derivative thereof, or a fusion of said fragment, variant or derivative thereof retains substantially the same level of activity of the wildtype domain. Substantially the same level of activity may be at least 10%, 20%, 30%, 40%, 50%, 60%, 70%, 80%, 90%, 95%, 96%, 97%, 98%, 99%, 100% or higher activity compared with the wildtype domain.

In some embodiments, the peptide corresponds to the amino acid sequence (or a nucleic acid sequence that encodes the amino acid sequence) according to any one or more of SEQ ID NOs: 147-311, or a fragment, variant, fusion or derivative thereof, or a fusion of said fragment, variant or derivative thereof. In one embodiment, the variant has an amino acid sequence which has at least 50% identity with the amino acid sequence (or a nucleic acid sequence that encodes the amino acid sequence) according to SEQ ID NOS: 147-311), or a fragment thereof, for example at least 55%, 60%, 65%, 70%, 75%, 80%, 85%, 90%, 95%, 96%, 97%, 98% or at least 99% identity, optionally wherein the fragment, variant, fusion or derivative thereof, or a fusion of said fragment, variant or derivative thereof retains substantially the same level of activity of the wildtype domain. Substantially the same level of activity may be at least 10%, 20%, 30%, 40%, 50%, 60%, 70%, 80%, 90%, 95%, 96%, 97%, 98%, 99%, 100% or higher activity compared with the wildtype domain.

Accordingly, exemplary active constructs of the present invention may be selected from SEQ ID NOS: 147-311, some of which are included below, as follows:

```
Sequence Id1 (1B4)
  (SEQ ID NO: 147)
ATGCACCATCATCATCATCATTCTTCTGGTGTAGATCTGGGTACCGAGAACCTGTACTTCCAATCCATGCGTGGC
GGTCGTCTGTGTTACTGCCGTCGCCGTTTCTGCGTGTGTGTTGGTCGTGGTGCAAGCAAGCCCTGGCTGTTCACC
GTTCACGGCACGGGCCAGCCCGATCCCCTCGGGCCTGGCCTGCCTGCCGATACGGCACGCGACGTACTTGACATC
TACCGGTGGCAGCCCATCGGCAACTACCCCGCTGCGGCCTTCCCGATGTGGCCGTCGGTCGAGAAGGGTGTCGCC
GAGCTGATCCTGCAGATCGAGCTGAAGCTGGACGCGGACCCCTACGCGGACTTCGCGATGGCGGGTTACTCGCAG
GGAGCCATCGTGGTTGGCCAGGTGCTCAAGCACCACATCCTGCCTCCGACGGGCAGGCTCCACAGGTTCCTGCAC
CGGCTCAAGAAGGTCATCTTCTGGGGTAATCCCATGCGGCAGAAGGGCTTTGCCCACTCTGACGAGTGGATCCAC
CCGGTCGCTGCCCCTGACACCCTCGGAATCCTCGAGGACCGGCTCGAAAACCTGGAGCAGTACGGCTTCGAGGTC
CGCGACTACGCCCACGACGGTGACATGTACGCCTCCATCAAAGAGGACGACCTGCACGAATACGAGGTCGCCATC
GGCCGGATCGTGATGAAGGCCAGCGGCTTCATCGGTGGCCGGGACTCCGTGGTAGCCCAGCTCATCGAGCTTGGC
CAGCGTCCGATCACCGAGGGAATTGCGTTGGCGGGAGCCATCATCGACGCCCTCACGTTCTTCGCCCGCTCTCGT
ATGGGCGACAAGTGGCCGCACCTCTACAACCGCTACCCGGCGGTCGAGTTCCTACGACAGATCGCAGGTCCAGCC
GTTCCACCTCCGGGAAGCTCATTTACGCGCTTTCTCCAAGACGATCCCTTGCTGACTCGTGAACAGGTAATGGCA
GAACTCATCCGCGTTGCTGATGATGAATTGAATATGCCTGATAAACGTGGAGCATGTGTGATCGCAGGAATGACT
ATCTCACAGGAAGTTGGTGTGAAAGATAATGATCCCCCATTTGAACGCCGTTTCTGGTGTCCAGCAAATCGTGCG
GATCCAGAGTCTTTTAATTATCCACATGATAGTGAGTCCAATGATGGACGTTCAGTGGGGTATTTTCAACAACAA
AAGGGCCCAAATGGTGAACTTTGGTGGGGCACTACCGCGAGTGAAATGAATTTACATAGTGCAGCCACCCAATTC
ATGACTCGTTTAAAAGCCGCGGGTTATAATGCCTCTAATGCGCAAGCCGCAAATGATTCTGCTCAAGCTATTCAA
CGTTCAGGTGTTCCACAAGCCTATAAACAGTGGTGGGATGATATCAATCGTTTATATGATAAAGTAAAAGGTAGC
GGCGGCGGACCCGCACCGGCTCCCAAACCACCTCAAAGCGGTCCTTGGACTGGTGATCCTGTTTGGTTAGCAGAT
GTATTACGTGCAGAAGGCTTGAATGTTGTTGAACTCCCTGGTTGGTTAGATCGTGGTCATGGAGATATGGGTCGT
CTGTGGGGCGTCGTATGTCACCATACTGGTAGTGACAATACTCCAAGCTCTGAAATCGCATTCCATCCCAGCCTG
GGTCTTTGTTCCCAAATCCATCTTGCCCGTAATGGTACGGTTACACTTTGTGGCGTTGGTATTGCGTGGCACGCA
GGAGTGGGATCGTACCCGGGGTTGCCGGAAGATAATGCAAATGCCGTGACAATTGGTATTGAAGCACAAAATTCG
GGTACATACGATGGTGCTCCTCATCGCACCAACTGGCCGGATGCACAGTATGATGCGTACGTCAAATGTTGTGCG
GCTATTTGTCGTCGTCTGGGAGTCCGTGCAGACCATGTAATTTCGCATAAAGAGTGGGCGGGCCGTAAACAGGGT
AAGTGGGACCCGGGAGCGATTGATATGAATATTTTCCGTGCGGATGTTCAACGTCGTATTGATGCGCACCAGCCG
AATGGTGAAGATGACTTTATGGCGGCTCTGAGCGCGGATGAACAACGTGAAGTTTTGAATCTTTTGCGTGTTTTA
GCGGATCGCCGCTTTGTGAGTCGTTCACCTTTTCGTCATCTCGGTGAAGGTCCATCCGAGACGGTAGCGGGCTTT
GGTTTAAATACGGATGGACTGAACCATGCTCAATATACCATCGAACTCGCACGTCTCGGGGATCCTACCCATTTA
GCGTTACTCCGTGAAGTAGCTAGTGCGGAAGGGGATTCTCGTTACCCGGATCGCCAATATGATGCGAAATTGGCG
AAACGTGTCTTGGCTGAGATTGAAGGTGCGGCGACCGCGCCTGCGAAACCTTCTACTCCTTCAGCGCCAACGGAA
CCAGCACCGGAAGCACCTACTCCACCGGTGAAAGCGGCTTGTGCACTCAGCGCCGCGGGCTGTGTAGTAGCGGGG
AGCACTTCAGGTGGTGGTTGTGCGTTATCGACAGATGGTACGGGGAAATGTGTGGTCACTGCGGCCACTGATGGT
GGCGCGGCAGGCTCATAATAGTGATAATAG
Sequence Id2
  (SEQ ID NO: 148)
MHHHHHHSSGVDLGTENLYFQSMRGGRLCYCRRRFCVCVGRGASKPWLFTVHGTGQPDPLGPGLPADTARDVLDI
YRWQPIGNYPAAAFPMWPSVEKGVAELILQIELKLDADPYADFAMAGYSQGAIVVGQVLKHHILPPTGRLHRELH
RLKKVIFWGNPMRQKGFAHSDEWIHPVAAPDTLGILEDRLENLEQYGFEVRDYAHDGDMYASIKEDDLHEYEVAI
GRIVMKASGFIGGRDSVVAQLIELGQRPITEGIALAGAIIDALTFFARSRMGDKWPHLYNRYPAVEFLRQIAGPA
VPPPGSSFTRFLQDDPLLTREQVMAELIRVADDELNMPDKRGACVIAGMTISQEVGVKDNDPPFERREWCPANRA
DPESFNYPHDSESNDGRSVGYFQQQKGPNGELWWGTTASEMNLHSAATQFMTRLKAAGYNASNAQAANDSAQAIQ
RSGVPQAYKQWWDDINRLYDKVKGSGGGPAPAPKPPQSGPWTGDPVWLADVLRAEGLNVVELPGWLDRGHGDMGR
LWGVVCHHTGSDNTPSSEIAFHPSLGLCSQIHLARNGTVTLCGVGIAWHAGVGSYPGLPEDNANAVTIGIEAQNS
GTYDGAPHRTNWPDAQYDAYVKCCAAICRRLGVRADHVISHKEWAGRKQGKWDPGAIDMNIFRADVQRRIDAHQP
NGEDDFMAALSADEQREVLNLLRVLADRRFVSRSPFRHLGEGPSETVAGFGLNTDGLNHAQYTIELARLGDPTHL
ALLREVASAEGDSRYPDRQYDAKLAKRVLAEIEGAATAPAKPSTPSAPTEPAPEAPTPPVKAACALSAAGCVVAG
STSGGGCALSTDGTGKCVVTAATDGGAAGS
Sequence Id3 (1B7)
  (SEQ ID NO: 149)
ATGCACCATCATCATCATCATTCTTCTGGTGTAGATCTGGGTACCGAGAACCTGTACTTCCAATCCATGAAACGC
AAGAAACGTAAGAAACGCAAAGGTGCATTGAACGGTGAATTTTACGTTCCGGCAGGGACAATCATTACTGCCAAC
GGGACAGGGGTTCCAGATGGTGCGGGGCCCGGATTTGGTGCCGATCTGGCTCGCGAACTGGAGCGTGTGGCTGAA
GGCATGTGGATTTGGAAATGGATCAACTACCCAGCAGCAGTAATGCCGATGCGTCCGAGCATTAATACGTTGCGT
TTGGCTTTAAAAGAGATGATCCGTACCACTCCGGGCAAAATTGTTTTATCGGCGTACTCTCAATCGGCCGTGGCT
TTTGCTTACGTATGGCGCGACGACATTCTTAACCCCGATGGTGAACTTCACGACCGCCTGGATGATATTGTAGCA
GTAATCTTGTACGGCAACCCAGTTCGCGCCCCTGGGATTGCTTATGGCAACGAGTTAGGCGGTCAATCTGCCCCC
GGCAAGCTGAATGGGCATGTCACCGGCGGCATCGCCGGGCCTGATTGTTTGCGCCCGGAAGAGTGCGTCCATCCC
GTCACTGGGCGTCGCATTGTACTGGACTTCGCTAATGCCGGTGATTTGTACGCTGCTGCACCCGTTGGCGCGGAA
CCCTGGGTCAAAGAGACTGAGGTAGGCCATAACGAGACGCTTATTTACGAAGCTGTTATGGATTTTAATGGCCGC
GATATCCTTGCATTCGCCAAAGAGATCGCACAGATTCTTACAATGCCCTTGAGCCAGGTCGTACCGCTGGTACAA
GCAATTATCAACGGGTTAAGCTTTCTTGTCCAGGGCCCGAAAGCTCCGCACTGGACGTACACGATTGGCCCGGCA
GTCGATTACTTAGTCCGCACGGGCAATGAACTTCGTAAAAATGCAGGTCCGGCGGTTCCGCCGCCGGGAAGCACT
ACAAAGGACCAGGTAGCGCAGATTACAATTGCAGAGGCAAAAGCCCGTGGGTATACTCGTTCAGAGTGTTTAGCC
ATTATGTCTACGTTTTATCAGGAAAGCGGTTGGAATGATACTATTTGGGATCCTACGCATACAACGTATGGAATC
GCGCAACAAGATGGTAGTTATCCCCATCGTTTTGATGGCGCGGCCGCGCAGATTAAGGGTTTCTTTGATAAACTG
GATGTTTGGCGTGCAAAGCCGGGCGCATCGACAGACATTTGGCTTAATATTTGTTGGATGCAACAAGCGCCGAAT
TGGCCGTCGGCCGATTATTGGTATGCGAATGGTCGTCGTGCTTATCTGACAGAGATTAAAAGTCGTATTGCGACG
GTTACGCCGTATCTGGATAAATATTGGCCTGCTGACGGCGGCACGGCGGTTCCAGATGAGCCTCGTCCGGACTTT
AATGAGTTCCCAATTTGGTCAAATAATAATTCGGCGCGTTCAGGTAAACCGACTATGTTTCTGATTCATACGCAG
GAAGGCGGTGGTGGTGATGCCGCAGCTGAAAATTTGGCCAAATGGTTTCAAAATGGTAATGGAGTGTCCTATCAT
TATACCATTAGCCAGGCAAGCGACGGCGGCGTAACCGTAGTGGACTGTGTAGATACGGATCGTGCGGCGTGGAGC
GTCGGAAATGCTAATAGTATTAGTATTAATTTGTGTTTTGCTGGATCACGCGCGAGCTGGATGCGCGACCAATGG
ATGAAACAATCGAATGCTATTGATGTTGCCGCGTATCTGGCTGTTCAAGATGCCAAGAAATATGGTTTTACGCCC
TTGGTCGTACCGCCGCCGTACACCAACGGCCGTCCGGGGATTAGCGATCATCGCTGGGTTACTGATGTATTTAAA
TGGGGTACCCATACGGATGTTGGTGATTGGTTTCCTTGGGATTATTTTGCGGAGCGCGTTAATCATTGGGCAAAT
GGCGGTAAAACGGAACCGGAGCCACCTAAAGTTAAGCGTTTTCCTGATGATTGGAGCGATCGTGAGATTCTGGTT
GAAATTCTTCGCCAGTTACGTGGGTATAATTTGACCGGGTGGCCACAATTGGGTGGCAAGACACTGGTAGATGCT
GTGGCGGAGTTGCTGGGACATGGCTCATAATAGTGATAATAG
Sequence Id4
  (SEQ ID NO: 150)
MHHHHHHSSGVDLGTENLYFQSMKRKKRKKRKGALNGEFYVPAGTIITANGTGVPDGAGPGFGADLARELERVAE
GMWIWKWINYPAAVMPMRPSINTLRLALKEMIRTTPGKIVLSAYSQSAVAFAYVWRDDILNPDGELHDRLDDIVA
VILYGNPVRAPGIAYGNELGGQSAPGKLNGHVTGGIAGPDCLRPEECVHPVTGRRIVLDFANAGDLYAAAPVGAE
PWVKETEVGHNETLIYEAVMDENGRDILAFAKEIAQILTMPLSQVVPLVQAIINGLSFLVQGPKAPHWTYTIGPA
VDYLVRTGNELRKNAGPAVPPPGSTTKDQVAQITIAEAKARGYTRSECLAIMSTFYQESGWNDTIWDPTHTTYGI
AQQDGSYPHRFDGAAAQIKGFFDKLDVWRAKPGASTDIWLNICWMQQAPNWPSADYWYANGRRAYLTEIKSRIAT
VTPYLDKYWPADGGTAVPDEPRPDFNEFPIWSNNNSARSGKPTMFLIHTQEGGGGDAAAENLAKWFQNGNGVSYH
YTISQASDGGVTVVDCVDTDRAAWSVGNANSISINLCFAGSRASWMRDQWMKQSNAIDVAAYLAVODAKKYGFTP
LVVPPPYTNGRPGISDHRWVTDVFKWGTHTDVGDWFPWDYFAERVNHWANGGKTEPEPPKVKRFPDDWSDREILV
EILRQLRGYNLTGWPQLGGKTLVDAVAELLGHGS
Sequence Id5 (1B11)
  (SEQ ID NO: 151)
ATGCACCATCATCATCATCATTCTTCTGGTGTAGATCTGGGTACCGAGAACCTGTACTTCCAATCCATGAAACGC
AAGAAACGTAAGAAACGCAAAGGTGCACGTATCGACGGCCAATATGTTGGATTAGGACCAGGGGATCGTTCGGAC
GAGATCCGCAAGATCAAAGCGTTCATGCGTCGCAAGTTCTCCTACGCAGCGACGCTGGCCGACACTGAGTTCTAC
GACGAGGCCATGACCGCTGTAGTCGCCGAGATGCAATCCCGTTACAACACTGCTGGTCAGCTCCGCGACGGGTTG
TACATCCCCGGGATTATCAACGCTGAAACCAAGTACGTTATGGGATATTTGTCACGTCCAGTAATCGACACACGT
CCAGTCCTGTTTACCGTATGCGGAACCGGCGTGCCTTGGTGGGTCGGCCCAGACGCCGACACCGCTCGCGCTGTT
GAGGACCAATATTTATGGCAACCGATTGGTTATCCGGCCGCGCCCTTCCCAATGGGTCGTTCCATCACCGCGGGG
ATCACTGAGGCACACAACCAGGCGAACCGCTGGCGCGAACGTATCGAAACTCACGGCACCGCCCTCGCAGGTTAT
TCTCAAGGCGCAGTCGTTCTTTCTGAGTTATGGATGAACCACATCGCACCGGAAGACGGCTCGCTTCGCTGGATG
AAACCACATGTACGTAAAGCAGTGACGTGGGGCAACCCGAACCGCGAATTAGGCCACGTGTGGGCCGATCACGGC
GGCTCTCCTATGGCGCCAAGTAACACTCAGGGCGTGTCGTCAAACGGAATGCGCAACACGCCTGACTGGTGGCGC
GACTACGCCCACCAAGGCGACCTGTACGCTTGCACCGAACCGGGAGACACCCAAGAGGTTCGCAACGCGATCTGG
CAGATCGTCCGCGACCTGGACCTGTTCACCGGACCAGATTCACTCCTTGCCCAAGTGATCGAACTGGCCCAAGCC
CCTCTCCCTGAGACGATCGCGATCACTCGCGCGATCCTGGACGCGGGTATGTTCTTCGCGAAACGCACAGGCCCA
CACGTAGACTACAACCCGCAGCCAGCGATCGACTATCTCCGCACGGCAGGTGCGGGTGCGGGTGCGGGTGGAAGC
ACAGAGCGTGTCCTGCCGTATGATCGTAATATTGTACCACAAGAAACGGGTTGGTGGTGCGGTCCGGCAGCGACG
CAAGTTGTTCTGAATAGTCGTGGAATTATTGTACCGGAAGCGACCCTGGCAAATGAAATTGAACAAATTGAAAAT
CCAGGTCGCGGTGACGATCGTGATGGAACGGATTATGTGGGGCTTATTGAACAAGTGCTTGATCGCCGTGTGCCC
GAAGCGAAATATACGAGCGTGTATATGCCTAATGATCCGCCCACCGCAGCCCAACGCGAAGCTTTATGGCAAAAT
ATTGTGCGCTCCATTAATGCGGGCTGGGGTGTCATTATGAATTGGGTGGCGCCGCCATCCAATAAACCACGCGGA
GTCAAAGGTAGCCCCAATCCTAAATATTCAGGTGGTACAACTTATCATTATGTGGCCTGTATGGGTTATGATGAT
ACTCCGGGCGCCCGCGCCTTGTGGATTGCGGATTCTGGGTTTCAACCCTTTAATTATTGGATTTCGTTTGATCAA
GCAGCAACGCTCATTCCACCTAAAGGGTATGCGTATGCCGCGACGACGGCTACAGCTCCTGCGCCGGCTCCAGCC
CCTGTTGATGCGGCACCCATTTTAGCACGCGCAGCTGGGATTAGCGAAGCTAAAGCACGTGAAATCTTACCCACC
TTTCGTGATGGTTTGCGCTTAGCTGAATGTAATAATGTTCCGCGCATTGCAATGTTTATTGCACAAACAGGCCAC
GAAAGCGATAATTTTAATGCCACTCAAGAATATGATCATGGCCGTAATTATGGCGATCCAAATGAAGCAACCGAT
CGTTGGAAATATAAAGGGCGTACGTGGATCCAAATTACATGGCGTGGTAATTATGAGCGTTTCTCACGTTGGTGT
TTTGATCGTAAATTGGTGCCTACCCCAACATATTTTGTAGATAATCCACGCGCACTCGCGGATGTTCGTTGGGCA
GGAATTGGCGCGGCGTGGTATTGGACGGTAGAACGTCCTTCCATTAATCGCCTGTGTGATGAGCGTAATTTAACG
GAAGTTACTCGTCTGATTAATGGTGGCACATCGTGGGAAGCCCCAACCTGGATGAAACATCGTAAAGAACGTTAT
GATCGTGCGTTAGCTGTGGGTAATGATCTTTTAAAGCTGTTAGATGGCGAAGAAGAAGGGTTTCTGTCGGCGCTT
ACGCCGAAAGAGCAACGTGAACTTTATGATGAAATTATGAAACGTGGGCCTAGCCGTAGCTTTCTTGCGGATGAT
GGTCGTCAAATCGAAACGCTGCTCGGTTTTATTTATAATATTGATGGAAATGCGTGGAATATTGTGAATATTCTT
GGTTGTTTGATTGGTGTACCAGAGTGTGTCGATGATATTCGTGACGTTGCCGAAAATGGAGTACGTAAAGGTTCA
TATGCAGAAAGTAATCCCTGGCTCGCGCAATTTGGTCAAGAATTTTGTAAACGTTTGCTTCCCCTTGCTGGTAAA
CTGTCTGAACTCTTGAATGCACAAGAATTTAAGGTTACACATAATCGCATTATTGAACAAGGCTCATAATAGTGA
TAATAG
Sequence Id6
  (SEQ ID NO: 152)
MHHHHHHSSGVDLGTENLYFQSMKRKKRKKRKGARIDGQYVGLGPGDRSDEIRKIKAFMRRKFSYAATLADTEFY
DEAMTAVVAEMQSRYNTAGQLRDGLYIPGIINAETKYVMGYLSRPVIDTRPVLFTVCGTGVPWWVGPDADTARAV
EDQYLWQPIGYPAAPFPMGRSITAGITEAHNQANRWRERIETHGTALAGYSQGAVVLSELWMNHIAPEDGSLRWM
KPHVRKAVTWGNPNRELGHVWADHGGSPMAPSNTQGVSSNGMRNTPDWWRDYAHQGDLYACTEPGDTQEVRNAIW
QIVRDLDLFTGPDSLLAQVIELAQAPLPETIAITRAILDAGMFFAKRTGPHVDYNPQPAIDYLRTAGAGAGAGGS
TERVLPYDRNIVPQETGWWCGPAATQVVLNSRGIIVPEATLANEIEQIENPGRGDDRDGTDYVGLIEQVLDRRVP
EAKYTSVYMPNDPPTAAQREALWQNIVRSINAGWGVIMNWVAPPSNKPRGVKGSPNPKYSGGTTYHYVACMGYDD
TPGARALWIADSGFQPFNYWISFDQAATLIPPKGYAYAATTATAPAPAPAPVDAAPILARAAGISEAKAREILPT
FRDGLRLAECNNVPRIAMFIAQTGHESDNFNATQEYDHGRNYGDPNEATDRWKYKGRTWIQITWRGNYERFSRWC
FDRKLVPTPTYFVDNPRALADVRWAGIGAAWYWTVERPSINRLCDERNLTEVTRLINGGTSWEAPTWMKHRKERY
DRALAVGNDLLKLLDGEEEGFLSALTPKEQRELYDEIMKRGPSRSFLADDGRQIETLLGFIYNIDGNAWNIVNIL
GCLIGVPECVDDIRDVAENGVRKGSYAESNPWLAQFGQEFCKRLLPLAGKLSELLNAQEFKVTHNRIIEQGS
Sequence Id7 (1C9)
  (SEQ ID NO: 153)
ATGCACCATCATCATCATCATTCTTCTGGTGTAGATCTGGGTACCGAGAACCTGTACTTCCAATCCATGTTCTTC
CATCATATTTTCCGCGGCAAAGTCCATGTGGGCAAGACCATTCATCGCTTAGTAACGGGTGGTGCTTCTAAGCCT
GTGCTGTTGACTGCTAGTGGGACGGGTGCGGATATGTGGTCTGGATATCCGGCGGATCTTGCGCGTCGCGTCGAG
GACCTTTACTACTTTCAGCCAATCTACTATGGTCCAGGTGGGATTCCGGCGACGGTACCAATGGGGGCGAGCGCA
CAGTCTGGCGTAGATATGGGAGTACGTCTGGTACTGGAGTGCGAGGCTCGTCCGTCGCGCGACGTCCCTGATGGG
TATGCCTTGTGTGGCTACTCTCAAGGGGGCATGGTTGTATCCGCGTTGTTGGACGAGTTTCGCACGGGCCGCCTT
CGCCACTTGAATGGCAAATTGATGGCGGGCAGCACCTTTGGAAACCCGTGGCGCGAGCTTGATTCTAACGGCGGG
CGCGGCATCAGCGACCGTCGCATTGTGGGGACCCCCGACTTCTGGGTGGATGAGTTCGACCCAGGAGACATTTAT
GCAAACGTGCCTAACAATGATGTCGGGGATGATATGACTGCCATTTTTAAGCTGGTACAGCTGCGCTCACTGGCT
GACCTGATCGGTATTGGATCGATTATGGATCGCGTAATGGACGTTCTTCGCGGCCCATTGAGCGGGATGCGCTCC
GTAGTTGAGGCGATTGTTCGTGGCTTATTATTTTTCGGCCGTAAGCCGCCAACAGCGCCCCATACAGAGTACCAT
TTGCGCGAGGTCCGTCCGGGAGTGACGTATTTTGAACATGCAGTCGCCCACATGCGCGCTATGGCCGCCGCAGGT
ATCCCGCAGGGTCGTTCTCACCCGGTTCAGCCGTACCCGGGTGCGTTCGGAAGCCCTGTCTATGCGATTCAGTCT
GGAACTGTTATTTACGCCGGTGCCGCATCGGGATATGGTGGTCCTGACCCTGCAGGCTGGCTTGTTATTGATTCT
GATGATGCCGAGGGTGGCGGCTGTTTAGAGTATGGTCATATTGTCCGTGAGGTAAATCGTGGTGATCATGTGACG
GCGGGTCAACGTATTGGACATATTAATCCAAATAATCGTACGAATGGTGGAGTGGCCCCGCATCTTCATGTAACT
GACTGGCCACATGCGTATGGGCAAGGCAGCCGTCAAAATGTGATGGCACGCTTGAGTGGAGCCCGCGAGCCAGAA
GCCCAAGCGGCCCCAACCCCCACAGAGCAAGGCACTGTTGTTGGTGACCCGGTTTGGTTGGCGGATGTAGTGCGC
CCAACTGTAGCTAAATTTGCGGAGTATCCTGGCTGGCGTAATCGTGGTCACGGTGATTTTAAGGATATCCGTGGA
GTTATGGTTCATCATACAGGTGGGCCCGCCTCTGCAGCCTCAATTGCGAATGGCCGCCCAGACCTTGCCGGCCCT
CTTGCACAGCTGCATATTTCGCGTGATGGGACAGTTACTGTCGTAGCTGTAGGAGTGGCTTGGCATGCGGGTGCA
GGCAGCTATCCATGGTTACCGACCAACATGGGTAATTGGCACTTGATTGGTATTGAATGTGAGTGGCCATATGGT
GAGCCGGGAATTAATGAGCGTAATGCCTATACAGTACGCTGGCGTGATCCAGAGATTATTGCGCTGCGTAATACA
TGTGCGGCAATTTTACTGCGCCTTGGCCTCGGGGTAGATCGCCTGATTGGCCATAAGGATTATGCCGGCCGTGCT
CAAGGCAAGTGGGACCCGGGAAATATGAGCATGGACTGGCTGCGTGGCGAGGTGCGTAAAGATATGGATGGCTTT
GTATTTCCCGGTGAGGATATTGTAAATGTGACCCCGCCACCAGTCCCGGCCCCTGTCCCTGCCCCGCCAGCGACT
GCGAATGTTCTTCTCCATCGTGGTATGAGTGGACCTAATGTTGTCAAATTACAGACAACTCTCCGTCGTTGGTAT
TCAAAACTGGCGGTAGACGGTGATTTTGGCCCCCATACAGAGGCATGTGTACGCGATCGCCAACGCATTTATGGG
TTGGACGTGGATGGTATTGTCGGACCATTAACTGCGGCCAAAATCGGCCTGGTTTTGGGCTCATAATAGTGATAA
TAG
Sequence Id8
  (SEQ ID NO: 154)
MHHHHHHSSGVDLGTENLYFQSMFFHHIFRGKVHVGKTIHRLVTGGASKPVLLTASGTGADMWSGYPADLARRVE
DLYYFQPIYYGPGGIPATVPMGASAQSGVDMGVRLVLECEARPSRDVPDGYALCGYSQGGMVVSALLDEFRTGRL
RHLNGKLMAGSTFGNPWRELDSNGGRGISDRRIVGTPDFWVDEFDPGDIYANVPNNDVGDDMTAIFKLVQLRSLA
DLIGIGSIMDRVMDVLRGPLSGMRSVVEAIVRGLLFFGRKPPTAPHTEYHLREVRPGVTYFEHAVAHMRAMAAAG
IPQGRSHPVQPYPGAFGSPVYAIQSGTVIYAGAASGYGGPDPAGWLVIDSDDAEGGGCLEYGHIVREVNRGDHVT
AGQRIGHINPNNRTNGGVAPHLHVTDWPHAYGQGSRQNVMARLSGAREPEAQAAPTPTEQGTVVGDPVWLADVVR
PTVAKFAEYPGWRNRGHGDFKDIRGVMVHHTGGPASAASIANGRPDLAGPLAQLHISRDGTVTVVAVGVAWHAGA
GSYPWLPTNMGNWHLIGIECEWPYGEPGINERNAYTVRWRDPEIIALRNTCAAILLRLGLGVDRLIGHKDYAGRA
QGKWDPGNMSMDWLRGEVRKDMDGFVFPGEDIVNVTPPPVPAPVPAPPATANVLLHRGMSGPNVVKLOTTLRRWY
SKLAVDGDFGPHTEACVRDRQRIYGLDVDGIVGPLTAAKIGLVLGS
Sequence Id9 (1E10)
  (SEQ ID NO: 155)
ATGCACCATCATCATCATCATTCTTCTGGTGTAGATCTGGGTACCGAGAACCTGTACTTCCAATCCATGTGGCGC
TCCCTGGGTCGTACGCTGTTACGTCTGTCCCATGCTCTGAAACCGCTGGCGCGCCGTTCCGGGTGGGGTGCTAGT
AAACCGTGGTTATTTACTGTGCATGGAACCGGACAACCCGATCCTCTTGGTCCCGGGCTCCCGGCAGACACAGCT
CGTGATGTTTTGGATATTTATCGTTGGCAACCAATTGGTAATTATCCAGCCGCCGCATTTCCCATGTGGCCCAGC
GTGGAGAAAGGGGTTGCCGAATTAATTCTCCAAATTGAACTTAAATTGGATGCGGACCCATATGCCGATTTTGCC
TTGGCGGGTTATTCTCAAGGTGCGATTGTTGTAGGACAAGTTCTGAAACATCATATTATTAATCCTCGCGGACGT
TTGCATCGTTTTCTTCATCGTCTGCGCAAAGTAATTTTCTGGGGCAACCCCATGCGTCAGAAAGGTTTCGCGCAT
ACTGATGAATGGATCCATCAAGTGGCCGCAAGTGATACTATGGGTATTCTGGAAGATCGTTTGGAAAATCTTGAA
CAGTATGGTTTCGAAGTTCGTGATTATGCGCATGATGGTGATATGTACGCGAGTATTAAAGAAGATGATATGCAT
GAATATGAAGTTGCGATCGGTCGTATTGTTATGTCTGCGCGTCGCTTTATTGGCGGAAAAGATTCAGTAATTGCG
CAACTGATTGAACTGGGCCAACGCCCAATTTGGGAAGGCATTGCAATGGCTCGCGCTATTATTGATGCGCTTACC
TTCTTCGCGAAATCTACACAGGGGCCTTCGTGGCCTCACCTTTATAATCGTTTTCCAGCCGTAGAATTTCTGCGT
CGCATTGCAGGTGCGGGTGCGGGTGCGGGTGGAAGCTCATTTACGCGCTTTCTCCAAGACGATCCCTTGCTGACT
CGTGAACAGGTAATGGCAGAACTCATCCGCGTTGCTGATGAATTGAATATGCCTGATAAACGTGGAGCATGTGTG
ATCGCAGGAATGACTATCTCACAGGAAGTTGGTGTGAAAGATAATGATCCCCCATTTGAACGCCGTTTCTGGTGT
CCAGCAAATCGTGCGGATCCAGAGTCTTTTAATTATCCACATGATAGTGAGTCCAATGATGGACGTTCAGTGGGG
TATTTTCAACAACAAAAGGGCCCAAATGGTGAACTTTGGTGGGGCACTACCGCGAGTGAAATGAATTTACATAGT
GCAGCCACCCAATTCATGACTCGTTTAAAAGCCGCGGGTTATAATGCCTCTAATGCGCAAGCCGCAAATGATTCT
GCTCAAGCTATTCAACGTTCAGGTGTTCCACAAGCCTATAAACAGTGGTGGGATGATATCAATCGTTTATATGAT
AAAGTAAAAGGTAGCGGCGGCGGACCCGCACCGGCTCCCAAACCACCTCAAAGCGGTCCTTGGACTGGTGATCCT
GTTTGGTTAGCAGATGTATTACGTGCAGAAGGCTTGAATGTTGTTGAACTCCCTGGTTGGTTAGATCGTGGTCAT
GGAGATATGGGTCGTCTGTGGGGCGTCGTATGTCACCATACTGGTAGTGACAATACTCCAAGCTCTGAAATCGCA
TTCCATCCCAGCCTGGGTCTTTGTTCCCAAATCCATCTTGCCCGTAATGGTACGGTTACACTTTGTGGCGTTGGT
ATTGCGTGGCACGCAGGAGTGGGATCGTACCCGGGGTTGCCGGAAGATAATGCAAATGCCGTGACAATTGGTATT
GAAGCACAAAATTCGGGTACATACGATGGTGCTCCTCATCGCACCAACTGGCCGGATGCACAGTATGATGCGTAC
GTCAAATGTTGTGCGGCTATTTGTCGTCGTCTGGGAGTCCGTGCAGACCATGTAATTTCGCATAAAGAGTGGGCG
GGCCGTAAACAGGGTAAGTGGGACCCGGGAGCGATTGATATGAATATTTTCCGTGCGGATGTTCAACGTCGTATT
GATGCGCACCAGCCGAATGGTGAAGATGACTTTATGGCGGCTCTGAGCGCGGATGAACAACGTGAAGTTTTGAAT
CTTTTGCGTGTTTTAGCGGATCGCCGCTTTGTGAGTCGTTCACCTTTTCGTCATCTCGGTGAAGGTCCATCCGAG
ACGGTAGCGGGCTTTGGTTTAAATACGGATGGACTGAACCATGCTCAATATACCATCGAACTCGCACGTCTCGGG
GATCCTACCCATTTAGCGTTACTCCGTGAAGTAGCTAGTGCGGAAGGGGATTCTCGTTACCCGGATCGCCAATAT
GATGCGAAATTGGCGAAACGTGTCTTGGCTGAGATTGAAGGTGCGGCGACCGCGCCTGCGAAACCTTCTACTCCT
TCAGCGCCAACGGAACCAGCACCGGAAGCACCTACTCCACCGGTGAAAGCGGCTTGTGCACTCAGCGCCGCGGGC
TGTGTAGTAGCGGGGAGCACTTCAGGTGGTGGTTGTGCGTTATCGACAGATGGTACGGGGAAATGTGTGGTCACT
GCGGCCACTGATGGTGGCGCGGCAGGCTCATAATAGTGATAATAG
Sequence Id10
  (SEQ ID NO: 156)
MHHHHHHSSGVDLGTENLYFQSMWRSLGRTLLRLSHALKPLARRSGWGASKPWLFTVHGTGQPDPLGPGLPADTA
RDVLDIYRWQPIGNYPAAAFPMWPSVEKGVAELILQIELKLDADPYADFALAGYSQGAIVVGQVLKHHIINPRGR
LHRFLHRLRKVIFWGNPMRQKGFAHTDEWIHQVAASDTMGILEDRLENLEQYGFEVRDYAHDGDMYASIKEDDMH
EYEVAIGRIVMSARRFIGGKDSVIAQLIELGQRPIWEGIAMARAIIDALTFFAKSTQGPSWPHLYNRFPAVEFLR
RIAGAGAGAGGSSFTRFLQDDPLLTREQVMAELIRVADELNMPDKRGACVIAGMTISQEVGVKDNDPPFERREWC
PANRADPESFNYPHDSESNDGRSVGYFQQQKGPNGELWWGTTASEMNLHSAATQFMTRLKAAGYNASNAQAANDS
AQAIQRSGVPQAYKQWWDDINRLYDKVKGSGGGPAPAPKPPQSGPWTGDPVWLADVLRAEGLNVVELPGWLDRGH
GDMGRLWGVVCHHTGSDNTPSSEIAFHPSLGLCSQIHLARNGTVTLCGVGIAWHAGVGSYPGLPEDNANAVTIGI
EAQNSGTYDGAPHRTNWPDAQYDAYVKCCAAICRRLGVRADHVISHKEWAGRKQGKWDPGAIDMNIFRADVQRRI
DAHQPNGEDDFMAALSADEQREVLNLLRVLADRRFVSRSPFRHLGEGPSETVAGFGLNTDGLNHAQYTIELARLG
DPTHLALLREVASAEGDSRYPDRQYDAKLAKRVLAEIEGAATAPAKPSTPSAPTEPAPEAPTPPVKAACALSAAG
CVVAGSTSGGGCALSTDGTGKCVVTAATDGGAAGS
Sequence Id11 (1G8)
  (SEQ ID NO: 157)
ATGCACCATCATCATCATCATTCTTCTGGTGTAGATCTGGGTACCGAGAACCTGTACTTCCAATCCATGGGCATT
GGTAAGTTCCTGAAAAAAGCGAAGAAATTTGGTGCTAGTAAACCGTGGTTATTTACTGTGCATGGAACCGGACAA
CCCGATCCTCTTGGTCCCGGGCTCCCGGCAGACACAGCTCGTGATGTTTTGGATATTTATCGTTGGCAACCAATT
GGTAATTATCCAGCCGCCGCATTTCCCATGTGGCCCAGCGTGGAGAAAGGGGTTGCCGAATTAATTCTCCAAATT
GAACTTAAATTGGATGCGGACCCATATGCCGATTTTGCCTTGGCGGGTTATTCTCAAGGTGCGATTGTTGTAGGA
CAAGTTCTGAAACATCATATTATTAATCCTCGCGGACGTTTGCATCGTTTTCTTCATCGTCTGCGCAAAGTAATT
TTCTGGGGCAACCCCATGCGTCAGAAAGGTTTCGCGCATACTGATGAATGGATCCATCAAGTGGCCGCAAGTGAT
ACTATGGGTATTCTGGAAGATCGTTTGGAAAATCTTGAACAGTATGGTTTCGAAGTTCGTGATTATGCGCATGAT
GGTGATATGTACGCGAGTATTAAAGAAGATGATATGCATGAATATGAAGTTGCGATCGGTCGTATTGTTATGTCT
GCGCGTCGCTTTATTGGCGGAAAAGATTCAGTAATTGCGCAACTGATTGAACTGGGCCAACGCCCAATTTGGGAA
GGCATTGCAATGGCTCGCGCTATTATTGATGCGCTTACCTTCTTCGCGAAATCTACACAGGGGCCTTCGTGGCCT
CACCTTTATAATCGTTTTCCAGCCGTAGAATTTCTGCGTCGCATTGCAGGTTTAAGCCGCTTCTTCCATGCCGAG
TTGGGAAGCCCTGTCTATGCGATTCAGTCTGGAACTGTTATTTACGCCGGTGCCGCATCGGGATATGGTGGTCCT
GACCCTGCAGGCTGGCTTGTTATTGATTCTGATGATGCCGAGGGTGGCGGCTGTTTAGAGTATGGTCATATTGTC
CGTGAGGTAAATCGTGGTGATCATGTGACGGCGGGTCAACGTATTGGACATATTAATCCAAATAATCGTACGAAT
GGTGGAGTGGCCCCGCATCTTCATGTAACTGACTGGCCACATGCGTATGGGCAAGGCAGCCGTCAAAATGTGATG
GCACGCTTGAGTGGAGCCCGCGAGCCAGAAGCCCAAGCGGCCCCAACCCCCACAGAGCAAGGCACTGTTGTTGGT
GACCCGGTTTGGTTGGCGGATGTAGTGCGCCCAACTGTAGCTAAATTTGCGGAGTATCCTGGCTGGCGTAATCGT
GGTCACGGTGATTTTAAGGATATCCGTGGAGTTATGGTTCATCATACAGGTGGGCCCGCCTCTGCAGCCTCAATT
GCGAATGGCCGCCCAGACCTTGCCGGCCCTCTTGCACAGCTGCATATTTCGCGTGATGGGACAGTTACTGTCGTA
GCTGTAGGAGTGGCTTGGCATGCGGGTGCAGGCAGCTATCCATGGTTACCGACCAACATGGGTAATTGGCACTTG
ATTGGTATTGAATGTGAGTGGCCATATGGTGAGCCGGGAATTAATGAGCGTAATGCCTATACAGTACGCTGGCGT
GATCCAGAGATTATTGCGCTGCGTAATACATGTGCGGCAATTTTACTGCGCCTTGGCCTCGGGGTAGATCGCCTG
ATTGGCCATAAGGATTATGCCGGCCGTGCTCAAGGCAAGTGGGACCCGGGAAATATGAGCATGGACTGGCTGCGT
GGCGAGGTGCGTAAAGATATGGATGGCTTTGTATTTCCCGGTGAGGATATTGTAAATGTGACCCCGCCACCAGTC
CCGGCCCCTGTCCCTGCCCCGCCAGCGACTGCGAATGTTCTTCTCCATCGTGGTATGAGTGGACCTAATGTTGTC
AAATTACAGACAACTCTCCGTCGTTGGTATTCAAAACTGGCGGTAGACGGTGATTTTGGCCCCCATACAGAGGCA
TGTGTACGCGATCGCCAACGCATTTATGGGTTGGACGTGGATGGTATTGTCGGACCATTAACTGCGGCCAAAATC
GGCCTGGTTTTGGGCTCATAATAGTGATAATAG
Sequence Id12
  (SEQ ID NO: 158)
MHHHHHHSSGVDLGTENLYFQSMGIGKFLKKAKKFGASKPWLFTVHGTGQPDPLGPGLPADTARDVLDIYRWQPI
GNYPAAAFPMWPSVEKGVAELILQIELKLDADPYADFALAGYSQGAIVVGQVLKHHIINPRGRLHRFLHRLRKVI
FWGNPMRQKGFAHTDEWIHQVAASDTMGILEDRLENLEQYGFEVRDYAHDGDMYASIKEDDMHEYEVAIGRIVMS
ARRFIGGKDSVIAQLIELGQRPIWEGIAMARAIIDALTFFAKSTQGPSWPHLYNRFPAVEFLRRIAGLSRFFHAE
LGSPVYAIQSGTVIYAGAASGYGGPDPAGWLVIDSDDAEGGGCLEYGHIVREVNRGDHVTAGQRIGHINPNNRTN
GGVAPHLHVTDWPHAYGQGSRQNVMARLSGAREPEAQAAPTPTEQGTVVGDPVWLADVVRPTVAKFAEYPGWRNR
GHGDFKDIRGVMVHHTGGPASAASIANGRPDLAGPLAQLHISRDGTVTVVAVGVAWHAGAGSYPWLPTNMGNWHL
IGIECEWPYGEPGINERNAYTVRWRDPEIIALRNTCAAILLRLGLGVDRLIGHKDYAGRAQGKWDPGNMSMDWLR
GEVRKDMDGFVFPGEDIVNVTPPPVPAPVPAPPATANVLLHRGMSGPNVVKLQTTLRRWYSKLAVDGDFGPHTEA
CVRDRQRIYGLDVDGIVGPLTAAKIGLVLGS
Sequence Id13 (1H4)
  (SEQ ID NO: 159)
ATGCACCATCATCATCATCATTCTTCTGGTGTAGATCTGGGTACCGAGAACCTGTACTTCCAATCCATGTTCTTC
CATCATATTTTCCGCGGCAAAGTCCATGTGGGCAAGACCATTCATCGCTTAGTAACGGGTGGTGCTTCTAAGCCT
GTGCTGTTGACTGCTAGTGGGACGGGTGCGGATATGTGGTCTGGATATCCGGCGGATCTTGCGCGTCGCGTCGAG
GACCTTTACTACTTTCAGCCAATCTACTATGGTCCAGGTGGGATTCCGGCGACGGTACCAATGGGGGCGAGCGCA
CAGTCTGGCGTAGATATGGGAGTACGTCTGGTACTGGAGTGCGAGGCTCGTCCGTCGCGCGACGTCCCTGATGGG
TATGCCTTGTGTGGCTACTCTCAAGGGGGCATGGTTGTATCCGCGTTGTTGGACGAGTTTCGCACGGGCCGCCTT
CGCCACTTGAATGGCAAATTGATGGCGGGCAGCACCTTTGGAAACCCGTGGCGCGAGCTTGATTCTAACGGCGGG
CGCGGCATCAGCGACCGTCGCATTGTGGGGACCCCCGACTTCTGGGTGGATGAGTTCGACCCAGGAGACATTTAT
GCAAACGTGCCTAACAATGATGTCGGGGATGATATGACTGCCATTTTTAAGCTGGTACAGCTGCGCTCACTGGCT
GACCTGATCGGTATTGGATCGATTATGGATCGCGTAATGGACGTTCTTCGCGGCCCATTGAGCGGGATGCGCTCC
GTAGTTGAGGCGATTGTTCGTGGCTTATTATTTTTCGGCCGTAAGCCGCCAACAGCGCCCCATACAGAGTACCAT
TTGCGCGAGGTCCGTCCGGGAGTGACGTATTTTGAACATGCAGTCGCCCACATGCGCGCTATGGCCGCCGCAGGT
CCAGCCGTTCCACCTCCGGGAAGCCCTGTCTATGCGATTCAGTCTGGAACTGTTATTTACGCCGGTGCCGCATCG
GGATATGGTGGTCCTGACCCTGCAGGCTGGCTTGTTATTGATTCTGATGATGCCGAGGGTGGCGGCTGTTTAGAG
TATGGTCATATTGTCCGTGAGGTAAATCGTGGTGATCATGTGACGGCGGGTCAACGTATTGGACATATTAATCCA
AATAATCGTACGAATGGTGGAGTGGCCCCGCATCTTCATGTAACTGACTGGCCACATGCGTATGGGCAAGGCAGC
CGTCAAAATGTGATGGCACGCTTGAGTGGAGCCCGCGAGCCAGAAGCCCAAGCGGCCCCAACCCCCACAGAGCAA
GGCACTGTTGTTGGTGACCCGGTTTGGTTGGCGGATGTAGTGCGCCCAACTGTAGCTAAATTTGCGGAGTATCCT
GGCTGGCGTAATCGTGGTCACGGTGATTTTAAGGATATCCGTGGAGTTATGGTTCATCATACAGGTGGGCCCGCC
TCTGCAGCCTCAATTGCGAATGGCCGCCCAGACCTTGCCGGCCCTCTTGCACAGCTGCATATTTCGCGTGATGGG
ACAGTTACTGTCGTAGCTGTAGGAGTGGCTTGGCATGCGGGTGCAGGCAGCTATCCATGGTTACCGACCAACATG
GGTAATTGGCACTTGATTGGTATTGAATGTGAGTGGCCATATGGTGAGCCGGGAATTAATGAGCGTAATGCCTAT
ACAGTACGCTGGCGTGATCCAGAGATTATTGCGCTGCGTAATACATGTGCGGCAATTTTACTGCGCCTTGGCCTC
GGGGTAGATCGCCTGATTGGCCATAAGGATTATGCCGGCCGTGCTCAAGGCAAGTGGGACCCGGGAAATATGAGC
ATGGACTGGCTGCGTGGCGAGGTGCGTAAAGATATGGATGGCTTTGTATTTCCCGGTGAGGATATTGTAAATGTG
ACCCCGCCACCAGTCCCGGCCCCTGTCCCTGCCCCGCCAGCGACTGCGAATGTTCTTCTCCATCGTGGTATGAGT
GGACCTAATGTTGTCAAATTACAGACAACTCTCCGTCGTTGGTATTCAAAACTGGCGGTAGACGGTGATTTTGGC
CCCCATACAGAGGCATGTGTACGCGATCGCCAACGCATTTATGGGTTGGACGTGGATGGTATTGTCGGACCATTA
ACTGCGGCCAAAATCGGCCTGGTTTTGGGCTCATAATAGTGATAATAG
Sequence Id14
  (SEQ ID NO: 160)
MHHHHHHSSGVDLGTENLYFQSMFFHHIFRGKVHVGKTIHRLVTGGASKPVLLTASGTGADMWSGYPADLARRVE
DLYYFQPIYYGPGGIPATVPMGASAQSGVDMGVRLVLECEARPSRDVPDGYALCGYSQGGMVVSALLDEFRTGRL
RHLNGKLMAGSTFGNPWRELDSNGGRGISDRRIVGTPDFWVDEFDPGDIYANVPNNDVGDDMTAIFKLVQLRSLA
DLIGIGSIMDRVMDVLRGPLSGMRSVVEAIVRGLLFFGRKPPTAPHTEYHLREVRPGVTYFEHAVAHMRAMAAAG
PAVPPPGSPVYAIQSGTVIYAGAASGYGGPDPAGWLVIDSDDAEGGGCLEYGHIVREVNRGDHVTAGQRIGHINP
NNRTNGGVAPHLHVTDWPHAYGQGSRQNVMARLSGAREPEAQAAPTPTEQGTVVGDPVWLADVVRPTVAKFAEYP
GWRNRGHGDFKDIRGVMVHHTGGPASAASIANGRPDLAGPLAQLHISRDGTVTVVAVGVAWHAGAGSYPWLPTNM
GNWHLIGIECEWPYGEPGINERNAYTVRWRDPEIIALRNTCAAILLRLGLGVDRLIGHKDYAGRAQGKWDPGNMS
MDWLRGEVRKDMDGFVFPGEDIVNVTPPPVPAPVPAPPATANVLLHRGMSGPNVVKLQTTLRRWYSKLAVDGDFG
PHTEACVRDRQRIYGLDVDGIVGPLTAAKIGLVLGS
Sequence 4D10
  (SEQ ID NO: 292)
WRSLGRTLLRLSHALKPLARRSGWMSKPWLFTVHGTGQPDPLGPGLPADTARDVLDIYRWQPIGNYPAAAFPMWP
SVEKGVAELILQIELKLDADPYADFAMAGYSQGAIVVGQVLKHHILPPTGRLHRFLHRLKKVIFWGNPMRQKGFA
HSDEWIHPVAAPDTLGILEDRLENLEQYGFEVRDYAHDGDMYASIKEDDLHEYEVAIGRIVMKASGFIGGRDSVV
AQLIELGQRPITEGIALAGAIIDALTFFARSRMGDKWPHLYNRYPAVEFLRQIAGAGAGMSFTRFLQDDPLLTRE
QVMAELIRVADELNMPDKRGACVIAGMTISQEVGVKDNDPPFERRFWCPANRADPESFNYPHDSESNDGRSVGYF
QQQKGPNGELWWGTTASEMNLHSAATQFMTRLKAAGYNASNAQAANDSAQAIQRSGVPQAYKQWWDDINRLYDKV
KGSGGGPAPAPKPPQSGPWTGDPVWLADVLRAEGLNVVELPGWLDRGHGDMGRLWGVVCHHTGSDNTPSSEIAFH
PSLGLCSQIHLARNGTVTLCGVGIAWHAGVGSYPGLPEDNANAVTIGIEAQNSGTYDGAPHRTNWPDAQYDAYVK
CCAAICRRLGVRADHVISHKEWAGRKQGKWDPGAIDMNIFRADVQRRIDAHQPNGEDDFMAALSADEQREVLNLL
RVLADRRFVSRSPFRHLGEGPSETVAGFGLNTDGLNHAQYTIELARLGDPTHLALLREVASAEGDSRYPDRQYDA
KLAKRVLAEIEGAATAPAKPSTPSAPTEPAPEAPTPPVKAACALSAAGCVVAGSTSGGGCALSTDGTGKCVVTAA
TDGGAA
Sequence 4D10 in reverse
  (SEQ ID NO: 312)
MSFTRFLQDDPLLTREQVMAELIRVADELNMPDKRGACVIAGMTISQEVGVKDNDPPFERRFWCPANRADPESEN
YPHDSESNDGRSVGYFQQQKGPNGELWWGTTASEMNLHSAATQFMTRLKAAGYNASNAQAANDSAQAIQRSGVPQ
AYKQWWDDINRLYDKVKGSGGGPAPAPKPPQSGPWTGDPVWLADVLRAEGLNVVELPGWLDRGHGDMGRLWGVVC
HHTGSDNTPSSEIAFHPSLGLCSQIHLARNGTVTLCGVGIAWHAGVGSYPGLPEDNANAVTIGIEAQNSGTYDGA
PHRTNWPDAQYDAYVKCCAAICRRLGVRADHVISHKEWAGRKQGKWDPGAIDMNIFRADVQRRIDAHQPNGEDDE
MAALSADEQREVLNLLRVLADRRFVSRSPFRHLGEGPSETVAGFGLNTDGLNHAQYTIELARLGDPTHLALLREV
ASAEGDSRYPDRQYDAKLAKRVLAEIEGAATAPAKPSTPSAPTEPAPEAPTPPVKAACALSAAGCVVAGSTSGGG
CALSTDGTGKCVVTAATDGGAAAGAGAGMSKPWLFTVHGTGQPDPLGPGLPADTARDVLDIYRWQPIGNYPAAAF
PMWPSVEKGVAELILQIELKLDADPYADFAMAGYSQGAIVVGQVLKHHILPPTGRLHRFLHRLKKVIFWGNPMRQ
KGFAHSDEWIHPVAAPDTLGILEDRLENLEQYGFEVRDYAHDGDMYASIKEDDLHEYEVAIGRIVMKASGFIGGR
DSVVAQLIELGQRPITEGIALAGAIIDALTFFARSRMGDKWPHLYNRYPAVEFLRQIWRSLGRTLLRLSHALKPL
ARRSGW
```

Fusion peptides of the invention may optionally comprise a purification fusion tag, e.g. MHHHHHHSSGVDLGTENLYFQS (SEQ ID NO: 339). The skilled person will appreciate that inclusion of such a purification fusion tag is optional, and the scope of the invention encompasses sequences including these fusion tags, or corresponding sequences with the fusion tags removed. Additionally, the skilled person will appreciate that any suitable fusion tag can be used as an alternative.

A peptide according to the present invention may be selected from any one or more of libraries 1-3 and 12-16. In some embodiments, the peptide according to the present invention may be selected from library 1, for example selected from the group consisting of SEQ ID NOS: 147-160, 201-265, 267-270, 272, 273, 275-308 and 311. In other embodiments, the peptide according to the present invention may be selected from library 12, for example selected from the group consisting of SEQ ID NOs: 161-200, 266, 271, 274, 309 and 310.

A peptide according to the present invention may be selected from the exemplary peptides in Table A.

TABLE A
Exemplary peptides of the invention. The following table includes in the white
row a combination of four SEQ ID NOs (e.g. the first cell is a combination
of SEQ ID NOs: 58, 12, 108 and 16), and the cell immediately underneath the
combination denotes the SEQ ID NO for the corresponding fusion protein (e.g.
the first cell as a fusion protein corresponds to SEQ ID NO: 161).

| | | | |
|---|---|---|---|
| 58 + 12 + 108 + 16 | 36 + 18 + 106 + 6 | 36 + 32 + 110 + 2 | 42 + 20 + 112 + 10 |
| SEQ ID NO: 161 | SEQ ID NO: 199 | SEQ ID NO: 237 | SEQ ID NO: 275 |
| 46 + 8 + 112 + 32 | 92 + 20 + 106 + 14 | 76 + 16 + 110 + 14 | 56 + 22 + 106 + 14 |
| SEQ ID NO: 162 | SEQ ID NO: 200 | SEQ ID NO: 238 | SEQ ID NO: 276 |
| 36 + 14 + 110 + 20 | 40 + 28 + 106 + 2 | 90 + 24 + 104 + 14 | 38 + 22 + 110 + 2 |
| SEQ ID NO: 163 | SEQ ID NO: 201 | SEQ ID NO: 239 | SEQ ID NO: 277 |
| 76 + 2 + 106 + 30 | 36 + 20 + 108 + 10 | 36 + 32 + 110 + 14 | 98 + 24 + 106 + 10 |
| SEQ ID NO: 164 | SEQ ID NO: 202 | SEQ ID NO: 240 | SEQ ID NO: 278 |
| 36 + 14 + 112 + 28 | 36 + 16 + 106 + 12 | 82 + 28 + 104 + 14 | 36 + 24 + 110 + 14 |
| SEQ ID NO: 165 | SEQ ID NO: 203 | SEQ ID NO: 241 | SEQ ID NO: 279 |
| 100 + 12 + 110 + 32 | 42 + 18 + 112 + 10 | 58 + 22 + 104 + 12 | 36 + 18 + 110 + 2 |
| SEQ ID NO: 166 | SEQ ID NO: 204 | SEQ ID NO: 242 | SEQ ID NO: 280 |
| 46 + 10 + 104 + 32 | 50 + 16 + 112 + 2 | 44 + 20 + 108 + 2 | 36 + 26 + 112 + 10 |
| SEQ ID NO: 167 | SEQ ID NO: 205 | SEQ ID NO: 243 | SEQ ID NO: 281 |
| 88 + 14 + 108 + 28 | 36 + 26 + 112 + 2 | 66 + 20 + 110 + 10 | 72 + 22 + 106 + 14 |
| SEQ ID NO: 168 | SEQ ID NO: 206 | SEQ ID NO: 244 | SEQ ID NO: 282 |
| 38 + 14 + 104 + 18 | 48 + 16 + 112 + 14 | 36 + 34 + 112 + 10 | 36 + 26 + 106 + 10 |
| SEQ ID NO: 169 | SEQ ID NO: 207 | SEQ ID NO: 245 | SEQ ID NO: 283 |
| 42 + 12 + 108 + 28 | 66 + 20 + 114 + 10 | 80 + 16 + 108 + 2 | 58 + 28 + 106 + 14 |
| SEQ ID NO: 170 | SEQ ID NO: 208 | SEQ ID NO: 246 | SEQ ID NO: 284 |
| 96 + 2 + 110 + 18 | 102 + 16 + 108 + 10 | 44 + 20 + 106 + 14 | 44 + 30 + 108 + 6 |
| SEQ ID NO: 171 | SEQ ID NO: 209 | SEQ ID NO: 247 | SEQ ID NO: 285 |
| 44 + 10 + 104 + 18 | 100 + 18 + 112 + 12 | 74 + 28 + 112 + 14 | 52 + 16 + 112 + 14 |
| SEQ ID NO: 172 | SEQ ID NO: 210 | SEQ ID NO: 248 | SEQ ID NO: 286 |
| 64 + 10 + 112 + 30 | 92 + 28 + 106 + 14 | 36 + 20 + 112 + 10 | 46 + 30 + 104 + 10 |
| SEQ ID NO: 173 | SEQ ID NO: 211 | SEQ ID NO: 249 | SEQ ID NO: 287 |
| 68 + 12 + 106 + 18 | 36 + 18 + 110 + 14 | 38 + 26 + 112 + 10 | 44 + 22 + 110 + 10 |
| SEQ ID NO: 174 | SEQ ID NO: 212 | SEQ ID NO: 250 | SEQ ID NO: 288 |
| 100 + 12 + 104 + 16 | 58 + 20 + 110 + 14 | 36 + 20 + 112 + 4 | 38 + 32 + 106 + 12 |
| SEQ ID NO: 175 | SEQ ID NO: 213 | SEQ ID NO: 251 | SEQ ID NO: 289 |
| 88 + 8 + 110 + 20 | 40 + 32 + 104 + 14 | 52 + 20 + 108 + 2 | 40 + 16 + 112 + 14 |
| SEQ ID NO: 176 | SEQ ID NO: 214 | SEQ ID NO: 252 | SEQ ID NO: 290 |
| 98 + 8 + 110 + 24 | 102 + 34 + 112 + 10 | 36 + 24 + 112 + 10 | 38 + 24 + 114 + 10 |
| SEQ ID NO: 177 | SEQ ID NO: 215 | SEQ ID NO: 253 | SEQ ID NO: 291 |
| 92 + 12 + 104 + 18 | 48 + 16 + 104 + 2 | 38 + 20 + 110 + 2 | 38 + 16 + 104 + 14 |
| SEQ ID NO: 178 | SEQ ID NO: 216 | SEQ ID NO: 254 | SEQ ID NO: 292 |
| 100 + 12 + 104 + 16 | 40 + 20 + 106 + 14 | 42 + 20 + 110 + 14 | 98 + 34 + 112 + 10 |
| SEQ ID NO: 179 | SEQ ID NO: 217 | SEQ ID NO: 255 | SEQ ID NO: 293 |
| 52 + 2 + 112 + 30 | 36 + 18 + 112 + 14 | 78 + 16 + 104 + 14 | 66 + 20 + 112 + 14 |
| SEQ ID NO: 180 | SEQ ID NO: 218 | SEQ ID NO: 256 | SEQ ID NO: 294 |
| 44 + 6 + 110 + 30 | 38 + 16 + 112 + 4 | 38 + 22 + 112 + 2 | 36 + 30 + 112 + 6 |
| SEQ ID NO: 181 | SEQ ID NO: 219 | SEQ ID NO: 257 | SEQ ID NO: 295 |
| 38 + 10 + 112 + 16 | 86 + 20 + 106 + 14 | 38 + 34 + 108 + 14 | 36 + 16 + 112 + 10 |
| SEQ ID NO: 182 | SEQ ID NO: 220 | SEQ ID NO: 258 | SEQ ID NO: 296 |
| 94 + 10 + 108 + 16 | 96 + 16 + 106 + 10 | 36 + 26 + 104 + 10 | 94 + 30 + 106 + 4 |
| SEQ ID NO: 183 | SEQ ID NO: 221 | SEQ ID NO: 259 | SEQ ID NO: 297 |
| 52 + 2 + 112 + 16 | 44 + 26 + 110 + 10 | 86 + 30 + 104 + 14 | 90 + 16 + 112 + 10 |
| SEQ ID NO: 184 | SEQ ID NO: 222 | SEQ ID NO: 260 | SEQ ID NO: 298 |
| 96 + 6 + 112 + 22 | 40 + 32 + 110 + 2 | 36 + 26 + 106 + 6 | 40 + 24 + 110 + 6 |
| SEQ ID NO: 185 | SEQ ID NO: 223 | SEQ ID NO: 261 | SEQ ID NO: 299 |
| 98 + 10 + 108 + 18 | 38 + 16 + 104 + 4 | 40 + 26 + 110 + 8 | 36 + 24 + 104 + 10 |
| SEQ ID NO: 186 | SEQ ID NO: 224 | SEQ ID NO: 262 | SEQ ID NO: 300 |
| 56 + 2 + 104 + 32 | 36 + 20 + 106 + 12 | 36 + 28 + 112 + 10 | 38 + 24 + 104 + 14 |
| SEQ ID NO: 187 | SEQ ID NO: 225 | SEQ ID NO: 263 | SEQ ID NO: 301 |
| 68 + 8 + 104 + 18 | 48 + 34 + 110 + 14 | 36 + 20 + 104 + 14 | 36 + 20 + 104 + 14 |
| SEQ ID NO: 188 | SEQ ID NO: 226 | SEQ ID NO: 264 | SEQ ID NO: 302 |
| 44 + 6 + 112 + 32 | 38 + 30 + 112 + 14 | 78 + 20 + 104 + 10 | 38 + 18 + 108 + 14 |
| SEQ ID NO: 189 | SEQ ID NO: 227 | SEQ ID NO: 265 | SEQ ID NO: 303 |
| 52 + 6 + 112 + 24 | 38 + 18 + 106 + 10 | 52 + 14 + 104 + 28 | 96 + 32 + 104 + 10 |
| SEQ ID NO: 190 | SEQ ID NO: 228 | SEQ ID NO: 266 | SEQ ID NO: 304 |
| 42 + 12 + 110 + 26 | 36 + 26 + 112 + 2 | 58 + 24 + 104 + 14 | 66 + 20 + 108 + 12 |
| SEQ ID NO: 191 | SEQ ID NO: 229 | SEQ ID NO: 267 | SEQ ID NO: 305 |
| 40 + 2 + 110 + 24 | 36 + 20 + 106 + 14 | 66 + 20 + 110 + 6 | 36 + 32 + 104 + 10 |
| SEQ ID NO: 192 | SEQ ID NO: 230 | SEQ ID NO: 268 | SEQ ID NO: 306 |
| 60 + 2 + 104 + 18 | 36 + 24 + 110 + 10 | 86 + 16 + 110 + 2 | 52 + 22 + 108 + 10 |
| SEQ ID NO: 193 | SEQ ID NO: 231 | SEQ ID NO: 269 | SEQ ID NO: 307 |
| 38 + 4 + 110 + 26 | 36 + 18 + 104 + 2 | 86 + 34 + 112 + 10 | 36 + 22 + 110 + 10 |
| SEQ ID NO: 194 | SEQ ID NO: 232 | SEQ ID NO: 270 | SEQ ID NO: 308 |
| 62 + 10 + 104 + 32 | 76 + 26 + 104 + 4 | 36 + 14 + 106 + 28 | 68 + 6 + 104 + 16 |
| SEQ ID NO: 195 | SEQ ID NO: 233 | SEQ ID NO: 271 | SEQ ID NO: 309 |
| 40 + 14 + 106 + 18 | 36 + 20 + 106 + 2 | 36 + 22 + 110 + 2 | 92 + 2 + 106 + 18 |
| SEQ ID NO: 196 | SEQ ID NO: 234 | SEQ ID NO: 272 | SEQ ID NO: 310 |
| 62 + 8 + 110 + 24 | 58 + 30 + 110 + 10 | 44 + 26 + 110 + 10 | 36 + 28 + 110 + 14 |
| SEQ ID NO: 197 | SEQ ID NO: 235 | SEQ ID NO: 273 | SEQ ID NO: 311 |
| 88 + 8 + 108 + 28 | 38 + 20 + 112 + 14 | 98 + 12 + 106 + 28 | 14 + 104 + 16 + 38 |
| SEQ ID NO: 198 | SEQ ID NO: 236 | SEQ ID NO: 274 | SEQ ID NO: 312 |

A peptide according to the present invention may be used in combination with any one or more different peptides according to the present invention.

A fragment, variant, fusion or derivative thereof, or a fusion of said fragment, variant or derivative thereof may comprise or consist of at least 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, 200, 300, 400, 500, 600, 700, 800, 900, 1000, 1100, 1200, 1300, 1400, 1500 or 1600 contiguous amino acids of the wildtype domain.

Peptides of the present invention, including fragments, variants, fusions or derivatives thereof, or a fusion of said fragment, variant or derivatives thereof, may be prepared using known protein engineering techniques. Protein engineering is the field that mimics natural evolution in the lab and aims for the development of proteins with designed features. Protein engineering techniques can be subdivided in mutagenesis and shuffling. In mutagenesis single or multiple nucleotides are substituted/deleted/added, whereas in shuffling longer DNA fragments are exchanged (Harayama, 1998). These techniques serve as tools for directed evolution of proteins, i.e., the creation of diversity by either mutagenesis or shuffling, followed by the selection of variants with desired features (Cobb et al., 2013; Lane & Seelig, 2014).

Both mutagenesis and shuffling can be performed in a rational manner. For site-directed mutagenesis this means the creation of site-directed point mutations, whereas for shuffling this implies the assembly of predefined gene fragments by restriction and ligation (Tee & Wong, 2013). In contrast, random mutagenesis creates randomly distributed mutations over the length of a selected DNA sequence. For example, error-prone PCR uses modified PCR parameters (DNA polymerase fidelity, dNTP, Mg²⁺ and Mn²⁺ concentrations) to introduce random mutations during amplification (Pritchard et al., 2005). Unlike mutagenesis, random shuffling is until today only possible for highly homologous genes (>70% similarity) (Nordwald et al., 2013). E.g., family shuffling uses DnaseI to randomly digest members of the same gene family followed by recombination between homologous regions and creation of a hybrid gene (Crameri et al., 1998).

An alternative method, Golden Gate shuffling, has been developed by (Engler et al., 2009) to shuffle both rationally and randomly fragments of homologous genes. Herein, Type IIs restriction enzymes inherent ability to directionally cut outside their recognition site is exploited to reduce both restriction and ligation to a one step, one pot reaction (Engler et al., 2009; Engler et al., 2008). This method can be used to simultaneously clone multiple fragments with a high efficiency. However, Golden Gate shuffling is limited in that only one variant of a peptide can be created at a time, which causes time limitations and is lab/labour intensive.

VersaTile shuffling has now been developed to efficiently shuffle and assemble fragments from non-homologous genes (Grimon et al., 2019), which can be used to develop libraries of peptides. Due to technological constraints the high-throughput rational and random assembly of nonhomologous genes was before not yet possible without reliance on time-consuming techniques, such as Golden Gate assembly. VersaTile shuffling, which is optimized for high yield in a shorter time, thus expands the possibilities of protein engineering towards the shuffling of non-homologous genes, hereby enabling the creation of totally new hybrid proteins with new protein functionalities. This novel technique can be exploited to generate and clone an unprecedented amount of new modular fusion peptides, for example those of the present invention. New fusion peptides falling within the scope of the present invention can therefore be generated starting from a repository of different Tiles (e.g. AMPs, LysB, LysA, optionally connected together via linkers).

Peptides of the present invention, including fragments, variants, fusions or derivatives thereof, or a fusion of said fragment, variant or derivatives thereof, may be purified using known purification techniques, thereby obtaining an isolated peptide, fragment, variant, fusion or derivative thereof, or an isolated fusion of said fragment, variant or derivative thereof. For example, purification may be by affinity chromatography, ion exchange chromatography, hydrophobic interactions, multimodal chromatography, gel filtration and/or size exclusion. In some embodiments, purification is by affinity chromatography, for example Immobilized Metal Affinity Chromatography (IMAC). The skilled person will appreciate that any suitable IMAC matrix can be used, for example a nickel column. In some embodiments, purification may be performed by using a HisTrap FF™ nickel column (GE Healthcare Bio-Sciences AB, Uppsala, Sweden).

At a minimum, the present invention refers to a peptide comprising three domains, wherein the domains have: (i) activity specific to a peptidoglycan or component thereof (a first domain); (ii) activity specific to an ester linkage (a second domain); and (iii) membrane permeabilising activity (a third domain). However, the peptide may further comprise additional domains that correspond to a first, second and/or third domain. For example, a peptide may comprise at least one first domain, at least one second domain, and at least one third domain. For example, a peptide may comprise two (or more) first domains without limitation to the number of second or third domains (e.g. 1, 2, or more) are present.

The arrangement of the domains may be in any order. Thus, the term “first position” in exemplary embodiments below merely refers to the first domain in a chain of domains that make up the peptides of the present invention. The nomenclature of first, second, third, fourth position and so on is merely to illustrate an optional order for the domains within a peptide.

For example, a peptide may comprise the first, second and third domains in any of the following orders (positions in order from N to C terminal):
(i) third domain-second domain-first domain (e.g. AMP-LysB-LysA)
(ii) third domain-first domain-second domain (e.g. AMP-LysA-LysB)
(iii) first domain-second domain-third domain (e.g. LysA-LysB-AMP)
(iv) second domain-first domain-third domain (e.g. LysB-LysA-AMP)
(v) first domain-third domain-second domain (e.g. LysA-AMP-LysB); or
(vi) second domain-third domain-first domain (e.g. LysB-AMP-LysA)

Additionally, as discussed in more detail below, multiple copies or versions of each domain may be present, for example there may be multiple first domains in the peptide and/or multiple second domains in the peptide and/or multiple third domains in the peptide.

Thus, in example embodiments, a peptide may be comprised of the following domains from N-terminal to C-terminal:

| First | Second | Third | Fourth | Fifth |
|---|---|---|---|---|
| position | position | position | position | position |
| Third domain | Second domain | First domain | — | — |
| (e.g. AMP) | (e.g. LysB) | (e.g. LysA) | | |
| Third domain | Second domain | First domain | Fourth domain | — |
| (e.g. AMP) | (e.g. LysB) | (e.g. LysA) | (e.g. PTD) | |
| Third domain | First domain | Second domain | — | — |
| (e.g. AMP) | (e.g. LysA) | (e.g. LysB) | | |
| First domain | Third domain | Third domain | Second domain | — |
| (e.g. LysA) | (e.g. AMP) | (e.g. AMP) | (e.g. LysB) | |
| Fourth domain | Third domain | First domain | Second domain | Third domain |
| (e.g. PTD) | (e.g. AMP) | (e.g. LysA) | (e.g. LysB) | (e.g. AMP) |
| Second domain | Fourth domain | Third domain | First domain | — |
| (e.g. LysB) | (e.g. PTD) | (e.g. AMP) | (e.g. LysA) | |
| Third domain | Third domain | Third domain | First domain | Second domain |
| (e.g. AMP) | (e.g. AMP) | (e.g. AMP) | (e.g. LysA) | (e.g. LysB) |
| First domain | Second domain | First domain | Third domain | Fourth domain |
| (e.g. LysA) | (e.g. LysB) | (e.g. LysA) | (e.g. AMP) | (e.g. PTD) |
| Second domain | First domain | Third domain | — | — |
| (e.g. LysB) | (e.g. LysA) | (e.g. AMP) | | |
| First domain | Second domain | Third domain | — | — |
| (e.g. LysA) | (e.g. LysB) | (e.g. AMP) | | |
| Second domain | Third domain | First domain | — | — |
| (e.g. LysB) | (e.g. AMP) | (e.g. LysA) | | |

Accordingly, the domains are interchangeable within the peptides provided the minimum three domains are present, as described above. Additional domains, including repeats of the type of domain, or linkers may be introduced at the N-terminus, C-terminus, and/or between any of the first, second, third, fourth or fifth positions above. In some embodiments, a peptide may comprise a linker between every domain in each position above, but not present at the C- or N-terminus. In some embodiments, a peptide may comprise no linkers. In some embodiments, a peptide may comprise only one linker between two domains. In some embodiments, the LysA may be in the form of an EAD derived from the LysA. Exemplary EAD include amino acid sequences corresponding to SEQ ID NOS: 326-338, as encoded by the nucleic acid sequences corresponding to SEQ ID NOs: 313-325, respectively.

In some embodiments, the peptide may comprise at least one first domain (e.g. at least one LysA), at least one second domain (e.g. LysB) and at least one third domain (membrane permeabiliser, e.g. an AMP or holin). In some embodiments, the peptide may comprise at least one first domain (e.g. LysA), at least one second domain (e.g. LysB), and at least one third domain (membrane permeabiliser), optionally further comprising at least one fourth domain (PTD/CPP). Either of these embodiments, or any further embodiments described herein, may further comprise at least one linker at any position between domains.

In some embodiments, a peptide may comprise multiple iterations of a particular domain (or of multiple domains, including multiple iterations of all domains). For example, a peptide may comprise 1, 2, 3, 4, 5, 6, 7, 8, 9, 10, or more domains with activity specific to a peptidoglycan or component thereof (first domain); 1, 2, 3, 4, 5, 6, 7, 8, 9, 10, or more domains with activity specific to an ester linkage (second domain); and/or 1, 2, 3, 4, 5, 6, 7, 8, 9, 10, or more domains with membrane permeabilising activity (third domain); and/or 1, 2, 3, 4, 5, 6, 7, 8, 9, 10, or more protein transduction domains (PTDs). Each domain may be in any order and thus fused to any other domain (either directly or indirectly). By “directly” we include the meaning that the domains are fused to each other without a linker between each domain. By “indirectly” we include the meaning that the domains are fused to each other either via a linker or via at least one other domain in-between. For example, at least one LysA could be indirectly linked to at least one AMP via at least one LysB, or via at least one linker.

When multiple iterations of a particular domain are present (e.g. multiple first domains) these may be multiple copies of the same domain (e.g. multiple LysA domains) or different ‘first domains’ which differ in sequence/structure, but all share the common functional property of activity specific to a peptidoglycan or component thereof (e.g. multiple different EADs).

In one embodiment, the peptide comprises of the following domains from N-terminal to C-terminal: third domain (e.g. AMP), second domain (e.g. LysB), first domain (e.g. LysA). In the same or different embodiments, the peptide may also comprise a linker between the second and first domain and/or may comprise one or more PTD domains at the C terminal end of the arrangement. For example, the peptide may comprise AMP-LysB-LysA, AMP-LysB-LysA-PTDs, AMP-LysB-Linker-LysA or AMP-LysB-Linker-LysA-PTDs.

In one embodiment, the peptide does not comprise the following domains from N-terminal to C-terminal: third domain (e.g. AMP), second domain (e.g. LysB), first domain (e.g. LysA). In another embodiment the peptide does not comprise the following domains from N-terminal to C-terminal: third domain (e.g. AMP), second domain (e.g. LysB), first domain (e.g. LysA) wherein there is a linker between the second and first domain. Thus, in one embodiment the peptide does not comprise the following arrangement: AMP-LysB-Linker-EAD. In another embodiment, the peptide does not comprise the following arrangement: AMP-LysB-EAD.

Accordingly in some embodiments, the peptide does not comprise one or more of the following arrangements:
(a) AMP-LysB-Linker-LysA
(b) AMP-LysB-LysA
(c) AMP-LysB-Linker-EAD
(d) AMP-LysB-EAD
(e) AMP-LysB-Linker-LysA-PTD
(f) AMP-LysB-LysA-PTD.

In some embodiments, the peptides of the invention do not exceed 120 kDa. For example, a peptide may be about 10 kDa, 20 kDa, 30 kDa, 40 kDa, 50 kDa, 60 kDa, 70 kDa, 80 kDa, 90 kDa, 100 kDa, 110 kDa, or 120 kDa.

As specified above, peptides of the present invention optionally further comprise at least one linker (i.e. linker peptides). Linkers are comprised of at least one amino acid, more typically a chain of two or more amino acids, that are fused to and between domains of peptides. In some embodiments, a linker may not be required at all. In some embodiments, a linker may be required only between particular domains. For example, a linker may be required to prevent steric hindrance between two domains (i.e. where an entire LysA and LysB are present, depending on their sizes, a linker may be beneficial, albeit not necessarily compulsory, to retaining their function). In some embodiments, where an entire LysA and/or LysB is included, a linker may be of higher importance (especially for certain LysA that are known to be particularly large in size) to reduce risk of steric hindrance.

Any of the domains described herein that are fused to or between any other domain may be considered a linker. For example, in a fusion peptide comprised of LysA-LysB-AMP, the LysB may be considered a linker between LysA and AMP, and/or the AMP or LysA may be considered a linker attached to LysB (which optionally may link LysB to further domains and/or components/portions thereof). In some embodiments, linkers do not correspond to any of the domains (i.e. the domains with specified activity), but instead correspond to further amino acids or stretches thereof. In another example, in a fusion peptide comprised of LysB-AMP-LysA, the AMP may be considered a linker between LysB and LysA, and/or the LysB or LysA may be considered a linker attached to AMP (which optionally may link AMP to further domains and/or components/portions thereof).

Accordingly, the purpose of a linker is to connect to a domain. In some embodiments, at least one linker connects:
1. the first domain to the second domain;
2. the second domain to the third domain;
3. the first domain to the third domain;
4. the first domain to the fourth domain;
5. the second domain to the fourth domain;
6. the third domain to the fourth domain; and/or
7. a domain to a further linker.

Linkers may be present in tandem (either as repeats of the same linker, or as two or more linkers connected to each other). All linkers of the present invention can be in either orientation. For example, where a sequence is provided for a linker, that sequence may be fused to any other domain or linker by its N- and/or C-terminus, and so any sequence written N- to C-terminally would equally be valid written C- to N-terminally in a fusion peptide.

Linkers can be of any length. In some embodiments, linkers are between 1 and 100 amino acids in length, for example, between 5 and 50 amino acids in length, between 10 and 30 amino acids in length, and/or between 15 and 20 amino acids in length. Where more than one linker is present in a peptide, each linker may correspond to the same sequence or different sequences. Preferably, the linker is of a length that does not disrupt any one or more of the beneficial activities of the peptide of the invention.

In one embodiment, linkers that are not domains may comprise any one or more (or multiple repeats) of the amino acid sequences (in either orientation) selected from any one or more of SEQ ID NOs: 104, 106, 108, 110, 112 or 114; or said linkers may be encoded by any one or more of the nucleotide sequences of SEQ ID NO: 103, 105, 107, 109, 111 or 113, as follows:

Length: 18
Type: DNA
Other information: Linker-A (also known as “L1”)
Sequence ID No: 103
GCGGGTGCGGGTGCGGGT
Length: 6
Molecular weight: 0.4 kDa
Type: Protein
Feature: Flexible short linker
Other information: Linker-A (also known as “L1”)
Sequence ID No: 104
AGAGAG
Length: 27
Type: DNA
Other information: Linker-B (also known as “L4”)
Sequence ID No: 105
CTGTCTCGTTTCTTCCACGCGGAACTG
Length: 9
Molecular weight: 1.119 kDa
Type: Protein
Feature: Rigid short helix
Other information: Linker-B (also known as “L4”)
Sequence ID No: 106
LSRFFHAEL
Length: 42
Type: DNA
Other information: Linker-C (also known as “L5”)
Sequence ID No: 107
GTTTTCAACCAGCGTAAAGAACACAAAGGTTACATGCTGGCG
Length: 14
Molecular weight: 1.721 kDa
Type: Protein
Feature: Rigid long helix
Other information: Linker-C (also known as “L5”)
Sequence ID No: 108
VFNQRKEHKGYMLA
Length: 48
Type: DNA
Other information: Linker-D (also known as “L6”)
Sequence ID No: 109
ATCCCGCAGGGTCGTTCTCACCCGGTTCAGCCGTACCCGGGTGCGTTC
Length: 16
Molecular weight: 1.75 kDa
Type: Protein
Feature: Rigid long coil
Other information: Linker-D (also known as “L6”)
Sequence ID No: 110
IPQGRSHPVQPYPGAF
Length: 18
Type: DNA
Other information: Linker-E (also known as “L7”)
Sequence ID No: 111
CCGGCGGTTCCGCCGCCG
Length: 6
Molecular weight: 0.576 kDa
Type: Protein
Feature: Rigid short coil
Other information: Linker-E (also known as “L7”)
Sequence ID No: 112
PAVPPP
Length: 39
Type: DNA
Other information: Linker-F (also known as “L8”)
Sequence ID No: 113
GAAGCGGCGGCGAAAGAAGCGGCGAAAGAAGCGGCGAAA
Length: 13
Molecular weight: 1.287 kDa
Type: Protein
Feature: Rigid optimal helix
Other information: Linker-F (also known as “L8”)
Sequence ID No: 114
EAAAKEAAKEAAK

Alternatively or additionally, the linkers may also be variants, fragments, fusions or derivatives of the sequences given above, or fusions of said fragments, variants and derivatives thereof.

In some embodiments, the peptide comprises at least one lysin as defined herein (e.g. an endolysin, such as LysA, and/or an enzyme with lipolytic activity, such as LysB; or a portion of any of the aforementioned (e.g. an EAD)) that is effective against the phylum Actinobacteria; derived from the class Actinobacteridae; derived from the order Actinomycetales, and/or Bifidobacteriales. Optionally wherein at least one lysin is effective against a subclass and/or family selected from the list consisting of: Actinomycineae: Actinomycetaceae (Actinomyces, Mobiluncus); Corynebacterineae: Mycobacteriaceae (Mycobacterium), Nocardiaceae; Frankineae: Frankiaceae; Micrococcineae: Brevibacteriaceae; Propionibacteriaceae (Propionibacterium); Bifidobacteriaceae (Bifidobacterium, Falcivibrio, Gardnerella); Acidimicrobidae, Coriobacteridae, Rubrobacteridae, Sphaerobacteridae.

In some embodiments, at least one domain defined herein, such as a lysin as defined herein (e.g. an endolysin, such as LysA, or a portion of any of the aforementioned (e.g. an EAD)) may be derived from a bacteriophage capable of infecting gram positive bacteria and/or gram negative bacteria and/or mycobacteria. In some embodiments, at least one domain defined herein, such as a lysin as defined herein (e.g. an enzyme with lipolytic activity, such as LysB; or a portion of any of the aforementioned (e.g. an EAD)) may be derived from a bacteriophage capable of infecting mycobacteria.

In some embodiments, at least one domain defined herein, such as a lysin as defined herein (e.g. an endolysin, such as LysA, and/or an enzyme with lipolytic activity, such as LysB; or a portion of any of the aforementioned (e.g. an EAD)) may comprise or consist of the amino acid sequence corresponding to any one of SEQ ID NOS: 1-32.

In one embodiment one or more domains are derived from a mycobacteriophage capable of infecting mycobacterium. Mycobacteriophages may be in the form of a prophage, wherein the genetic material of a bacteriophage is incorporated into a host (bacterium), and the host is able to produce phages. The term “mycobacteriophage” therefore includes phages produced by a prophage of said mycobacteriophage. Thus, in some embodiments, the peptide comprises at least one domain defined here, such as a lysin as defined herein (e.g. an endolysin, such as LysA, and/or an enzyme with lipolytic activity, such as LysB; or a portion of any of the aforementioned (e.g. an EAD)) that is derived from a mycobacteriophage or a Mycobacterium prophage selected from the group consisting of: TM4, D29, L5, Bxz2, Saal, Enkosi, Ms6, Omega, Obama12, Echild, DS6A, Pumpkin or any other mycobacteriophage as listed in The Actinobacteriophage Database (see phagesdb.org/hosts/genera/1/). Peptides described herein may comprise lysins and/or components/portions thereof all derived from the same mycobacteriophage or from multiple mycobacteriophages. For example, a peptide may be a fusion of domains derived from at least 2 mycobacteriophages, optionally at least 3, 4, 5, 6, 7, 8, 9, 10 or more mycobacteriophages. For example, a peptide may be a fusion of at least one LysA (and/or portion thereof) derived from TM4 and at least one LysB (and/or portion thereof) derived from D29.

Peptide of the present invention have antimicrobial properties. In one embodiment, the peptides are for use treating bacterial infection, for example mycobacterial infection. The peptides may be used for treating any bacterial infection caused by a bacterial species with mycolic acids present in the cell envelope or substantially free from mycolic acid. Accordingly, in some embodiments, the peptide is capable of disrupting the cell membrane of gram-positive bacteria that have mycolic acids in their cell membrane. In some embodiments, the peptides are capable of disrupting the cell membrane of mycobacteria. Species of Mycobacterium include, but are not limited to: Mycobacterium abscessus, Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium kansasii, Mycobacterium terrae, Mycobacterium nonchromogenicum, Mycobacterium gordonae, and Mycobacterium triviale, and non-tuberculosis mycobacteria (NTM). In one preferable embodiment, the peptides are capable of disrupting the cell membrane of Mycobacterium abscessus.

In some embodiments, the bacterium (e.g. mycobacterium) may be drug resistant (e.g. multidrug resistant). For example, the target bacteria may be resistant to at least one drug, optionally at least 2, 3, 4, 5, 6, 7, 8, 9, 10 or more drugs. By “drug resistant” we include the meaning that the bacterium (e.g. mycobacterium) is not susceptible to treatment with an antibacterial (or antimycobacterial) agent. Accordingly, the bacteria may be multidrug-resistant (MDR), extremely drug-resistant (XDR), or totally drug-resistant (TDR). The uses described herein may facilitate the function of further therapeutic agents. Drug resistance may arise due to specific components and/or configurations of the cell wall. Drug resistant strains of bacteria, for example, tend to have thicker cell walls. Thus, peptides and uses thereof described herein may affect the cell envelope in such a way as to facilitate the function of a further therapeutic agent (for example, an agent that acts intracellularly to the bacteria). However, the composition of MDR, XDR and/or TDR strains remains largely the same. Accordingly, the effectiveness of various peptides herein against the MDR strain M. abscessus means it is likely that other drug resistant strains would also be susceptible. In one embodiment, the peptide is effective in treating the MDR strain M. abscessus. For example, the peptide may cause disruption of the cell membrane of the MDR strain M. abscessus, thereby resulting in osmotic shock and death of the mycobacterium.

Tuberculosis, for example, which results from an infection with Mycobacterium tuberculosis, can be cured with a combination of first-line drugs taken daily for several months. MDR TB occurs when a Mycobacterium tuberculosis strain is resistant to isoniazid and rifampin, two of the most powerful first-line drugs. To cure MDR TB, healthcare providers must turn to a combination of second-line drugs, several of which are shown here. Second-line drugs may have more side effects, the treatment may last much longer, and the cost may be up to 100 times more than first-line therapy. MDR TB strains can also grow resistant to second-line drugs, further complicating treatment. XDR TB occurs when a Mycobacterium tuberculosis strain is resistant to isoniazid and rifampin, two of the most powerful first-line drugs, as well as key drugs of the second line regimen-any fluoroquinolone and at least one of the three injectable drugs shown above. XDR TB strains may also be resistant to additional drugs, greatly complicating therapy.

In some embodiments, the peptides are for use in treating co-infections. For example, the peptides may be for use in treating someone that has a viral infection who may be more susceptible to, or already infected with, a bacterial (e.g. infection) infection. In some embodiments, the viral infection is selected from one or more of the groups consisting of: coronavirus, HIV, influenza, viral pneumonia, enterovirus and norovirus.

In some embodiments, the peptide of the present invention is not the following: a peptide which comprises an outer membrane acting biologic and a mycobacterial chemotherapeutic, but which lacks a domain with specific activity to a peptidoglycan layer.

In other embodiments, the peptide of the invention is not a peptide which has an outer membrane acting biologic that is LysB or D29 phage LysB and which further comprises a mycobacterial chemotherapeutic, but which lacks a domain with specific activity to a peptidoglycan layer.

In yet other embodiments, the peptide of the invention is not a peptide which has an outer membrane acting biologic that is LysB or D29 phage LysB and which further comprises a mycobacterial chemotherapeutic, but which lacks a LysA domain with specific activity to a peptidoglycan layer.

In another embodiments, the peptides of the present invention are not:
a fusion protein comprising a LysA and AMP (but not comprising a LysB) or
a fusion peptide comprising a LysB and AMP (but not comprising a LysA).

Thus in one embodiment any peptide which does not comprise, as a single peptide, at least a LysA, a LysB and an AMP, is excluded.

In a second aspect of the invention, the peptide may be in the form of a composition (e.g. a pharmaceutical composition. Accordingly, a composition comprising a peptide may be any one or more of the peptides as defined herein, in any combination or permutation, provided that the composition retains the intended activity (or at least a portion of the intended activity). In some embodiments a composition may comprise a further agent, for example a pharmaceutically acceptable excipient, diluent, carrier, buffer and/or adjuvant.

Additional compounds may also be included in the pharmaceutical compositions, such as other peptides, low molecular weight immunomodulating agents, receptor agonists and antagonists, and antimicrobial agents. Other examples include chelating agents such as EDTA, citrate, EGTA or glutathione.

The pharmaceutical compositions may be prepared in a manner known in the art that is sufficiently storage stable and suitable for administration to humans and animals. The pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the biological activity of the active ingredients, i.e. the antimicrobial polypeptide(s) of the composition. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000)).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide of the composition. The adjuvant may be one or more of colloidal silver, or zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids, detergents and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient (applies to A-C as well) from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical composition may also contain one or more mono- or di-saccharides such as xylitol, sorbitol, mannitol, lactitiol, isomalt, maltitol, glycerol or xylosides, and/or monoacylglycerols, such as monolaurin. The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

The pharmaceutical compositions of the invention may also be in the form of a liposome, in which the peptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly (ethylene glycol) in the polar headgroup for prolonging bloodstream circulation time. Preparation of such liposomal formulations can be found in for example U.S. Pat. No. 4,235,871.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 213 303.

The pharmaceutical compositions of the invention may also be formulated with micellar systems formed by surfactants and block copolymers, preferably those containing poly(ethylene oxide) moieties for prolonging bloodstream circulation time.

The pharmaceutical compositions of the invention may also be in the form of polymer gels, where polymers such as starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, chitosan, carrageenan, hyaluronic acid and derivatives thereof, polyacrylic acid, polyvinyl imidazole, polysulphonate, polyethylenglycol/polyethylene oxide, polyethylene-oxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

The pharmaceutical composition may also include ions and a defined pH for potentiation of action of the activities of peptides (e.g. the activity as described above for any of the domains).

The above compositions of the invention may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the pharmaceutical compositions and/or peptides of the invention may be administered locally or systemically. Routes of administration include topical (e.g. ophthalmic), ocular, nasal, pulmonary, buccal, parenteral (intravenous, subcutaneous, and intramuscular), oral, vaginal and rectal. In one embodiment the administration may be respiratory, for example by inhalation either orally or nasally. Such administration is also referred to as inhalation or pulmonary administration. Also, administration from implants is possible. Suitable preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts, gels, ointments, dispersions, suspensions, creams, aerosols, wafers, droplets or injectable solution in ampoule form and also preparations with protracted release of active compounds, in whose preparation excipients, diluents, adjuvants or carriers are customarily used as described above. The pharmaceutical composition and/or peptide may also be provided in bandages, plasters or in sutures or the like.

In some embodiments, the pharmaceutical composition and/or peptide is suitable for oral administration, parenteral administration and/or topical administration. For example, the pharmaceutical composition may be suitable for topical administration (e.g. ophthalmic administration, in the form of a spray, lotion, paste or drops etc.). The route of administration may depend on the type of infection. For example, inhalation/pulmonary routes of administration are more beneficial for treating TB infection.

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the activity of the compound, manner of administration, nature and severity of the disorder, age and body weight of the patient, whereby different and/or adapted doses may be needed. The administration of the dose can be carried out both by single administration in the form of an individual dose unit or else several smaller dose units and also by multiple administrations of subdivided doses at specific intervals.

The pharmaceutical compositions of the invention may be administered alone or in combination with other therapeutic agents, such as additional antibiotic, anti-inflammatory, immunosuppressive, proteases, vasoactive and/or antiseptic agents (such as anti-bacterial agents, anti-fungicides, anti-viral agents, and anti-parasitic agents). Examples of suitable additional antibiotic agents include penicillins, cephalosporins, carbacephems, cephamycins, carbapenems, monobactams, aminoglycosides, glycopeptides, quinolones, tetracyclines, macrolides, and fluoroquinolones. Antiseptic agents include iodine, silver, copper, chlorhexidine, polyhexanide and other biguanides, chitosan, acetic acid, and hydrogen peroxide. Likewise, the pharmaceutical compositions may also contain anti-inflammatory drugs, such as steroids and macrolactam derivatives. The further therapeutic agent may be selected depending on the target bacterium, for example the combination therapy may be with anti-TB drugs if TB is the intended target.

Further therapeutic agents may be mycobacterial chemotherapeutics that are antibiotics, including any one or more agent selected from the list consisting of: isoniazid, pyrazinamide, ethambutol, or rifampin, fluoroquinolones (e.g. ciprofloxacin, levofloxacin, moxifloxacin), cyclic peptides (e.g. capreomycin, viomycin, enviomycin), thioamides (e.g. ethionamide, prothionamide), cycloserine, terizidone, an aminoglycoside, PAS, kanamycin, capreomycin, amikacin, streptomycin, macrolide, a β-lactam, a β-lactamase inhibitor, clavulanic acid, trimethoprim, or sulfamethoxazole, clarithromycin, rifampicin, rifabutin, amikacin, azithromycin, or moxifloxacin, diarylquinoline, bedaquiline, dedaquiline, TMC207, nitroimidazoles (including PA-824 and OPC-67683), oxazolidinones (including linezolid, sutezolid, and AZD5847), BTZ043, and SQ109.

Combination therapies as described herein may have: (i) enhanced potency, which may allow the dose of peptides of the present invention to be attenuated; (ii) a low (or lower) chance (e.g. compared with peptides of the present invention alone, and/or compared with known therapies, including known combination therapies) of having resistance developed against them; (ii) increased specificity (for example, due to the CBD derived from LysA improving specificity to cell walls of interest, including mycobacterial cell walls); and/or (iv) improved activity intracellularly (e.g. due to fusion with a PTD as described herein).

Such additional therapeutic agents may be incorporated as part of the same pharmaceutical composition or may be administered separately. Additional therapeutic agents can be administered simultaneously, sequentially and/or separately, either before, after and/or during administration of a pharmaceutical composition described herein.

It will be appreciated by persons skilled in the art that the peptides of the invention, compositions of the invention, and/or pharmaceutical compositions thereof, may be applied to devices (such as medical devices) and other products the implantation into or application of which to the human or animal body is associated with the risk of infection by a microbial agent. The purpose of applying peptides, compositions and/or pharmaceutical compositions of the invention may be to protect devices (e.g. medical devices) from mycobacterial infection. Accordingly, in some embodiments, the peptides, compositions and/or pharmaceutical compositions of the invention may be at least a surface coating on a device. Alternatively, or additionally, a device comprising a peptide, composition and/or pharmaceutical composition of the invention may be for use in recognition, prevention, monitoring, treatment and/or alleviation of mycobacteria or mycobacterial diseases.

Peptides, compositions and/or pharmaceutical compositions of the invention may be in the form of nanoparticles, hydrogels, creams, ointments and/or wafers, for example for topical application.

In some embodiments, the peptides, compositions and/or pharmaceutical compositions of the invention may be for use in water treatment to reduce (or remove) mycobacteria. For example, a water supply can be treated with a peptide, composition and/or pharmaceutical composition after decanting the water into a vessel; and/or a filter could be applied to a water system that treats water passing through said filter, wherein the filter comprises a peptide, composition and/or pharmaceutical composition of the invention.

In some embodiments, the peptides and/or compositions described herein are for use in medicine, including veterinary medicine.

In some embodiments, the peptides and/or compositions described herein are for cosmetic purposes. For example, the peptides and/or compositions described herein may be prepared as a cosmetic. Such a cosmetic preparation may be in a formulation as described for compositions herein, including being suitable for any administration route as described herein. For example, a cosmetic composition may have activity against Propionibacterium acnes, and thus may be useful in treating acne.

In some embodiments, the peptides and/or compositions described herein may be components within a kit. A kit may further comprise instructions for use and/or constitution of peptides and/or composition.

In some embodiments, the peptides and/or compositions described herein may be for use in a diagnostic test, for example a diagnostic test for identifying mycobacterial infection. Diagnostic tests may be on a sample derived from a subject or on inanimate objects.

In some embodiments, the peptides and/or compositions described herein may be for use in the preparation of vaccines for use in treating or preventing disease in an organism, for example in a human, sheep, cow, dog, pig, or other farm animal. Alternatively, the vaccine may be prepared from a nucleic acid that encodes a peptide (or peptides) as described herein.

In some embodiments, the peptides and/or compositions described herein may be for use in:
1. promoting wound healing;
2. treating infection, optionally wherein the infection is by a bacterium with mycolic acids in their cell wall, for example mycobacterial infection, such as M. abscessus, or when the infection is caused by non-tuberculous mycobacteria (NTM);
3. treating skin/oral/dental infections
4. ocular, skin, soft tissue, oral and/or dental procedures;
5. sterilisation (e.g. sterilisation via biocidal activity, wherein the peptide is acting as a biocide);
6. treating granulomas and/or
7. treating biofilms.

For example, in one embodiment the peptides and/or compositions described herein may be used for treating a skin or soft tissue infection caused by non-tuberculosis mycobacteria (NTM).

As described above, in one embodiment, the peptides and/or compositions of the invention may be used in treating tuberculosis (TB). In one embodiment the tuberculosis to be treated may be active. In one embodiment the tuberculosis to be treated may be latent (or dormant) tuberculosis.

The treatment of “infection” as described herein may refer to an external and/or internal infection, and instances where co-infection of one pathogen may be more likely due to infection with an initial pathogen. For example, the infection may be internal to a subject in need of treatment, e.g. a patient with M. abscessus infection. Additionally, or alternatively, the infection may be on an external object, such as an object suspected or confirmed to be infected with M. abscessus and so in need of sterilisation from said infection.

By “biofilm” we mean any group of microorganisms in which cells stick to each other on a surface, such as in a complex structure, thereby forming a biofilm. For example, the culture (or natural growth) of mycobacteria may result in a biofilm forming, whereby many mycobacterial cells are stuck together on a surface. In some embodiments, the peptides are capable of breaking up biofilms. For example, the peptides may disrupt the interactions between mycobacterial cells that have formed a biofilm, and/or may prevent new formation of biofilms from existing mycobacteria on a surface (e.g. by pre-sterilising with peptides).

Formation of a biofilm begins with the attachment of free-floating microorganisms (e.g. mycobacteria) to a surface. These first colonists adhere to the surface initially through weak, reversible adhesion and, if not immediately separated from the surface, can anchor more permanently using cell adhesion structures (such as pili). Some species are not able to attach to a surface on their own but are sometimes able to anchor themselves to the matrix or directly to earlier colonists. Once colonization has begun, the biofilm grows through a combination of cell division and recruitment. Polysaccharide matrices typically enclose bacterial biofilms. The final stage of biofilm formation is known as dispersion and is the stage in which the biofilm is established and may only change in shape and size.

In one embodiment, a biofilm may comprise, consist essentially of, or consist of, multiple strains of microbial cells (e.g. mycobacterial cells) growing in a biofilm. Optionally, a biofilm may comprise, consist essentially of, or consist of, one species or strain of microbial (e.g. mycobacterial) cells.

In an alternative option, a biofilm may comprise, consist essentially of, or consist of, more than one species or strains of mycobacterial cell, such as up to at least 2, 3, 4, 5, 6, 7, 8, 9, 10, 15, 20, 30, 40, 50, 60 70, 80, 90, 100, 150, 200, 250, 300, 350, 400, 450, 500, 600, 700, 800, 900, 1000 or more different species or strains of mycobacterial cell.

Accordingly, peptides, compositions and methods for inhibiting, reducing, or removing biofilm forming mycobacteria and mycobacterial infections are provided. In some embodiments, peptides or composition may further comprise an extra enzyme that is a biofilm degrader. For example, a biofilm degrader may be selected from the group consisting of depolymerases, glycoside hydrolases, lipases, esterases, nucleases and proteases. In some embodiments, a method for inhibiting, reducing or removing biofilm forming mycobacteria may comprise the step of treating a surface with a peptide or composition described herein, optionally further comprising the step of treating with an enzyme that is a biofilm degrader (sequentially, simultaneously and/or subsequently to the peptide or composition).

It will be apparent to the skilled person that as the invention provides peptides, the invention also provides corresponding polynucleotides that comprise or consist of a sequence that encodes the peptides of the invention (or domains thereof which are subsequently fused to a peptide of the present invention). The invention provides a DNA polynucleotide that comprises or consists of a sequence that encodes at least one peptide of the invention or at least one domain thereof. The invention also provides an RNA polynucleotide that comprises or consists of a sequence that encodes at least one peptide of the invention or at least one domain thereof. The skilled person will appreciate that a polynucleotide, for example a DNA or RNA polynucleotide, may comprise one or more modifications, for example a phosphorothioate modification. The polynucleotide may also comprise one or more other features, for example a promoter, terminator, or a tag for instance, for example the features typical of an expression cassette.

Nucleic acids of the present invention may not correspond to mycobacteriophage lysis cassettes. Mycobacteriophage lysis cassettes encode a single LysA or LysB. Some mycobacteriophages do not have lysis cassettes that encode a LysB. However, lysis cassettes capable of encoding a peptide comprising at least one LysA (or component thereof) and at least one LysB (or component thereof) do not exist in nature, let alone a peptide further comprising a membrane permeabiliser domain. Similarly, mycobacteriophages that product LysA and LysB peptides do so as separate peptides, and not combined in the way of the peptides of the present invention.

The invention also provides a nucleic acid vector comprising the nucleic acid of the invention. The skilled person will understand that by nucleic acid vector we include the meaning of a plasmid, phage, artificial chromosome or other nucleic acid structure used to deliver or express at least one peptide or at least one domain thereof. The artificial chromosome may be any artificial chromosome and may be selected from, for example, a bacterial artificial chromosome (BAC), a yeast artificial chromosome (YAC), and a Human artificial chromosome (HAC).

The invention also provides a cell that comprises at least one peptide of the invention (or at least one domain thereof), at least one polynucleotide of the invention and/or at least one vector of the invention. The cell of the invention has two main uses, amongst others: i) manufacture of the cyclic peptides or viral vectors comprising at least one peptide of the invention, for example; and ii) medical uses for example screening for suitable peptides for particular situations, or as a therapeutic cell.

In one embodiment, the cell is a cell that is used in the commercial, large scale manufacture of the peptides of the invention, for example is a bacterial cell such as E. coli, or is a yeast cell such as P. pastoris, or a cell derived from a plant, virus, insect or otherwise.

In another embodiment the cell is a cell that either is a direct “diseased” cell, for example taken from a biopsy from a patient. In another embodiment the cell is a cell that is intended to mimic or model a particular disease state. Such cells can be used to screen for appropriate peptides that are suitable for use in particular therapeutic situations.

Since the peptides of the invention are able to disrupt particular microbial cell walls, for example mycobacterial cell walls, it will be apparent to the skilled person that the peptides of the invention, the polynucleotides of the invention and/or the vectors of the invention have use in the treatment and/or prevention of diseases, disorders or conditions. For example, in one embodiment the peptides of the invention are useful in the treatment or prevention of a disease, disorder or condition that is associated with and/or caused by bacterial, for example mycobacterial, infection.

The peptides and/or compositions of the present invention may be for use in a method (e.g. an in vitro method) of lysing and/or killing bacteria (e.g. mycobacteria), and/or in a method of reducing the growth and/or viability of said bacteria.

In some embodiments is provided a method (e.g. an in vitro method) of lysing and/or killing bacteria (e.g. mycobacteria), and/or in a method of reducing the growth and/or viability of said bacteria, with any one or more of the previously described embodiments (e.g. peptides and/or compositions described herein).

Lysing and/or killing bacteria (e.g. mycobacteria) and/or reducing the growth and/or viability of bacteria may be assessed, for example, based on percentage growth inhibition.

The percentage growth inhibition can be calculated following an assessment of optical density using methods known to the skilled person and as demonstrated in the Examples, wherein a lower optical density may indicate a lower level of bacteria. Thus, in some embodiments, the peptide may have a percentage growth inhibition of at least 50%, 60%, 70%, 80%, 85%, 90%, 95%, 96%, 97%, 98%, 99%, or of 100%. Preferably, the peptides have a percentage growth inhibition of at least 90%; more preferably at least 95%. For further details of percentage growth inhibition measurements, see Tenland et al., 2018.

Alternatively, or additionally, the reduction in growth or lysing/killing of bacteria may be measured according to a scaled scoring system. In one embodiment of such a scoring system, different scores from ‘+++’, ‘+’, ‘+’ and ‘−’ are assigned, wherein ‘+++’ is indicative of the most bacterial growth inhibition, and ‘−’ is indicative of the lowest level of inhibition, or no level of growth inhibition. In one embodiment, assessment is based on OD655 nm values (representative of bacterial growth in a plate) as follows: hits are ranked as ‘+++’ if no bacterial growth was observed; hits are ranked as ‘++’ if they exhibit bacterial growth with OD655 nm between 0 and 0.1; hits are ranked as ‘+’ if they exhibit bacterial growth with OD655 nm between 0.1 and 0.2; and finally hits are ranked as ‘-’ if they exhibit bacterial growth with OD655 nm larger than 0.2. The OD655 nm values can be corrected for background OD655 nm of the medium. For further details of such a scoring system see Gerstmans et al., 2020. Thus in one embodiment the peptides of the invention may be described as being capable of lysing/killing bacteria if they are classed as ‘+++’, or alternatively the peptides may be described as being capable of lysing/killing bacteria if they are classed as ‘+++’ or ‘++’, or alternatively the peptides may be described as being capable of lysing/killing bacteria if they are classed as ‘+++’, ‘+’ or ‘+’.

Peptides may be classed as ‘hits’ i.e. peptides having a predefined level of bacterial killing activity, if the OD of bacterial growth in a plate is less than 0.1, as described above and further in the Examples of the present application. Alternatively, peptides may be classed as hits if the OD is less than 0.15, 0.20, 0.25, 0.30, 0.35, 0.40, 0.45 or 0.50.

Such bacterial growth inhibition may also be measured relative to a positive control (e.g. a plate well wherein the bacteria is allowed to grow without application of the peptides of the invention). Accordingly, any clones expressing measurable inhibition (i.e. significantly lower OD than the control) may be classed as having bacterial killing/lysing/growth inhibition effect.

Accordingly, the present application also provides aspects according to the following numbered paragraphs.
1. A peptide comprising:
   (i) a first domain with activity specific to a peptidoglycan or component thereof, and
   (ii) a second domain with activity specific to an ester linkage; and
   (iii) a third domain with membrane permeabilising, destabilizing and/or disrupting activity.
2. The peptide according to paragraph 1, further comprising a fourth domain that is at least one protein transduction domain (PTD).
3. The peptide according to paragraph 1 or 2, wherein the first domain is a lysin, optionally an endolysin; a lysozyme; and/or an autolysin.
4. The peptide according to paragraph 3, wherein the endolysin is selected from the group consisting of structural endolysins, modular endolysins, globular endolysins.
5. The peptide according to paragraph 3 or 4, wherein the endolysin is a peptidoglycan hydrolase enzyme.
6. The peptide according to any preceding paragraph, wherein the first domain is a LysA and/or at least one portion thereof, optionally wherein the portion thereof is at least one enzymatically active domain (EAD).
7. The peptide according to any preceding paragraph, wherein the first domain comprises or consists of an amino sequence selected from any one of SEQ ID NOS: 2, 4, 6, 8, 10, 12, 14 or 326 to 338, or a variant, fragment, derivative or fusion thereof, or fusions of said fragments, variants and derivatives thereof, which retain the activity specific to peptidoglycan,
   or wherein the first domain is encoded by a nucleotide sequence selected from any one of SEQ ID NOs: 1, 3, 5, 7, 9, 11 and 13, or 313 to 325.
8. The peptide according to any preceding paragraph, wherein the second domain is specific to the ester linkage between mycolic acid and arabinogalactan; or between mycolic acid and trehalose or between arabinogalactan and peptidoglycan.
9. The peptide according to any preceding paragraph, wherein the second domain is derived from alpha/beta hydrolases, such as esterases, lipases and/or cutinases.
10. The peptide according to any preceding paragraph, wherein the second domain is a mycolyl arabinogalactan esterase and/or a mycolyl-arabinogalactan-peptidoglycan (mAGP) hydrolase.
11. The peptide according to any preceding paragraph, wherein the second domain is a LysB and/or at least one portion thereof, optionally wherein the portion thereof is at least one enzymatically active domain (EAD).
12. The peptide according to any preceding paragraph, wherein the second domain comprises or consists of an amino sequence selected from any one of SEQ ID NOs: 16, 18, 20, 22, 24, 26, 28, 30 or 32, or a variant, fragment, derivative or fusion thereof, or fusions of said fragments, variants and derivatives thereof, which retain the activity specific to peptidoglycan,
   or wherein the first domain is encoded by a nucleotide sequence selected from any one of SEQ ID NOs: 15, 17, 19, 21, 23, 25, 27, 29 or 31.
13. The peptide according to any preceding paragraph, wherein the first domain is LysA or a portion thereof and/or the second domain is LysB or a portion thereof,
   optionally wherein the LysA and/or LysB comprise a cell wall binding domain (CBD).
14. The peptide according to any preceding paragraph, wherein the first domain is LysA or a portion thereof and comprises a cell wall binding domain (CBD) that is specific to the bacterial target of interest, optionally wherein the CBD is not specific to the cell walls of gram positive and/or gram negative bacteria, preferably wherein the CBD is specific to mycobacterial cell walls.
15. The peptide according to any preceding paragraph, wherein the peptide comprises multiple domains corresponding to a first domain, second domain, third domain, and/or fourth domain; optionally wherein the multiple domains are:
   (i) repeats of the same domain;
   (ii) different domains but with the same type of activity;
   (iii) derived from the same source (for example, all domains may be from the same organism);
   (iv) derived from different sources (for example, any one or more of the domains may be from a different organism to any other domain).
16. The peptide according to any preceding paragraph, wherein the third domain with membrane permeabilising, destabilizing and/or disrupting activity is an antimicrobial peptide (AMP) or portion thereof and/or a holin or portion thereof,
   optionally wherein the AMP or a portion thereof is cationic, polycationic, hydrophobic, amphipathic, synthetic or natural or any combination thereof (or comprises portions that are cationic, etc, or any combination thereof).
17. The peptide according to any preceding paragraph, wherein the second domain comprises or consists of an amino sequence selected from any one of SEQ ID NOs: 16, 18, 20, 22, 24, 26, 28, 30 or 32, or a variant, fragment, derivative or fusion thereof, or fusions of said fragments, variants and derivatives thereof, which retain the activity specific to peptidoglycan,
   or wherein the first domain is encoded by a nucleotide sequence selected from any one of SEQ ID NOs: 15, 17, 19, 21, 23, 25, 27, 29 or 31.
18. The peptide according to any preceding paragraph, wherein the domains are in the following order (N- to C-terminal):
   (a) at least one third domain—at least one second domain—at least one first domain;
   (b) at least one third domain—at least one first domain—at least one second domain;
   (c) at least one first domain—at least one second domain—at least one third domain;
   (d) at least one second domain—at least one first domain—at least one third domain;
   (e) at least one first domain—at least one third domain—at least one second domain;
   (f) at least one second domain—at least one third domain—at least one first domain;
      optionally wherein any of the peptides (or further, alternative configurations) further comprises at least one PTD and/or at least one linker, wherein the PTD and/or linker is at the N-terminus, C-terminus and/or between any of the domains.
19. The peptide according to any preceding paragraph, wherein the peptide further comprises at least one linker peptide, wherein the linker peptide connects:
   (i) the first domain to the second domain;
   (ii) the second domain to the third domain;
   (iii) the first domain to the third domain; and/or
   optionally wherein at least one linker comprises an amino acid sequence
   selected from SEQ ID NOs: 104, 106, 108, 110, 112 or 114;
   optionally wherein the length of the linker is between 1 amino acid and about 10 amino acids, and/or not in excess of 100 amino acids.
20. The peptide according to any preceding paragraph, wherein the first domain and/or second domain comprises a domain with a mechanism of action selected from the group consisting of amidase, transglycosylase, muramidase and/or peptidase, N-acetylmuramoyl-L-alanine amidase, Amidases in general including all families, D-Alanine-meso-Diaminopimelic (DD) endopeptidase, c-D-glutamyl-meso-diaminopimelic acid (DL) peptidase, Endopeptidases in general including all families, lytic transglycosylases, N-acetylmuramidase, lysozyme, Chitinase, L-alanoyl-D-glutamate (LD) endopeptidase, m-DAP-m-DAP (LD) endopeptidase in general, D-alanyl-D-alanine carboxypeptidase, Glycoside hydrolases in general including all families, L-Alanine-D-Glutamate peptidase, cysteine protease and N-acetyl-β-D-glucosaminidase, carboxypeptidase, glycosidase (glucosaminidases), transpeptidases, epimerase, Y-D-glutamyl-meso-diaminopimelic acid (DL) peptidases, and/or combinations thereof.
21. The peptide according to any preceding paragraph, wherein any one or more of the domains are effective against the phylum Actinobacteria; derived from the class Actinobacteridae; derived from the order Actinomycetales, and/or Bifidobacteriales. Optionally wherein at least one lysin is derived from a subclass and/or family selected from the list consisting of: Actinomycineae: Actinomycetaceae (Actinomyces, Mobiluncus); Corynebacterineae: Mycobacteriaceae (Mycobacterium), Nocardiaceae; Frankineae: Frankiaceae; Micrococcineae: Brevibacteriaceae; Propionibacteriaceae (Propionibacterium); Bifidobacteriaceae (Bifidobacterium, Falcivibrio, Gardnerella); Acidimicrobidae, Coriobacteridae, Rubrobacteridae, Sphaerobacteridae.
22. The peptide according to any preceding paragraph, wherein any one or more of the domains are effective against Mycobacterium selected from the group consisting of: Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abscessus, Mycobacterium kansasii, Mycobacterium terrae, Mycobacterium nonchromogenicum, Mycobacterium gordonae, and Mycobacterium triviale, and non-tuberculosis mycobacteria (NTM).
23. The peptide according to any preceding paragraph, wherein any one or more of the domains are derived from at least one mycobacteriophage, optionally wherein the mycobacteriophage is selected from the group consisting of: TM4, D29, L5, Bxz2, Saal, Enkosi, Ms6, Omega, Obama12, Echild, DS6A, Pumpkin or any other mycobacteriophage as listed in The Actinobacteriophage Database.
24. The peptide according to any preceding paragraph, wherein any one or more of the domains are a mutant, variant or wildtype domain.
25. The peptide according to any preceding paragraph, wherein at least one of the domains is a mutant or variant that comprises at least 10%, 20%, 30%, 40%, 50%, 60%, 70%, 80%, 90%, 95%, 96%, 97%, 98%, 99% or 100% of a wildtype sequence, optionally wherein the mutant or variant retains substantially the same level of activity of the wildtype domain.
26. A composition comprising a peptide as defined by any preceding paragraph, optionally wherein the composition comprises one or more further agents (e.g. pharmaceutically acceptable excipients).
27. The peptide according to any preceding paragraph for use in medicine, including veterinary medicine.
28. The peptide according to any preceding paragraph for use in targeting a cell envelope comprising:
   (i) an outer leaflet and inner leaflet;
   (ii) an arabinogalactan layer;
   (iii) a mycolic acid layer;
   (iv) a peptidoglycan layer;
   (v) a periplasmic space;
   (vi) a granular layer; and/or
   (vii) an inner membrane.
29. The peptide according to any preceding paragraph for use in:
   (i) promoting wound healing
   (ii) treating infection, optionally wherein the infection is mycobacterial infection or when the infection is caused by non-tuberculous mycobacteria (NTM)
   (iii) treating active tuberculosis and/or latent tuberculosis;
   (iv) treating respiratory, ocular, skin, oral and/or dental infections;
   (v) ocular, skin, oral and/or dental procedures;
   (vi) sterilisation (e.g. sterilisation via biocidal activity, wherein the peptide is acting as a biocide);
   (vii) treating granulomas; and/or
   (viii) treating biofilms.
30. The peptide, composition, and/or use of any preceding paragraph, wherein the bacterium (such as the mycobacterium) is resistant to at least one drug, optionally at least 2, 3, 4, 5, 6, 7, 8, 9, 10 or more drugs; and/or is classified as multidrug-resistant (MDR), extremely drug-resistant (XDR), or totally drug-resistant (TDR).
31. A polynucleotide comprising a nucleic acid encoding the peptide according to any preceding paragraph.
32. A vector comprising a peptide according to any preceding paragraph, and/or the nucleic acid according to paragraph 31.
33. A cell comprising a peptide according to any preceding paragraph, the nucleic acid according to paragraph 31, and/or the vector according to paragraph 32.
34. The peptide or composition for use of any preceding paragraph, wherein the peptide/composition is used in combination with at least one additional therapeutic agent, optionally wherein the additional therapeutic agent is one or more anti-mycobacterial agent, such as a mycobacterial chemotherapeutic.
35. An in vitro method of lysing, killing, reducing growth of, and/or reducing viability of mycobacteria, wherein the method comprises administering a peptide according to any preceding paragraph.
36. A (medical) device comprising a peptide according to any preceding paragraph.
37. A kit comprising an agent according to any preceding paragraph, optionally wherein the kit further comprises instructions for use.
38. A peptide, composition, use of a peptide or composition, a method of treatment or diagnosis, or a kit substantially as defined herein with reference to the description.

### FIGURE LEGENDS

FIG. 1: Schematic structure of mycobacterial cell envelope showing the target sites for Antimicrobial peptides (AMPs) acting on the inner and outer membranes, LysB enzymes hydrolyzing the ester bond that connects mycolic acid to the arabinogalactan-peptidoglycan and LysA enzymes that hydrolyze the peptidoglycan layer of the cell wall (Vincent et al., 2018).

FIG. 2: SDS-PAGE of some clones of the constructed library 1 expressed in LB medium, at 16° C. for 72 hr. L1: Molecular weight ladder (Allblue). L2: Clone 1A7 soluble fraction (MW: 53 kDa). L3: Clone 1A7 insoluble fraction (MW: 53 kDa). L4: Clone 1A11 Soluble fraction. L5: Clone 1A11 insoluble fraction (MW: 50 kDa). L6: Clone 1B4 Soluble fraction (MW: 95 kDa). L7: Clone 1B4 insoluble fraction (MW: 95 kDa). L8: Clone 1B6 Soluble fraction (MW: 50 kDa). L9: Clone 1B6 insoluble fraction (MW: 50 kDa). L10: Clone 1B7 soluble fraction (MW: 97 kDa). L11: Clone 1B7 Insoluble fraction (MW: 97 kDa). L12: Clone 1B7 LB medium supernatant (MW: 97 kDa). L13: Clone 1B8 Soluble fraction (MW: 100 kDa). L14: Clone 1B8 insoluble fraction (MW: 100 kDa). L15: Clone 1A11 LB medium supernatant (MW: 50 kDa).

FIG. 3: Optical density (OD_{625 nm}) of microtiter plate of Mycobacterium smegmatis 10⁸ CFU/ml after 24 hours incubation at 37° C. Fifty microliters of clarified cell lysate prepared from library 1 (AMP-LysB-Linker-LysA) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 4: Optical density (OD_{625 nm}) of microtiter plate of Mycobacterium smegmatis 10⁸ CFU/ml after 24 hours incubation at 37° C. Fifty microliters of clarified cell lysate prepared from library 2 (AMP-LysB-Linker-EAD) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 5: Optical density (OD_{625 nm}) of microtiter plate of Mycobacterium smegmatis 10⁸ CFU/ml after 24 hours incubation at 37° C. Fifty microliters of clarified cell lysate prepared from library 3 (AMP-LysB-Linker-LysA-PTD) were mixed with 50 μl of M. smegmatis with inoculum size of 10⁸ CFU/ml. A1-D10 (i.e. A1-A12, B1-B12, C1-C12 and D1-D10) represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 6: Optical density (OD_{625 nm}) of microtiter plate of Nocardia iowensis 10⁸ CFU/ml after 24 hours incubation at 37° C. Fifty microliters of clarified cell lysate prepared from library 1 (AMP-LysB-Linker-LysA) were mixed with 50 μl of Nocardia iowensis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=0.65).

FIG. 7: Optical density (OD_{625 nm}) of microtiter plate of Nocardia iowensis 10⁸ CFU/ml after 24 hours incubation at 37° C. Fifty microliters of clarified cell lysate prepared from library 2 (AMP-LysB-Linker-EAD) were mixed with 50 μl of Nocardia iowensis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=0.65).

FIG. 8: Optical density (OD_{625 nm}) of microtiter plate of Nocardia iowensis 10⁸ CFU/ml after 24 hours incubation at 37° C. Fifty microliters of clarified cell lysate prepared from library 3 (AMP-LysB-Linker-LysA-PTD) were mixed with 50 μl of Nocardia iowensis with inoculum size of 10⁸ CFU/ml. A1-D10 (i.e. A1-A12, B1-B12, C1-C12 and D1-D10) represents the positions of different constructs from the library within a 96-well plate. Wells with OD<0.1, indicating strong antibacterial activity, are marked with black colour and hit rate is the percentage of wells having OD<0.1. Positive control (OD_{625 nm}=0.65).

FIG. 9: Optical density (OD_{625 nm}) of microtiter plate of Rhodococcus erythropolis 108 CFU/ml after 24 hours incubation at 37° C. Fifty microliters of clarified cell lysate prepared from library 1 (AMP-LysB-Linker-LysA) were mixed with 50 μl of Rhodococcus erythropolis 108 with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=0.43).

FIG. 10: Optical density (OD_{625 nm}) of microtiter plate of Rhodococcus erythropolis 108 CFU/ml after 24 hours incubation at 37° C. Fifty microliters of clarified cell lysate prepared from library 2 (AMP-LysB-Linker-EAD) were mixed with 50 μl of Rhodococcus erythropolis 108 with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=0.43).

FIG. 11: Optical density (OD_{625 nm}) of microtiter plate of Rhodococcus erythropolis 108 CFU/ml after 24 hours incubation at 37° C. Fifty microliters of clarified cell lysate prepared from library 3 (AMP-LysB-Linker-LysA-PTD) were mixed with 50 μl of Rhodococcus erythropolis 108 with inoculum size of 10⁸ CFU/ml. A1-D10 (i.e. A1-A12, B1-B12, C1-C12 and D1-D10) represents the positions of different constructs from the library within a 96-well plate. Wells with OD<0.1, indicating strong antibacterial activity, are marked with black colour and hit rate is the percentage of wells having OD<0.1. Positive control (OD_{625 nm}=0.43).

FIG. 12: Percentage killing efficiency of 50 μl of clarified cell lysate prepared from library 1 (AMP-LysB-Linker-LysA) mixed with 50 μl of Mycobacterium bovis BCG strain with inoculum size of 106 CFU/ml after 24 hours incubation at 37° C. A1-H12 represents the positions of different constructs from the library within a 96-well plate.

FIG. 13: Percentage killing efficiency of 50 μl of clarified cell lysate prepared from library 2 (AMP-LysB-Linker-EAD) mixed with 50 μl of Mycobacterium bovis BCG strain with inoculum size of 106 CFU/ml after 24 hours incubation at 37° C. A1-H12 represents the positions of different constructs from the library within a 96-well plate.

FIG. 14: Percentage killing efficiency of 50 μl of clarified cell lysate prepared from library 3 (AMP-LysB-Linker-LysA-PTD) mixed with 50 μl of Mycobacterium bovis BCG strain with inoculum size of 106 CFU/ml after 24 hours incubation at 37° C. A1-D10 (i.e. A1-A12, B1-B12, C1-C12 and D1-D10) represents the positions of different constructs from the library within a 96-well plate.

FIG. 15: Percentage killing efficiency of 50 μl of clarified cell lysate prepared from library 1 (AMP-LysB-Linker-LysA) mixed with 50 μl of Mycobacterium abscessus with inoculum size of 10⁷ CFU/ml after 24 hours incubation at 37° C. A1-H12 represents the positions of different constructs from the library within a 96-well plate.

FIG. 16: Percentage killing efficiency of 50 μl of clarified cell lysate prepared from library 2 (AMP-LysB-Linker-EAD) mixed with 50 μl of Mycobacterium abscessus with inoculum size of 10⁷ CFU/ml after 24 hours incubation at 37° C. A1-H12 represents the positions of different constructs from the library within a 96-well plate.

FIG. 17: Percentage killing efficiency of 50 μl of clarified cell lysate prepared from library 3 (AMP-LysB-Linker-LysA-PTD) mixed with 50 μl of Mycobacterium abscessus with inoculum size of 10⁷ CFU/ml after 24 hours incubation at 37° C. A1-D10 (i.e. A1-A12, B1-B12, C1-C12 and D1-D10) represents the positions of different constructs from the library within a 96-well plate.

FIG. 18: Optical density (OD_{625 nm}) of microtiter plate of Mycobacterium smegmatis 10⁸ CFU/ml after 24 hours incubation at 37° C. Fifty microliters of clarified cell lysate prepared from library 4 (AMP-LysB) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 19: Optical density (OD_{625 nm}) of microtiter plate of Mycobacterium smegmatis 10⁸ CFU/ml after 24 hours incubation at 37° C. Fifty microliters of clarified cell lysate prepared from library 5 (AMP-Linker-LysB) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 20: Optical density (OD_{625 nm}) of microtiter plate of Mycobacterium smegmatis 10⁸ CFU/ml after 24 hours incubation at 37° C. Fifty microliters of clarified cell lysate prepared from library 6 (AMP-LysA) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 21: Optical density (OD_{625 nm}) of microtiter plate of Mycobacterium smegmatis 10⁸ CFU/ml after 24 hours incubation at 37° C. Fifty microliters of clarified cell lysate prepared from library 7 (AMP-Linker-LysA) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 22: Optical density (OD_{625 nm}) of microtiter plate of Mycobacterium smegmatis 10⁸ CFU/ml after 24 hours incubation at 37° C. Fifty microliters of clarified cell lysate prepared from library 8 (AMP-EAD) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 23: Optical density (OD_{625 nm}) of microtiter plate of Mycobacterium smegmatis 10⁸ CFU/ml after 24 hours incubation at 37° C. Fifty microliters of clarified cell lysate prepared from library 9 (AMP-Linker-EAD) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 24: Optical density (OD_{625 nm}) of microtiter plate of Mycobacterium smegmatis 10⁵ CFU/ml after 24 hours incubation at 37° C. Fifty microliters of clarified cell lysate prepared from library 10 (LysB-Linker-LysA) were mixed with 50 μl of M. smegmatis with inoculum size of 10⁵ CFU/ml. A1-H12 represents the positions of different constructs from the library within a 96-well plate. Wells with OD<0.1, indicating strong antibacterial activity, are marked with black colour and hit rate is the percentage of wells having OD<0.1.

FIG. 25: Optical density (OD_{625 nm}) of microtiter plate of Mycobacterium smegmatis 10⁵ CFU/ml after 24 hours incubation at 37° C. Fifty microliters of clarified cell lysate prepared from library 11 (LysB-Linker-EADs) were mixed with 50 μl of M. smegmatis with inoculum size of 10⁵ CFU/ml. A1-H12 represents the positions of different constructs from the library within a 96-well plate. Wells with OD<0.1, indicating strong antibacterial activity, are marked with black colour and hit rate is the percentage of wells having OD<0.1.

FIG. 26: Optical density (OD_{625 nm}) of 50 μl Mycobacterium smegmatis 10⁵ CFU/ml mixed with 50 μl of clarified cell lysate prepared from LysB enzymes after 24 hours incubation at 37° C. Fifty microliters of Mueller-Hinton broth mixed with 50 μl M. smegmatis cells 10⁵ CFU/ml was considered as positive control (OD=0.4). Wells with OD<0.1, indicating strong antibacterial activity, are marked with black colour and hit rate is the percentage of wells having OD<0.1.

FIG. 27: Optical density (OD_{625 nm}) of 50 μl Mycobacterium smegmatis 10⁵ CFU/ml mixed with 50 μl of clarified cell lysate prepared from LysA enzymes after 24 hours incubation at 37° C. Fifty microliters of Mueller-Hinton broth mixed with 50 μl M. smegmatis cells 10⁵ CFU/ml was considered as positive control (OD=0.4). Wells with OD<0.1, indicating strong antibacterial activity, are marked with black colour and hit rate is the percentage of wells having OD<0.1.

FIG. 28: Optical density (OD_{625 nm}) of 50 μl Mycobacterium smegmatis 10⁵ CFU/ml mixed with 50 μl of clarified cell lysate prepared from some selected Enzyme Active Domains (EADs) after 24 hours incubation at 37° C. Fifty microliters of Mueller-Hinton broth mixed with 50 μl M. smegmatis cells 10⁵ CFU/ml was considered as positive control (OD=0.4). Wells with OD<0.1, indicating strong antibacterial activity, are marked with black colour and hit rate is the percentage of wells having OD<0.1.

FIG. 29: Optical density (OD_{625 nm}) of microtiter plate of E. coli 10⁸ CFU/ml after 24 hours incubation at 37° C. Fifty microliters of clarified cell lysate prepared from library 1 (AMP-LysB-Linker-LysA) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 30: Optical density (OD_{625 nm}) of microtiter plate of E. coli 10⁸ CFU/ml after 24 hours incubation at 37° C. Fifty microliters of clarified cell lysate prepared from library 2 (AMP-LysB-Linker-EAD) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 31: Optical density (OD_{625 nm}) of microtiter plate of E. coli 10⁸ CFU/ml after 24 hours incubation at 37° C. Fifty microliters of clarified cell lysate prepared from library 3 (AMP-LysB-Linker-LysA-PTD) were mixed with 50 μl of E. coli with inoculum size of 10⁸ CFU/ml. A1-D10 (i.e. A1-A12, B1-B12, C1-C12 and D1-D10) represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 32: Optical density (OD_{625 nm}) of microtiter plate of Mycobacterium smegmatis 10⁸ CFU/ml after 24 hours incubation at 37° C. Fifty microliters of clarified cell lysate prepared from library 12 (AMP-LysA-Linker-LysB) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 33: Optical density (OD_{625 nm}) of microtiter plate of E. coli 10⁸ CFU/ml after 24 hours incubation at 37° C. Fifty microliters of clarified cell lysate prepared from library 12 (AMP-LysA-Linker-LysB) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 34: Optical density (OD_{625 nm}) of microtiter plate of Mycobacterium smegmatis 10⁸ CFU/ml after 24 hours incubation at 37° C. Fifty microliters of clarified cell lysate prepared from library 13 (AMP-EAD-Linker-LysB) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 35: Optical density (OD_{625 nm}) of microtiter plate of E. coli 10⁸ CFU/ml after 24 hours incubation at 37° C. Fifty microliters of clarified cell lysate prepared from library 13 (AMP-EAD-Linker-LysB) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 36: Optical density (OD_{625 nm}) of microtiter plate of Mycobacterium smegmatis 10⁸ CFU/ml after 24 hours incubation at 37° C. Fifty microliters of clarified cell lysate prepared from library 14 (LysB-Linker-LysA-AMP) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 37: Optical density (OD_{625 nm}) of microtiter plate of E. coli 10⁸ CFU/ml after 24 hours incubation at 37° C. Fifty microliters of clarified cell lysate prepared from library 14 (LysB-Linker-LysA-AMP) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 38: Optical density (OD_{625 nm}) of microtiter plate of Mycobacterium smegmatis 10⁸ CFU/ml after 24 hours incubation at 37° C. Fifty microliters of clarified cell lysate prepared from library 15 (LysA-Linker-LysB-AMP) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 39: Optical density (OD_{625 nm}) of microtiter plate of E. coli 10⁸ CFU/ml after 24 hours incubation at 37° C. Fifty microliters of clarified cell lysate prepared from library 15 (LysA-Linker-LysB-AMP) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 40: Optical density (OD_{625 nm}) of microtiter plate of Mycobacterium smegmatis 10⁸ CFU/ml after 24 hours incubation at 37° C. Fifty microliters of clarified cell lysate prepared from library 16 (LysB-AMP-LysA) were mixed with 50 μl of M. smegmatis with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.3).

FIG. 41: Optical density (OD_{625 nm}) of microtiter plate of E. coli 10⁸ CFU/ml after 24 hours incubation at 37° C. Fifty microliters of clarified cell lysate prepared from library 16 (LysB-AMP-LysA) were mixed with 50 μl of E. coli with inoculum size of 10⁸ CFU/ml. A1-H12 represents the positions of different constructs from the library within a 96-well plate. Wells with OD<0.1, indicating strong antibacterial activity, are marked with black colour and hit rate is the percentage of wells having OD<0.1. Positive control (OD_{625 nm}=1.4).

FIG. 42: A comparison of the growth curve based on optical density (OD_{625 nm}) for various clones (AMP-LysB, AMP-LysA and 4D10) in LB medium. AMP=Ci-MAM-A24, LysB=D29, Linker=Linker 1, and LysA=TM4. 4D10 includes each component, culminating in [Ci-MAM-A24]-[D29]-[Linker]-[TM4]. Optical density positively correlates with expression levels, in that a higher optical density means a higher level of expression of the peptide. 4D10 shows considerably faster expression compared with both AMP-LysB and AMP-LysA.

FIG. 43: A comparison of the antimycobacterial activity against M. smegmatis for LysA alone; LysB alone; a mixture of LysA and LysB; AMP-LysA alone; AMP-LysB alone; a mixture of AMP-LysA and AMP-LysB; and AMP-LysB-Linker-LysA alone.

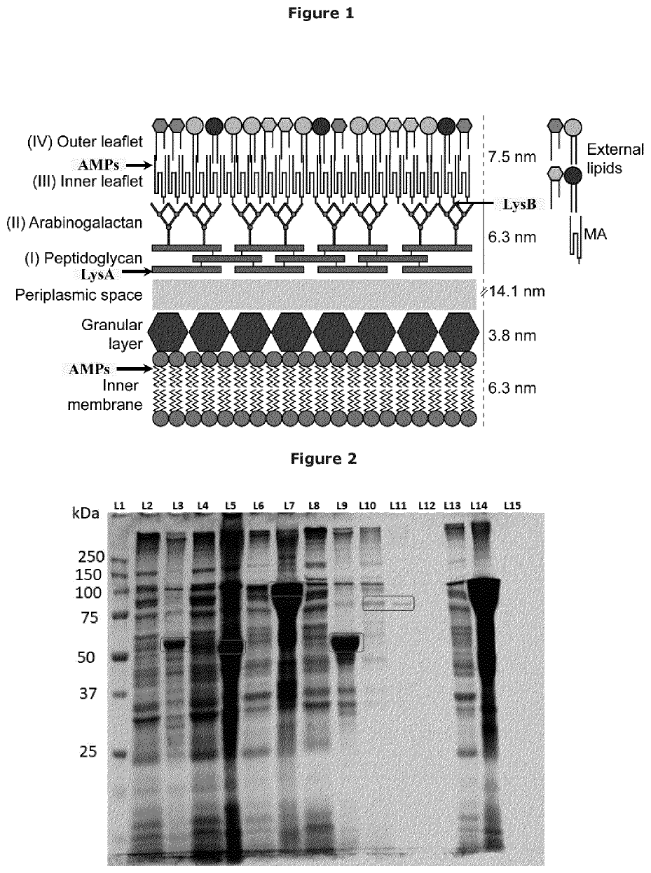

### REFERENCES

1. Bhamidi, S., Scherman, M. S., Rithner, C. D., Prenni, J. E., Chatterjee, D., Khoo, K. H., McNeil, M. R. 2008. The identification and location of succinyl residues and the characterization of the interior arabinan region allow for a model of the complete primary structure of Mycobacterium tuberculosis mycolyl arabinogalactan. Journal of Biological Chemistry, 283 (19), 12992-13000.
2. Brennan, P. J. 2003. Structure, function, and biogenesis of the cell wall of Mycobacterium tuberculosis. Tuberculosis, 83 (1-3), 91-97.
3. Brennan, P. J., Nikaido, H. 1995. The Envelope of Mycobacteria. Annual Review of Biochemistry, 64, 29-63.
4. Briers, Y., Walmagh, M., Van Puyenbroeck, V., Cornelissen, A., Cenens, W., Aertsen, A., Oliveira, H., Azeredo, J., Verween, G., Pirnay, J. P., Miller, S., Volckaert, G., Lavigne, R. 2014. Engineered Endolysin-Based “Artilysins” To Combat Multidrug-Resistant Gram-Negative Pathogens. Mbio, 5 (4).
5. Cobb, R. E., Chao, R., Zhao, H. M. 2013. Directed Evolution: Past, Present, and Future. Aiche Journal, 59 (5), 1432-1440.
6. Crameri, A., Raillard, S. A., Bermudez, E., Stemmer, W. P. C. 1998. DNA shuffling of a family of genes from diverse species accelerates directed evolution. Nature, 391 (6664), 288-291.
7. Daffe, M. 2008. The Global Architecture of the Mycobacterial Cell Envelope. Mycobacterial Cell Envelope, 3-11.
8. Daffe, M. 1996. Structure of the cell envelope of Mycobacterium tuberculosis. Medecine Et Maladies Infectieuses, 26 (11), 891-897.
9. Engler, C., Gruetzner, R., Kandzia, R., Marillonnet, S. 2009. Golden Gate Shuffling: A One-Pot DNA Shuffling Method Based on Type IIs Restriction Enzymes. Plos One, 4 (5).
10. Engler, C., Kandzia, R., Marillonnet, S. 2008. A One Pot, One Step, Precision Cloning Method with High Throughput Capability. Plos One, 3 (11).
11. Gerstmans, H., Criel, B., Briers, Y. 2018. Synthetic biology of modular endolysins. Biotechnology Advances, 36 (3), 624-640.
12. Grimon, D., Gerstmans, H., Briers, Y., Lavigne, R. 2019. Polynucleotide shuffling method, US 2019/0323017 A1.
13. Harayama, S. 1998. Artificial evolution by DNA shuffling. Trends in Biotechnology, 16 (2), 76-82.
14. Hoffmann, C., Leis, A., Niederweis, M., Plitzko, J. M., Engelhardt, H. 2008. Disclosure of the mycobacterial outer membrane: Cryo-electron tomography and vitreous sections reveal the lipid bilayer structure. Proceedings of the National Academy of Sciences of the United States of America, 105 (10), 3963-3967.
15. Jarlier, V., Nikaido, H. 1990. Permeability barrier to hydrophilic solutes in Mycobacterium chelonei. J Bacteriol, 172 (3), 1418-23.
16. Lane, M. D., Seelig, B. 2014. Advances in the directed evolution of proteins. Current Opinion in Chemical Biology, 22, 129-136.
17. Marrakchi, H., Laneelle, M. A., Daffe, M. 2014. Mycolic acids: structures, biosynthesis, and beyond. Chem Biol, 21 (1), 67-85.
18. Mcneil, M., Daffe, M., Brennan, P. J. 1990. Evidence for the Nature of the Link between the Arabinogalactan and Peptidoglycan of Mycobacterial Cell-Walls. Journal of Biological Chemistry, 265 (30), 18200-18206.
19. Miller, S. 2015. Composition for use in mycobacteria therapy, Google Patents.
20. Mitnick, C. D., McGee, B., Peloquin, C. A. 2009. Tuberculosis pharmacotherapy: strategies to optimize patient care. Expert Opin Pharmacother, 10 (3), 381-401.
21. Muller, B., Durr, S., Alonso, S., Hattendorf, J., Laisse, C. J. M., Parsons, S. D. C., van Helden, P. D., Zinsstag, J. 2013. Zoonotic Mycobacterium bovis-induced Tuberculosis in Humans. Emerging Infectious Diseases, 19 (6), 899-908.
22. Nelson, D. C., Schmelcher, M., Rodriguez-Rubio, L., Klumpp, J., Pritchard, D. G., Dong, S. L., Donovan, D. M. 2012. Endolysins as Antimicrobials. Advances in Virus Research, Vol 83: Bacteriophages, Pt B, 83, 299-365.
23. Neyrolles, O., Guilhot, C. 2011. Recent advances in deciphering the contribution of Mycobacterium tuberculosis lipids to pathogenesis. Tuberculosis (Edinb), 91 (3), 187-95.
24. Niederweis, M. 2008. Nutrient acquisition by mycobacteria. Microbiology-Sgm, 154, 679-692.
25. Nordwald, E. M., Garst, A., Gill, R. T., Kaar, J. L. 2013. Accelerated protein engineering for chemical biotechnology via homologous recombination. Current Opinion in Biotechnology, 24 (6), 1017-1022.
26. Pritchard, L., Corne, D., Kell, D., Rowland, J., Winson, M. 2005. A general model of error-prone PCR. Journal of Theoretical Biology, 234 (4), 497-509.
27. Sani, M., Houben, E. N. G., Geurtsen, J., Pierson, J., de Punder, K., van Zon, M., Wever, B., Piersma, S. R., Jimenez, C. R., Daffe, M., Appelmelk, B. J., Bitter, W., van der Wel, N., Peters, P. J. 2010. Direct Visualization by Cryo-EM of the Mycobacterial Capsular Layer: A Labile Structure Containing ESX-1-Secreted Proteins. Plos Pathogens, 6 (3).
28. Schmelcher, M., Donovan, D. M., Loessner, M. J. 2012. Bacteriophage endolysins as novel antimicrobials. Future Microbiology, 7 (10), 1147-1171.
29. Sharma, U. K. 2017. Phage-derived compositions for improved mycobacterial therapy, Google Patents.
30. Tee, K. L., Wong, T. S. 2013. Polishing the craft of genetic diversity creation in directed evolution. Biotechnology Advances, 31 (8), 1707-1721.
31. Tenland, E., Krishnan, N., Ronnholin, A., Kalsum, S., Puthia, M., Morgelin, M., Davoudi, M., Otrocka, M., Alaridah, N., Glegola-Madejska, I., Sturegard, E., Schmidtchen, A., Lerm, M., Robertson, B. D., Godaly, G. 2018. A novel derivative of the fungal antimicrobial peptide plectasin is active against Mycobacterium tuberculosis. Tuberculosis, 113, 231-238.
32. Vilcheze, C., Jacobs, W. R. 2007. The mechanism of isoniazid killing: Clarity through the scope of genetics. Annual Review of Microbiology, 61, 35-50.
33. Vincent, A. T., Nyongesa, S., Morneau, I., Reed, M. B., Tocheva, E. I., Veyrier, F. J. 2018. The Mycobacterial Cell Envelope: A Relict From the Past or the Result o Recent Evolution? Frontiers in Microbiology, 9.
34. Watanabe, M., Aoyagi, Y., Ridell, M., Minnikin, D. E. 2001. Separation and characterization of individual mycolic acids in representative mycobacteria. Microbiology-Sgm, 147, 1825-1837.
35. Zuber, B., Chami, M., Houssin, C., Dubochet, J., Griffiths, G., Daffe, M. 2008. Direct visualization of the outer membrane of mycobacteria and corynebacteria in their native state. Journal of Bacteriology, 190 (16), 5672-5680.
36. Abouhmad, A., Korany, A. H., Grey, C., Dishisha, T., Hatti-Kaul, R. 2020. Exploring the Enzymatic and Antibacterial Activities of Novel Mycobacteriophage Lysin B Enzymes. International Journal of Molecular Sciences, 21 (3176).
37. Velayati A. A, Farnia P, Ibrahim T. A, Haroun R. Z, Kuan H. O, Ghanavi J, Farnia P, Kabarei A. N, Tabarsi P, Omar A. R, Varahram M, Masjedi M. R. 2009. Differences in Cell Wall Thickness between Resistant and Nonresistant Strains of Mycobacterium tuberculosis: Using Transmission Electron Microscopy. Chemotherapy, 55:303-307
38. Kongari, R., Rajaure, M., Cahill, J., Rasche, E., Mijalis, E., Berry, J., Young, R. 2018. Phage spanins: diversity, topological dynamics and gene convergence. BMC Bioinformatics, 19 (326).
39. Gutiérrez Fernández, D., Briers, Y. 2020. Developments and opportunities of bacteriophage lytic proteins for therapeutics against gram-negative pathogens. Semantic Scholar, DOI: 10.21775/9781913652517.15, Corpus ID: 219418090.
40. Gerstmans, H, Grimon, D., Gutiérrez, D., Lood, C., Rodríguez, A., vanNoort, V., Lammertyn, J., Lavigne, R., Briers, Y. 2020. A VersaTile-driven platform for rapid hit-to-lead development of engineered lysins. Science Advances, 6 (23).
41. van Schie L, Borgers K, Michielsen G, Plets E, Vuylsteke M, Tiels P, Festjens N, Callewaert N. Exploration of Synergistic Action of Cell Wall-Degrading Enzymes against Mycobacterium tuberculosis. Antimicrob Agents Chemother. 2021 Sep. 17; 65 (10): e0065921. doi: 10.1128/AAC.00659-21. Epub 2021 Jul. 19. PMID: 34280017; PMCID: PMC8448103.

### EXAMPLES

### Example 1

### Materials and Methods

Different LysB and LysA genes were codon optimized and synthesized as gene fragments (gBlock) (FIG. 2) from Integrated DNA Technologies (IDT, Leuven, Belgium). The gene fragments were amplified with PCR using Phusion® High fidelity DNA polymerase (Thermo Scientific®, Pittsburgh, PA, USA) to include the restriction site of the BsaI restriction enzyme using the following PCR primers (SEQ ID NOs: 115-146, see Table 1) and cloned into pVTSEIII cloning vector with the aid of BsaI restriction enzyme and transformed into E. coli TOP 10 cloning host (Grimon et al., 2019).

TABLE 1
PCR primers.

| Target | Forward Primer (5′-3′) | Reverse Primer (5′-3′) |
|---|---|---|
| LysB_D29 | GCAGCTCTTC AAGAGGTCTC AGTGCAAGCA | CTTGCTCTTCGCTTGGTCTC GCCTGCGATC |
| | AGCCCTGGCT G | TGTCGTAGGA ACTCGACCGC |
| | (SEQ ID NO: 115) | (SEQ ID NO: 116) |
| LysB_Omega | GTAGCTCTTC CAGAGGTCTC GGTGCTTTGA | GCAGCTCTTCGCTTGGTCTCGCCTGCATTT |
| | ACGGTGAATT TTACGTTCCG G | TTACGAAGTT CATTGCCCGT GC |
| | (SEQ ID NO: 117) | (SEQ ID NO: 118) |
| LysB_Saal | TCAGCTCTTC CAGAGGTCTC GGTGCTCGCA | CGAGCTCTTC CCTTGGTCTC |
| | TCGACGGTCA ATATGTG | GCCTGCTGTG CGCAGGTAAT CGATCG |
| | (SEQ ID NO: 119) | (SEQ ID NO: 120) |
| LysB_DS6A | GTCGCTCTTC CAGAGGTCTC GGTGCTACCT | ATTGCTCTTC GCTTGGTCTC |
| | GGATTGGCTG GC | GCCTGCCGCG GCTAAAGCCA AGC |
| | (SEQ ID NO: 121) | (SEQ ID NO: 122) |
| LysB_ | ATGGCTCTTC TAGAGGTCTC GGTGCTCTGA | TGTGCTCTTC ACTTGGTCTC |
| Obama12 | AGTTAGGATC AAACGGGC | GCCTGCTGTT ACAGACGCGA CAATGCG |
| | (SEQ ID NO: 123) | (SEQ ID NO: 124) |
| LysB_Enkosi | GCTGCTCTTC TAGAGGTCTC GGTGCTTCTA | GTTGCTCTTC GCTTGGTCTC |
| | AGCCTGTGCT GTTGACTGC | ACCTGCGGCG GCCATAGCGC G |
| | (SEQ ID NO: 125) | (SEQ ID NO: 126) |
| LysB_Bxz2 | TCGGCTCTTC TAGAGGTCTC GGTGCTCCGT | AGTGCTCTTCACTTGGTCTCGCCTGCTGAG |
| | TACGCGTGGG C | CGAAGAAAAT CAATCGCCG |
| | (SEQ ID NO: 127) | (SEQ ID NO: 128) |
| LysB_L5 | CGTGCTCTTCTAGAGGTCTC TGTGCTAGTA | GCCGCTCTTC GCTTGGTCTC |
| | AACCGTGGTT ATTTACTGTG C | GCCTGCAATG CGACGCAGAA ATTCTACG |
| | (SEQ ID NO: 129) | (SEQ ID NO: 130) |
| LysB_Ms6 | CTCGCTCTTCTAGAGGTCTC GGTGCTCGTA | ATCGCTCTTC GCTTGGTCTC |
| | TCGACGGCCA ATATGTTGG | GCCTGCCGTG CGGAGATAGT CG |
| | (SEQ ID NO: 131) | (SEQ ID NO: 132) |
| LysA_DS6A | TATGCTCTTCTAGAGGTCTC | CTAGCTCTTC GCTTGGTCTC |
| | GGAAGCATGTTAACTGTAGAGAGCTTCGCTGC | TGAGCCACGA CCAGCTTCGC GATCTGC |
| | (SEQ ID NO: 133) | (SEQ ID NO: 134) |
| LysA_BTCU | GTAGCTCTTC TAGAGGTCTC AGAAGCACAG | GTTGCTCTTCGCTTGGTCTC TGAGCCTTGT |
| | AGCGTGTCCT GC | TCAATAATGC GATTATGTGT AAC |
| | (SEQ ID NO: 135) | (SEQ ID NO: 136) |
| LysA_Bxb1 | TCAGCTCTTC TAGAGGTCTC GGAAGCCCTC | CGAGCTCTTC GCTTGGTCTC |
| | GCGTGGTCTA TGGTC | TGAGCCCGCC GCGTGAAATT TCTG |
| | (SEQ ID NO: 137) | (SEQ ID NO: 138) |
| LysA_D29 | GTCGCTCTTC TAGAGGTCTC GGAAGCACCT | TGTGCTCTTC GCTTGGTCTC |
| | TGATTGTAAC GCGCG | TGAGCCTAAC GCGCCGTTGC G |
| | (SEQ ID NO: 139) | (SEQ ID NO: 140) |
| LysA_Giles | ATCGCTCTTC TAGAGGTCTC GGAAGCCCTG | TGTGCTCTTC GCTTGGTCTC |
| | TCTATGCGAT TCAGTCTG | TGAGCCCAAA ACCAGGCCGA TTTTGG |
| | (SEQ ID NO: 141) | (SEQ ID NO: 142) |
| LysA_Ms6 | ACTGCTCTTC TAGAGGTCTC GGAAGCACTA | TGCGCTCTTC GCTTGGTCTC |
| | CAAAGGACCA GGTAGCG | TGAGCCATGT CCCAGCAACT CCG |
| | (SEQ ID NO: 143) | (SEQ ID NO: 144) |
| LysA_TM4 | GCTGCTCTTC TAGAGGTCTC GGAAGCTCAT | GTAGCTCTTC GCTTGGTCTC |
| | TTACGCGCTT TCTCCAAG | TGAGCCTGCC GCGCCACCAT C |
| | (SEQ ID NO: 145) | (SEQ ID NO: 146) |

The transformation mixtures were plated on LB agar plates supplemented with ampicillin (100 μg/ml) and 5% sucrose as selection markers. Plasmids were extracted from colonies 5 and were verified by sequencing (LGC genomics GmbH, Berlin, Germany). The clones with the right sequences were used as tiles for library construction and further in DNA shuffling reactions. Glycerol stocks of the tiles with the correct sequences cloned in pVTSEIII and transformed into E. coli Top 10 were prepared and stored at −80° C.

### Construction of Libraries

To construct a library for DNA shuffling, different tiles comprising different sets of enzymes were assembled together according to their required positions into pVTSDI, II and III expression vectors with the aid of SapI restriction enzyme (Grimon et al., 2019).

The positions in the final assembly of the DNA shuffling reactions were as follows:
For library 1: N-terminal 6×Histag from pVTSDI expression vector followed by different variants of antimicrobial peptides (AMPs) with different properties (cationic, polycationic, hydrophobic or amphipathic) are in position 1, LysB enzyme variants in position 2, linkers with different properties (flexible or rigid, helix or coil, short or long) are in position 3 and finally LysA enzyme variants with different mechanisms of actions are in position 4.
For library 2: antimicrobial peptides (AMPs) variants with different properties (cationic, polycationic, hydrophobic or amphipathic) are in position 1, LysB enzyme variants in position 2, linkers with different properties (flexible or rigid) are in position 3 and finally different variants of enzyme active domains (EADs) with different mechanisms of actions are in position 4 followed by 6×Histag from the pVTSDIII expression vector.
For library 3: variants of antimicrobial peptides (AMPs) with different properties (cationic, polycationic, hydrophobic or amphipathic) are in position 1, LysB enzymes in position 2, linkers with different properties (flexible or rigid) are in position 3 and finally LysA enzymes with different mechanisms of actions are in position 4 and finally protein transduction domains (PTDs) in position 5 followed by 6×Histag from the pVTSDIII expression vector. Moreover, different libraries were constructed for exclusion criteria, through removing one of the modules, the libraries are summarized in Table 2. The libraries were constructed with the aid of SapI restriction enzyme and T4 DNA ligase enzyme with the following cycling parameters (Table 3) in a thermal cycler.

TABLE 2
Organization and positions of the modules in the designed libraries.

| Library | Pos1 | Pos2 | Pos3 | Pos4 | Pos5 |
|---|---|---|---|---|---|
| 1 | AMPs | LysB | Linker | LysA | — |
| 2 | AMPs | LysB | Linker | EADs | |
| 3 | AMPs | LysB | Linker | LysA | PTDs |
| 4 | AMPs | LysB | — | — | — |
| 5 | AMPs | Linker | LysB | — | — |
| 6 | AMPs | LysA | — | — | |
| 7 | AMPs | Linker | LysA | — | — |
| 8 | AMPs | EADs | — | — | — |
| 9 | AMPs | Linker | EADs | — | — |
| 10 | LysB | Linker | LysA | — | — |
| 11 | LysB | Linker | EADs | — | — |

Swapping the positions of any of the components in Table 2 would not be expected to impact the functionality of the peptides derived from said libraries. For example, swapping LysA and LysB in libraries 1-3 would be expected to yield peptides with similar activities as those derived from libraries 1-3. Likewise, the AMP may be N-terminal, C-terminal or between other domains, without negatively impacting the overall activity of peptides derived from such libraries. Further, increasing the number of modules (e.g. having AMP at both termini) may be expected to enhance activity further.

TABLE 3
Thermal cycler parameters used for the shuffling reactions
(Grimon et al., 2019, US 2019/0323017 A1).

| | Temperature | | Number |
|---|---|---|---|
| Step | (° C.) | Time | of cycles |
| Restriction | 37° C. | 2 min | 30 |
| Ligation | 16° C. | 3 min | |
| Ligase inactivation | 50° C. | 5 min | 1 |
| Type IIs inactivation | 65° C. | 20 min | 1 |

### Expression of Libraries

The ligation mixture comprising different libraries was transformed into chemically competent E. coli codon plus expression host according to the manufacturer's instructions and plated on LB agar plates supplemented with kanamycin (50 μg/ml) and 5% sucrose as selection markers and incubated at 37° C. for overnight.

For preculture preparation, colonies were picked up with sterile toothpicks and used to inoculate 200 μl of LB medium in 96 well sterile plates supplemented with kanamycin (50 μg/ml) and 5% sucrose as selection markers and incubated at 37° C. for overnight, 100 μl of the 96 well preculture plates were used to prepare glycerol stocks and stored at −20° C.

Each well contains a different clone, i.e. the 96 well plate is representing 96 different clones. For expression of libraries, 20 μl of the precultures were added to 500 μl of autoinduction medium (containing per liter: 10 g α-lactose, 2.5 g glucose, 5 g glycerol, 2 mM KH₂PO₄, 2 mM MgSO₄, 50 mM Na₂HPO₄, 25 mM (NH₄)₂SO₄, 5 g yeast extract and 10 g of tryptone) in 96 deep well plates. The deep well plates were incubated at 37° C., 1000 rpm (IKA-Vibramax-VXR; IKA-Labortechnik, Staufen, Germany) for the first 4 hours and then continued for 48 hours at 30° C. The cells were harvested by centrifugation (4500 rpm, 4° C., 20 min, Sigma 3-16PK), the supernatant was discarded, and the cell pellet was used for cell lysate preparation. E. coli codon plus expression strain transformed with blank plasmids (plasmids without any inserts) were subjected to the same cultivation and expression conditions and used as controls.

The clarified cell lysate was loaded on SDS-PAGE to check the expression levels of the induced clones. For expression, LB medium was applied. The inocula was prepared by inoculating 10 ml LB medium supplied with kanamycin (50 μg/ml) with 50 μl glycerol stock of the corresponding clones, incubated overnight at 37° C., 200 rpm. The inocula were used to inoculate 100 ml of LB medium supplemented with kanamycin (50 μg/ml) and incubated at 37° C., 200 rpm till the OD600ₙₘ of ≈0.5-0.6. For induction, IPTG was added to a final concentration of 1 mM and the cultivation was continued at 30° C. for extra 4 hours.

To test the effect of temperature on the expression levels and the solubility of the induced proteins, induction with IPTG (1 mM) was performed at 16° C., 180 rpm for 72 hours. To check the expression levels and solubility of the expressed proteins, samples were taken ate the end of the induction period and checked by SDS-PAGE.

### Preparation of Cell Lysate

The cells were lysed by exposure to chloroform vapours, the deep-well plates were put upside-down in a glass chamber containing filter papers on its bottom. Twenty milliliters of chloroform were added, then immediately the glass chamber was sealed, and the deep well plates were incubated above a chloroform-saturated filter papers for 2 hours in the fume hood.

Later on, the plates were inverted and set to stand for another 15 minutes in the fume hood, the cell pellet was resuspended in 500 μl of resuspension buffer (50 mM Tris-HCL, 50 mM NaCl, pH 8) supplemented with 1 U of DNase I enzyme and incubated at 30° C., 100 rpm for 1 hour. The cell debris was removed by centrifugation (4500 rpm, 4° C., 60 min, Sigma 3-16PK) and the clarified cell lysate was stored at 4° C. for further screening. LB cultures were lysed using BugBuster® (Novagen, Madison, WI, USA) cell lysis solution according to the manufacturer's instructions. The lysed cell suspension was centrifuged at 14000 rpm, 30 min at 20° C. The crude extract as well as the cell debris (after resuspension in the same starting volume in 50 mM Tris, 50 mM NaCl, pH 8) were loaded to SDS-PAGE.

### Screening of the Antibacterial Activity of the Expressed Libraries

Screening of the antibacterial activity was done through antibacterial (growth) assay method. Any suitable screening method can be used to determine activity of the library, and screening methods are known to the skilled person, such as the screening method used in Gerstmans et al., 2020 and Tenland et al., 2018 and Van Schie et al. 2021.

Briefly, the inocula of the test strains (Mycobacterium smegmatis mc² 155; ATCC 700084, Mycobacterium abscessus, Nocardia iowensis, Rhodococcus erythropolis and Staphylococcus aureus) were grown overnight in Mueller-Hinton broth (MH) broth at 37° C., 200 rpm. For initial screening of the antibacterial activity, the final inoculum size (bacterial load) of the M. smegmatis in the assay was adjusted to (≈4×10⁵ CFU/ml) through dilution in 2×MHB. To increase the stringency of the assay conditions the final inoculum size of M. smegmatis in the assay was adjusted (≈4×10⁷ CFU/ml) and (≈4×10⁸ CFU/ml). For the assay, 50 μl of different inoculum size was mixed with 50 μl of the clarified cell lysate in a microtiter plate and incubated at 37° C. for 24 h.

Endpoint measurement at OD625 nm was performed in a microplate reader (Multiskan™ GO Microplate Spectrophotometer, Thermo Scientific) after 24 h and the MIC can be determined by eye as well. For determination of the bactericidal effect, 50 μl of the clear wells (wells with no growth) was spotted in LB agar and incubated for 24 h at 37° C. and examined for growth. A well with 100 μl 2×MH broth served as negative control, another well with the test strain only was considered as positive control. For screening against Mycobacterium bovis bacillus Calmette-Guerin (BCG), BCG expressing luxAB was diluted in Middlebrook 7H9 medium (106 CFU; 50 μl/well) in 96-well opaque white plates (Corning).

Fifty microliters of the clarified cell lysate were added to the wells, the plates were incubated at 37° C. for 24 h before adding 0.1% n-decyl aldehyde (Decanal, Sigma), a substrate for bacterial luciferase. Bioluminescence was measured as relative luminescence unit (RLU) for 1 s using a TriStar2 microplate reader (Berthold Technologies) (Tenland et al., 2018).

### Results

### Library Construction

The correct assembly of the libraries was checked through sequencing (GATC Biotech AB, Solna, Sweden) of the shuffled enzymes and peptides each on its correct position. Sequencing analysis of the sequenced clones was done using Geneious prime software, (Geneious version 2020.1.2; Biomatters Ltd., Auckland, New Zealand).

### Expression of the Constructed Libraries

The expression levels of the expressed libraries were checked on SDS-PAGE. The expression levels of the initial expression screening of the constructed libraries were quite low, no protein bands corresponding to the estimated molecular mass of the expressed clones were detected on SDS-PAGE (FIG. 2). On the other hand, expression in LB medium resulted in decent expression levels corresponding to the calculated molecular mass both at 30° C. and 16° C., however the majority of the expressed proteins are in the insoluble fraction (FIG. 2).

### Screening of the Expressed Libraries

The initial screening of most the constructed libraries was done against Mycobacterium smegmatis mc² 155 strain with a bacterial load of (≈4×10⁵ CFU/ml) and resulted in many active hits (Data not shown). The test stringency was increased to make a filtration/selection criterion through gradual increase of the bacterial load from (≈4×10⁵ up to 4×10⁸ CFU/ml). The data was normalized by subtraction from the negative control well.

Wells with no visible bacterial growth both at the end of the incubation period and after subculture on LB agar plates were considered as positive antibacterial activity hits. Wells with bacterial growth were compared with the positive control well for identification of any inhibitory effect of the expressed clones in the corresponding wells.

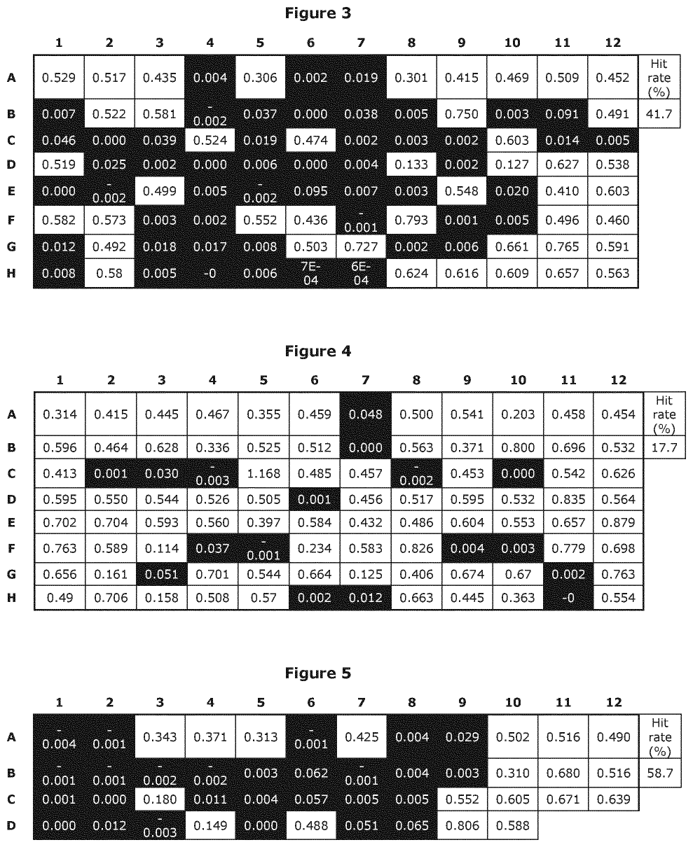

Library 1 with the organization (AMPs-LysB-Linker-LysA) exhibited the highest numbers of positive active hits against Mycobacterium smegmatis mc² 155 strain with a bacterial load of (≈4×10⁸ CFU/ml) (FIG. 3).

Screening of the second library against Mycobacterium smegmatis mc² 155 strain with a bacterial load of (≈4×10⁸ CFU/ml) resulted in much lower number of active variants (FIG. 4), potentially due to a lack of the cell wall binding domains (CBD) that are present in LysA enzymes shuffled in the first library. The proposed function of the cell wall binding domain is to bring the catalytic domain to a proximity of the peptidoglycan thus facilitating its interaction with the peptidoglycan layer of the mycobacterial cell wall. Moreover, LysA enzymes are modular enzymes comprising more than one catalytic domain with different mechanisms of action targeting different bonds in the peptidoglycan structure and a cell wall binding domain.

Due to the limited transformation efficiency of the third library, only few clones were transformed and screened against Mycobacterium smegmatis mc² 155 strain (≈4×10⁸ CFU/ml) with some active hits were obtained (FIG. 5).

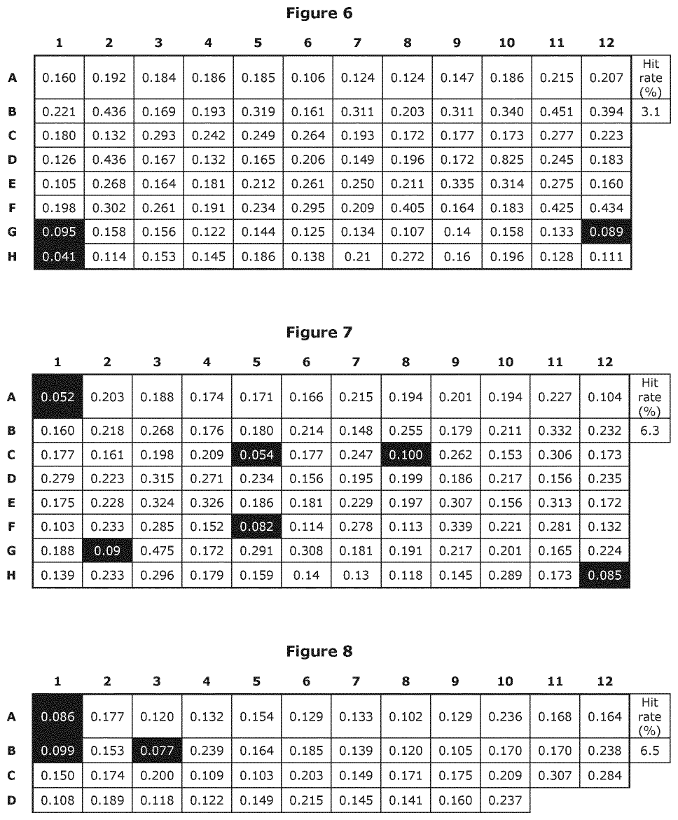

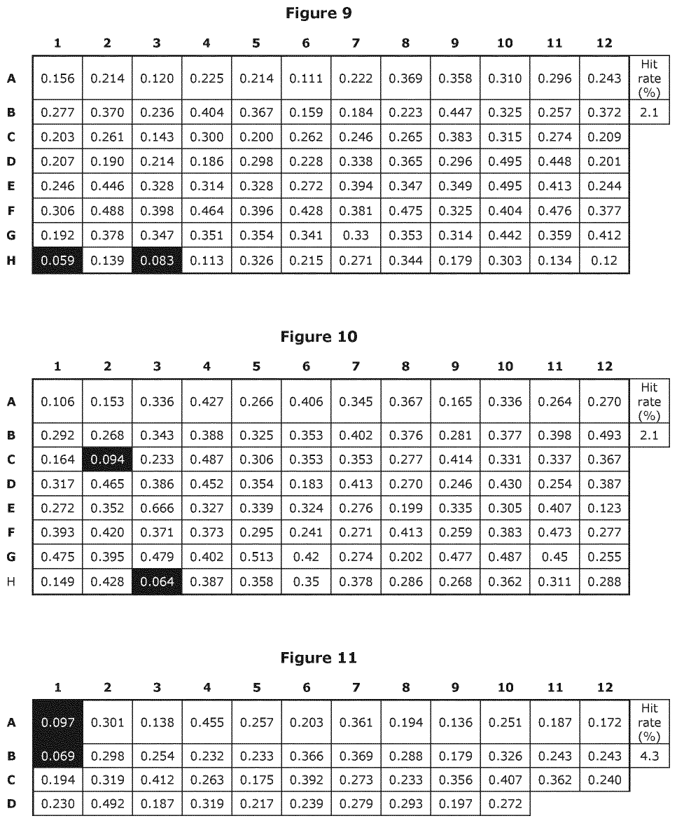

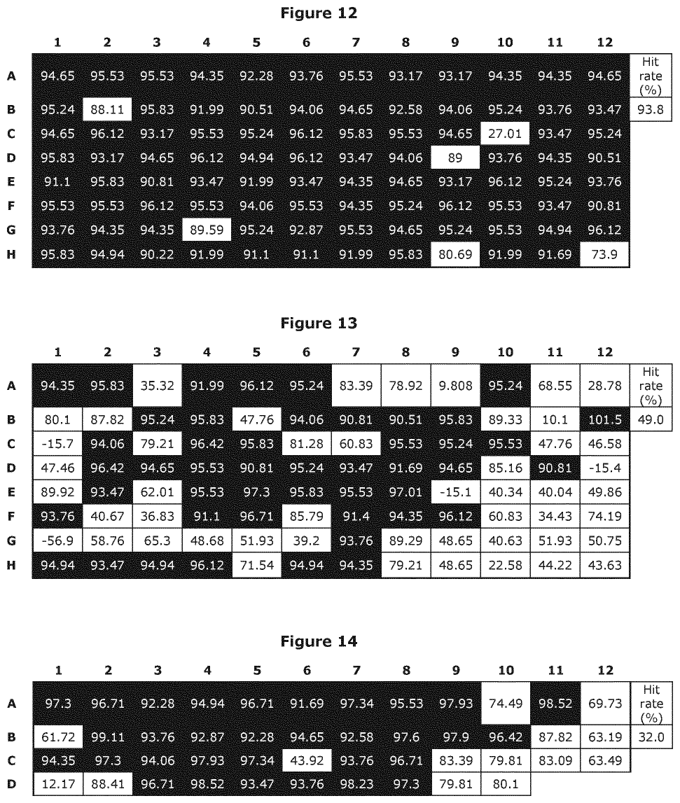

The expressed libraries (1, 2 and 3) were also tested for their antibacterial activities against different species from the order Actinomycetales including Nocardia iowensis (FIGS. 6-8) and Rhodococcus erythropolis (FIGS. 9-11) and resulted in some clones with antibacterial activity. The expressed libraries were also tested for their antibacterial activity against some pathogenic strains including Mycobacterium bovis bacillus Calmette-Guerin (BCG) and Mycobacterium abscessus strains. The antibacterial activity against Mycobacterium bovis bacillus Calmette-Guerin (BCG) was expressed as percentage of growth inhibition of the bacterial cells compared to positive control (FIGS. 12-14). The majority of the expressed clones from libraries 1, 2 and 3 showed a potential antibacterial activity against BCG strain with % antibacterial efficiency up to 98% (FIGS. 12-14).

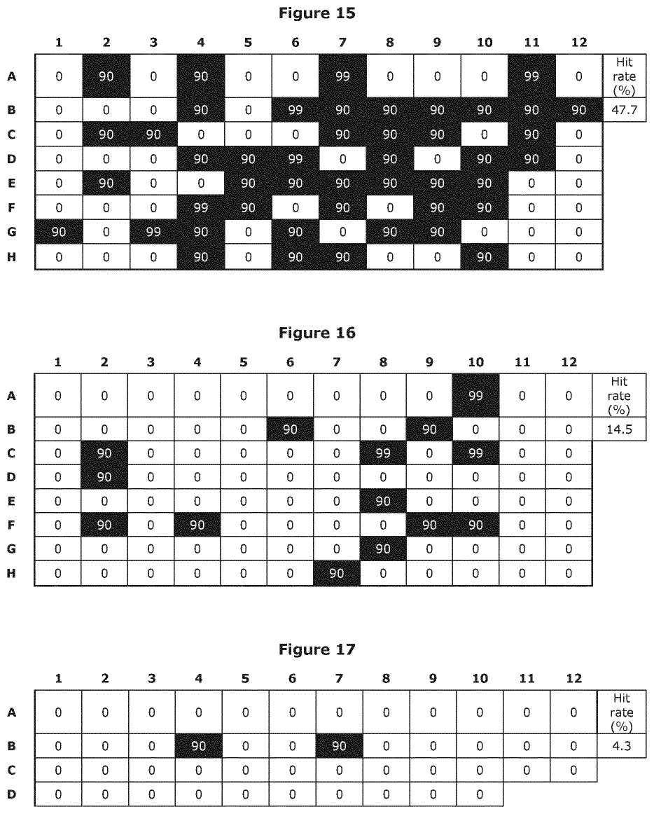

On the other hand, the antibacterial activity against the pathogenic Mycobacterium abscessus was expressed as 99, 90 and 0% inhibition in comparison with positive control (FIGS. 15-17). Almost half of the expressed clones from the library 1 showed antibacterial inhibition against Mycobacterium abscessus with 6 clones demonstrated 99% inhibition (FIG. 15). Moreover, only 14 clones expressed from library 2 were positive against Mycobacterium abscessus with 3 variants showed 99% inhibition (FIG. 16). On the contrary, only 2 variants expressed from library 3 inhibited the growth of Mycobacterium abscessus by 95% (FIG. 17).

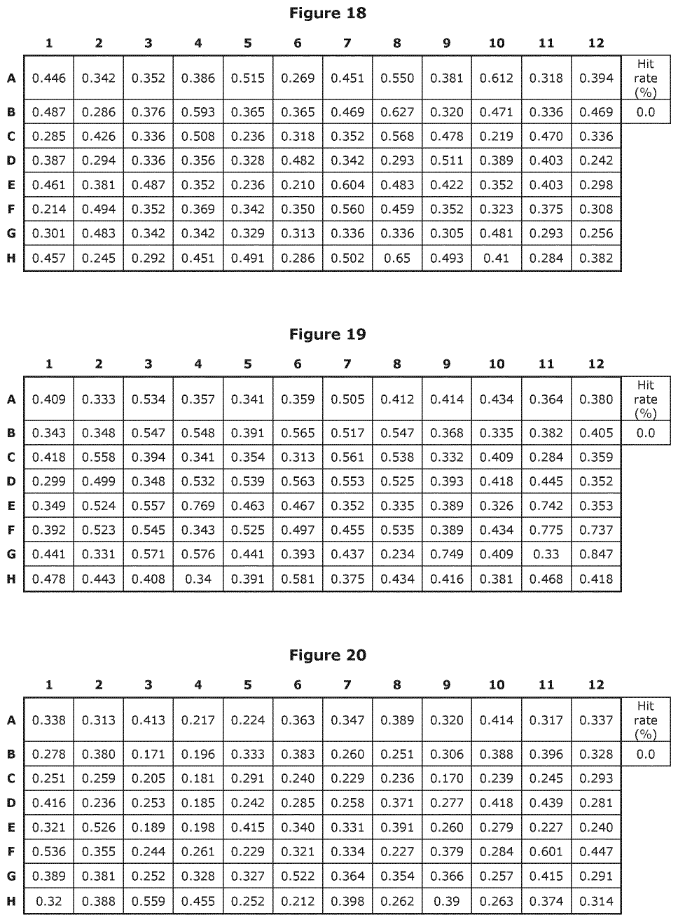

For exclusion criteria, different libraries with domain organizations were constructed and screened. Expressed clones from library 4 (AMPs-LysB) did not exhibit antimycobacterial activity against Mycobacterium smegmatis (FIG. 18).

Addition of linkers between AMPs and LysB enzymes was used to construct library 5 (AMPs-Linkers-LysB), which did not exhibit antimycobacterial activity after expression against Mycobacterium smegmatis (FIG. 19).

Library 6 was constructed by direct fusion of AMPs with LysA enzymes and failed to express antimycobacterial activity against Mycobacterium smegmatis as well (FIG. 20).

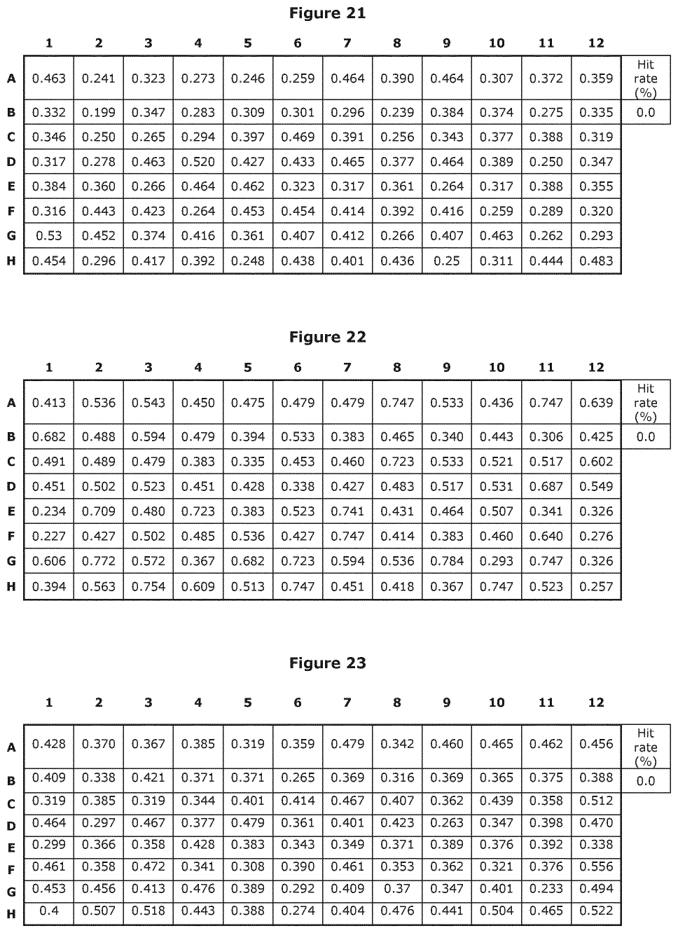

Separating the AMPs from LysA enzymes by linkers was the basis to design library 7 which upon screening against Mycobacterium smegmatis did not show antimycobacterial activity (FIG. 21). The enzyme active domains (EADs) were either fused directly to AMPS (library 8) or via linkers (library 9) which did not result in antimycobacterial activity upon testing against Mycobacterium smegmatis (FIGS. 22 and 23).

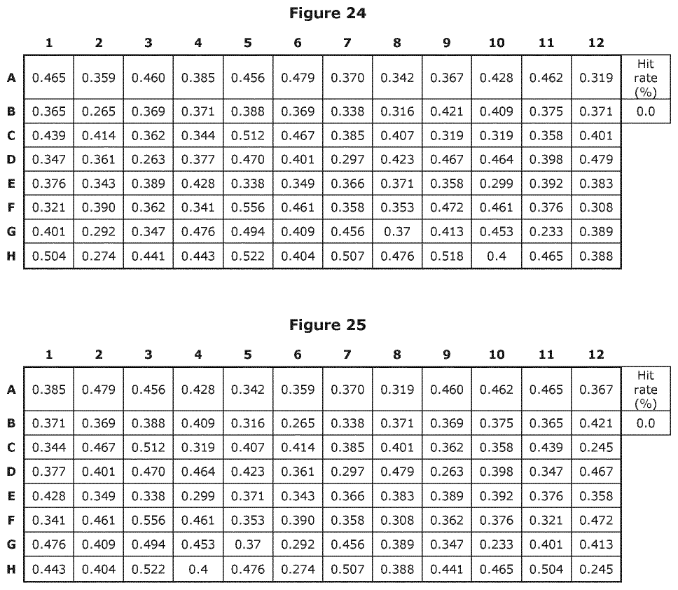

Furthermore, different libraries had been constructed through excluding the AMPs, LysB enzymes were shuffled with either LysA enzymes (library 10) or EADs (library 11). It seems that the AMPs are crucial for exerting the antibacterial activity, since there was no antibacterial activity was detected for libraries 10 and 11 against Mycobacterium smegmatis with a bacterial load of 10⁵ CFU/ml (FIGS. 24 and 25).

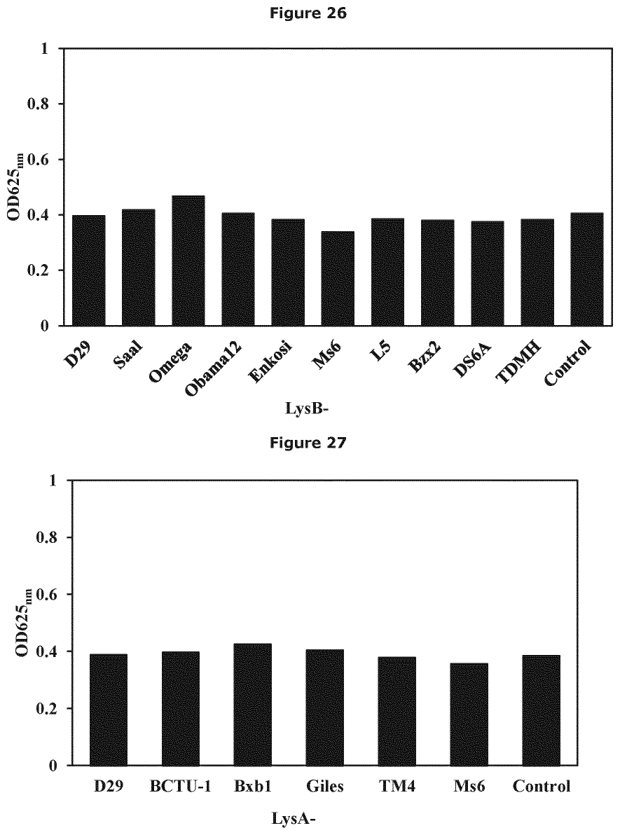

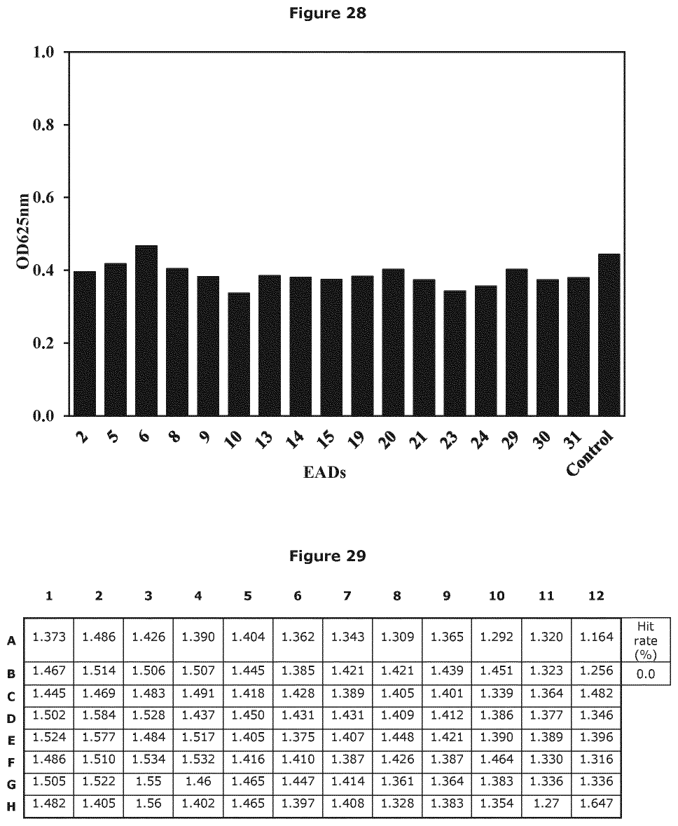

In addition, LysB, LysA enzymes as well as EADs were cloned individually and expressed in E. coli codon plus expression host and their antibacterial activity was also tested. None of the respective enzymes exhibited antibacterial activity against Mycobacterium smegmatis with a bacterial load of 10⁵ CFU/ml (FIGS. 26-28) suggesting that the antibacterial activity is a result of synergistic effect of LysB, LysA/EADs and AMPS.

AMPs that are active against gram negative or Gram-positive bacteria (which may have no activity when tested against mycobacteria) can be combined with LysB and LysA/EAD to result in peptides capable of activity against Mycobacterium. Alternatively, AMPs with activity specific to mycobacteria may be used.

Peptides resembling those of US 2017/0136102 A1 (Sharma) failed to demonstrate activity against Mycobacterium, even in the presence of anti-TB drugs. The Sharma peptides are comprised of an AMP with LysB but lack any LysA (or LysA-like domain/EAD), and so it is not unexpected that such peptides actually fail to produce antimycobacterial activity.

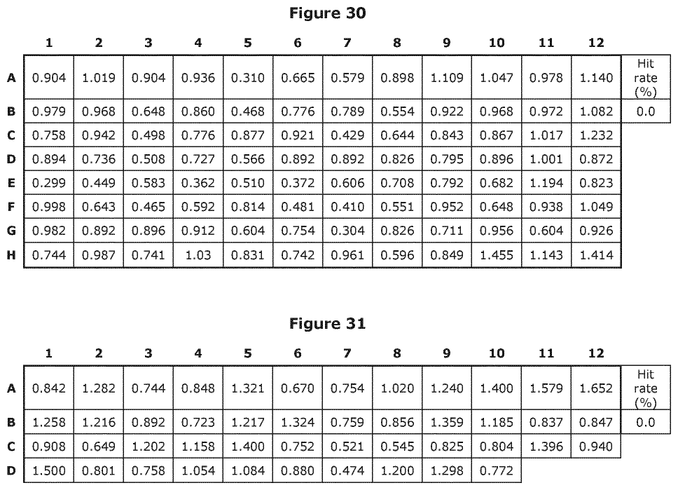

The expressed libraries (1, 2 and 3) were tested for their antibacterial selectivity/specificity through testing against Gram-positive and negative strains. A representative of the pathogenic Gram-positive tested bacterial strain is Staphylococcus aureus, against which none of the expressed clones from the corresponding libraries (1, 2 and 3) showed bacterial growth inhibition (Data not shown). In the same context, the expressed libraries (1, 2 and 3) did not exhibit bactericidal activity against the Gram-negative tested E. coli strain (FIGS. 29-31). Altogether, these data indicate that the specificity/selectivity of the expressed libraries (1, 2 and 3) is against mycobacterial strains only.

TABLE 4
Constructs showing antimycobacterial activity

| | | | | | | Anti- |
|---|---|---|---|---|---|---|
| | | | | | | mycobacterial |
| Library | Pos1 | Pos2 | Pos3 | Pos4 | Pos5 | activity |
| 1 | AMPs | LysB | Linker | LysA | — | YES |
| 2 | AMPs | LysB | Linker | EADs | | YES |
| 3 | AMPs | LysB | Linker | LysA | PTDs | YES |
| 4 | AMPs | LysB | — | — | — | NO |
| 5 | AMPs | Linker | LysB | — | — | NO |
| 6 | AMPs | LysA | — | — | — | NO |
| 7 | AMPs | Linker | LysA | — | — | NO |
| 8 | AMPs | EADs | — | — | — | NO |
| 9 | AMPs | Linker | EADs | — | — | NO |
| 10 | LysB | Linker | LysA | — | — | NO |
| 11 | LysB | Linker | EADs | — | — | NO |

### CONCLUSION

For all libraries prepared, only a small fraction of the possible combinations has been tested as a proof in principle for workable fusions of particular configurations. Therefore, the exemplified hits in libraries should not be construed as being the only hits that work for a particular library. The key point is that the proof in principle data shows that workable constructs can be developed of certain configurations (i.e. those that contain (i) a first domain with activity specific to a peptidoglycan or component thereof, (ii) a second domain with activity specific to an ester linkage; and (iii) a third domain with membrane permeabilising activity), while configurations lacking these essential components always fail to demonstrate antimycobacterial activity unless they are used in particular mixtures.

### Example 2

After observing the positive findings for Libraries 1-3, additional libraries were constructed in which the domains present, and their positioning within the fusion protein, were altered (see Table 5). The same screening approach was performed as with Example 1.

TABLE 5
Additional constructs showing antimycobacterial activity

| | | | | | | Anti- |
|---|---|---|---|---|---|---|
| | | | | | | mycobacterial |
| Library | Pos1 | Pos2 | Pos3 | Pos4 | Pos5 | activity |
| 12 | AMPs | LysA | Linker | LysB | — | YES |
| 13 | AMPs | EAD | Linker | LysB | — | YES |
| 14 | LysB | Linker | LysA | AMP | — | YES |
| 15 | LysA | Linker | LysB | AMP | — | YES |
| 16 | LysB | AMP | LysA | — | — | YES |

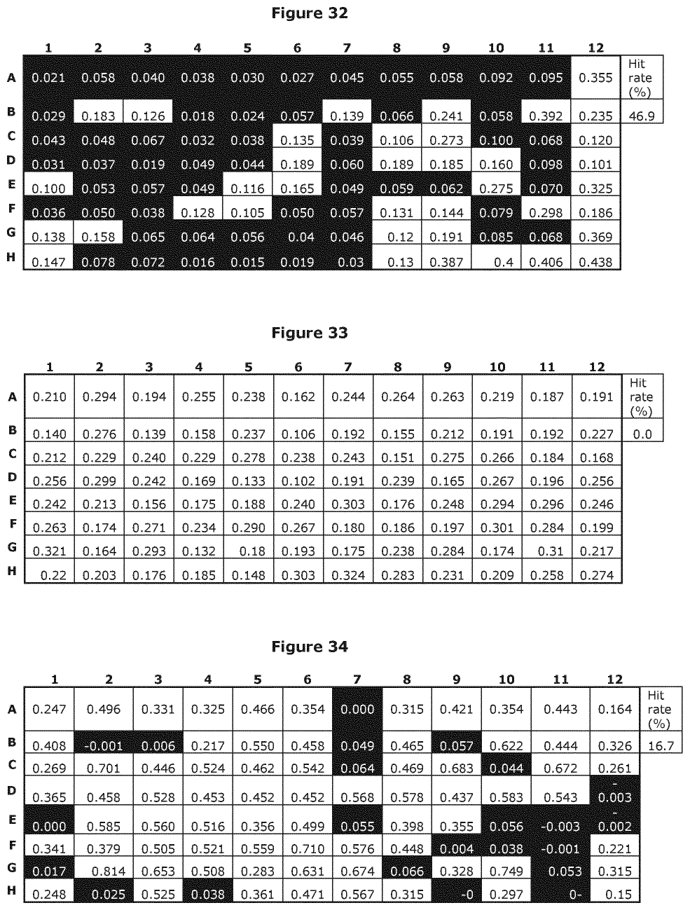

Library 12 with the organization (AMPs-LysA-Linker-LysB) exhibited the highest numbers of positive active hits against Mycobacterium smegmatis mc² 155 strain with a bacterial load of (≈4×10⁸ CFU/ml) (FIG. 32), at a slightly higher rate than was seen for Library 1.

Library 13 resulted in a much lower number of active variants (FIG. 34), potentially due to a lack of the cell wall binding domains (CBD) that are present in LysA enzymes shuffled in the first library, as hypothesised previously with respect to Libraries 1 and 2.

The domains of Library 12 were reversed to form Library 14. This means that the orientation of each domain is flipped, such that the previous N- to C-terminal fusions of Library 12 are instead in a C- to N-configuration for Library 14. For example, an exemplary peptide of Library 12 may be comprised of SEQ ID NOs: 58+12+108+16, from N-terminus to C-terminus, meaning that the C-terminus of SEQ ID NO: 58 is fused to the N-terminus of SEQ ID NO: 12, and so on. The reversed peptide of Library 14 would therefore be SEQ ID NOs: 16+108+12+58, from N-terminus to C-terminus, meaning that the N-terminus of SEQ ID NO: 58 is fused to the C-terminus of SEQ ID NO: 12, and so on.

Library 12 exhibited a hit rate of 46.9%, and reversing the orientation of the domains maintained a hit rate of 36.5%.

The domains of Library 1 were also reversed to form Library 15 (in the same way as Libraries 12 and 14 with respect to each other). However, despite reorientation of the domains in the fusion protein, a number of successful hits for antimycobacterial fusion peptides were identified.

These reorientation data demonstrate that the domains of the fusion peptide can be in either orientation with respect to each other, while maintaining the beneficial properties of the fusion peptides.

A further library (library 16) was developed that used AMP domains in place of a linker between LysB and LysA (i.e. LysB-AMP-LysA fusion peptides), to form 3-domain fusion peptides that exclude the linkers described herein. A number of successful hits for antimycobacterial fusion peptides were identified, thereby demonstrating that a linker is not essential for retaining the beneficial properties of the fusion peptides. An alternative way to consider library 16 is that the AMP acts as a linker between LysA and LysB.

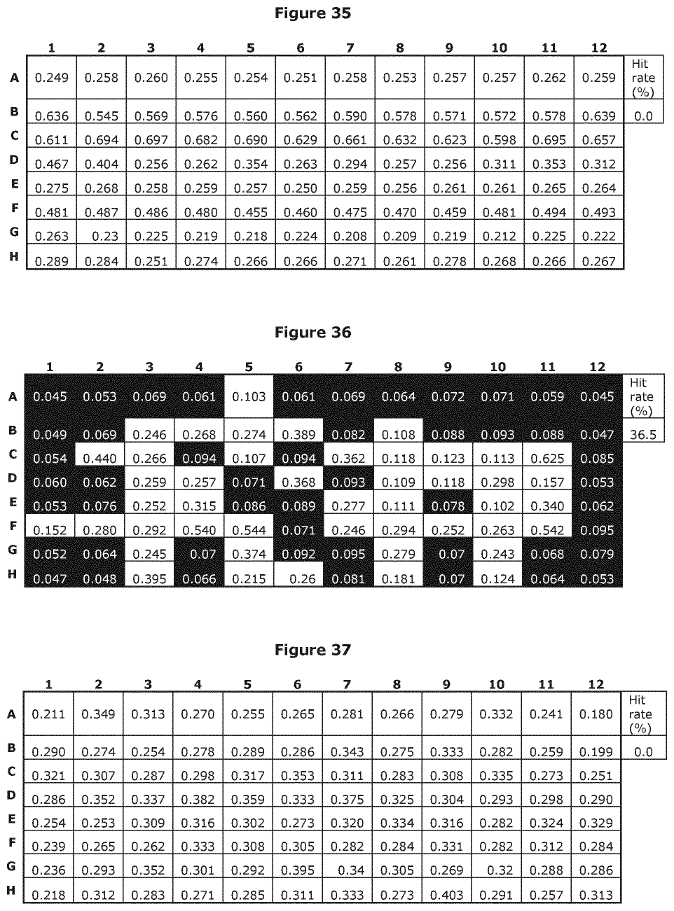

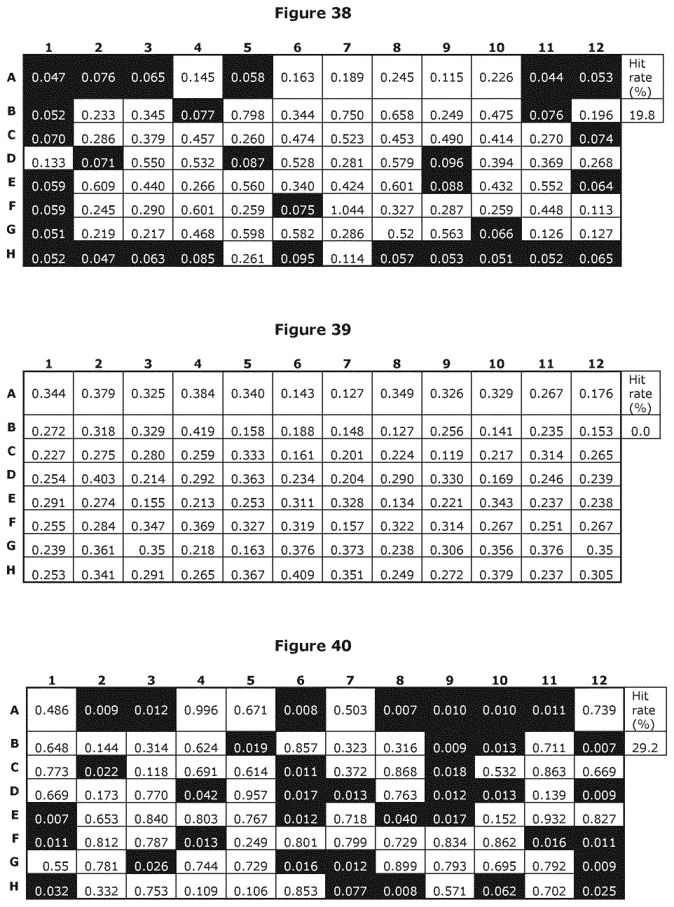

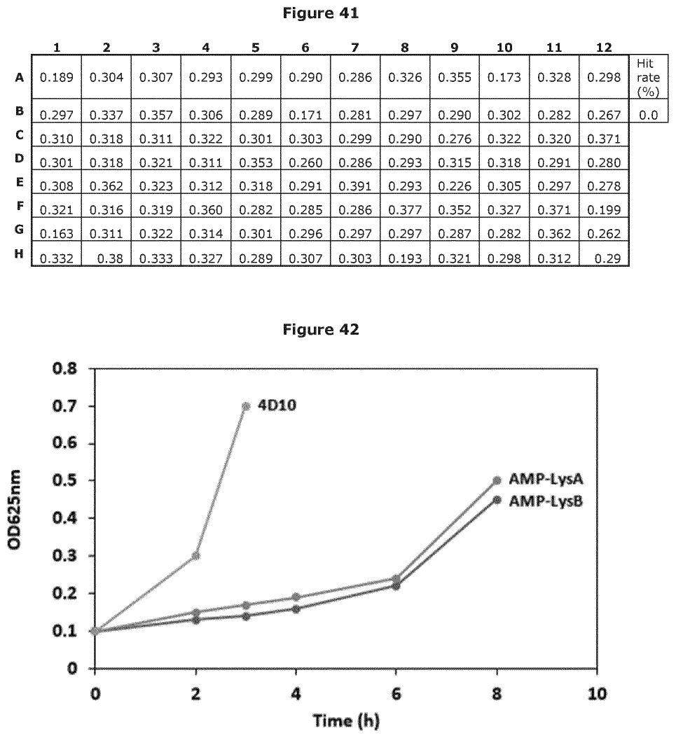

As previously shown for libraries 1-3, in the same context, the expressed libraries (12-16) did not exhibit bactericidal activity against the Gram-negative tested E. coli strain (FIGS. 33, 35, 37, 39 and 41). Altogether, these data indicate that the specificity/selectivity of the expressed libraries (12-16) is against mycobacterial strains only.

### CONCLUSION

As with Example 1, for all libraries prepared, only a small fraction of the possible combinations and/or reorientations of domains have been tested as a proof in principle for workable fusions of particular configurations. Therefore, the exemplified hits in libraries should not be construed as being the only hits that work for a particular library. The key point is that the proof in principle data shows that workable constructs can be developed of certain configurations (i.e. those that contain (i) a first domain with activity specific to a peptidoglycan or component thereof, (ii) a second domain with activity specific to an ester linkage; and (iii) a third domain with membrane permeabilising activity), while configurations lacking these essential components always fail to demonstrate antimycobacterial activity unless they are used in particular mixtures, and that the domains used can be oriented in either direction within the fusion peptide.

### Example 3

Growth curves were prepared when constructing the various libraries to assess whether there were any differences in expressing the proteins based on their configuration. The comparison was made based on optical density (OD_{625 nm}), which positively correlates with expression levels (i.e. a higher mass of bacterial growth results in a larger expression level of the fusion protein).

The components used for all conditions were as follows:
AMP=Ci-MAM-A24;
Linker=Linker 1;
LysA=TM4; and
LysB=D29.

Fusions of AMP-LysA, AMP-LysB and AMP-LysB-Linker-LysA (referred to as 4D10) were created using these domains, and expressed in bacteria as previously described. The bacteria were grown in LB medium for up to 8 hours, with optical density being assessed at 2, 3, 4, 6 and 8 hours. For 4D10, a considerably faster expression level was achieved, as shown by the vastly increased optical density at 2 and 3 hours in FIG. 42. Surprisingly, even 8 hours later the AMP-LysA and AMP-LysB conditions did not achieve equivalent expression levels as 4D10 at 3 hours.

These data support an advantage of the fusion peptides comprised of all three domains (compared with the prior art 2-domain peptides), which are able to be expressed faster. It is also important to note the advantage of expressing the required proteins for antimycobacterial activity in a single bacterial colony. On the other hand, if mixtures of AMP-LysA and AMP-LysB are required to achieve antimycobacterial activity, then two separate bacterial colonies must be created, each protein purified, and the mixture then formed. The present invention therefore streamlines the process while obtaining the surprising technical effect of faster expression. Furthermore, AMP-LysA and AMP-LysB constructs were observed to be toxic to E. coli, used as the host for peptide expression, thereby resulting in a lower rate of expression. Surprisingly, fusion peptides comprising AMP, LysA and LysB removed this host toxicity, resulting in the faster rate of expression. Accordingly, by creating fusions of AMP-LysA-LysB, such as 4D10, it is possible to express anti-mycobacterial peptides in host E. coli that would otherwise (i.e. in an AMP-LysA or AMP-LysB format) result in E. coli cytotoxicity.

A summary of the growth inhibition and mycobacterial specificity for clone 4D10 can be seen in the Table 6.

TABLE 6
Growth inhibition of various mycobacteria
and bacterial controls for 4D10.

| Test strain | Growth inhibition |
|---|---|
| Mycobacterium smegmatis 10⁸ CFU/ml | >70% |
| Mycobacterium bovis 10⁶ CFU/ml | >99% |
| Mycobacterium abscessus 10⁷ CFU/ml | >99% |
| E. coli 10⁸ CFU/ml | 0% |
| Staphylococcus aureus 10⁸ CFU/ml | 0% |
| Micrococcus lysodeikticus 10⁶ CFU/ml | 0% |

### Example 4

### Methods

### Expression and Purification

Different combinations and mixtures of AMP (Ci-MAM-20, SEQ ID NO: 37), LysA (TM4, SEQ ID NO: 13) and LysB (D29, SEQ ID NO: 15) were constructed according to Table 7. Each construct was expressed in E. coli CodonPlus (DE3)-RIL, preculture of 10 mL of LB medium supplemented with kanamycin and grown overnight. 150 ml of Terrific Broth (TB) medium supplemented with Kanamycin were inoculated with 3 ml of the preculture, and incubated at 37° C. When the optical density (OD600ₙₘ) reached 0.5-0.6, the cells were induced for protein expression using IPTG at a final concentration of 0.5 mM at 16° C. for 18 hours. The cells were harvested by centrifugation and the cell pellet was resuspended in 20 ml of binding buffer (50 mM Tris-HCl, 0.5 M NaCl, 20 mM imidazole, pH 8) and sonicated on ice. Lysates were cleared by centrifugation (18,500×g, 30 min) and filtered (0.22-μm). The clarified lysate was purified using HisTrap FF™ nickel column (GE Healthcare Bio-Sciences AB, Uppsala, Sweden). The eluted fractions were pooled together and dialyzed against dialysis buffer (50 mM Tris-HCl, 0.5 M NaCl, pH 8) overnight at 4° C. Protein concentration was measured by nanodrop, and samples were kept at 4° C.

### Testing the Antibacterial Activity of the Purified Samples

Mycobacterium smegmatis mc² (155; ATCC 700084) was grown overnight in Mueller-Hinton broth (MHB) at 37° C. For the assay, 50 μl of 4×10⁵ CFU/ml M. smegmatis cells were mixed with 50 μl of the purified proteins (35-50 μg) in a microtiter plate in duplicates and incubated at 37° C. for 24 hours. The endpoint measurement at OD625ₙₘ was performed in a microplate reader (Multiskan™ GO Microplate Spectrophotometer, Thermo Scientific) after 24 hours. A well with 100 μl MHB served as 100% inhibitory activity positive control, and another well with M. smegmatis cells was considered as 0% inhibitory activity negative control.

### Results

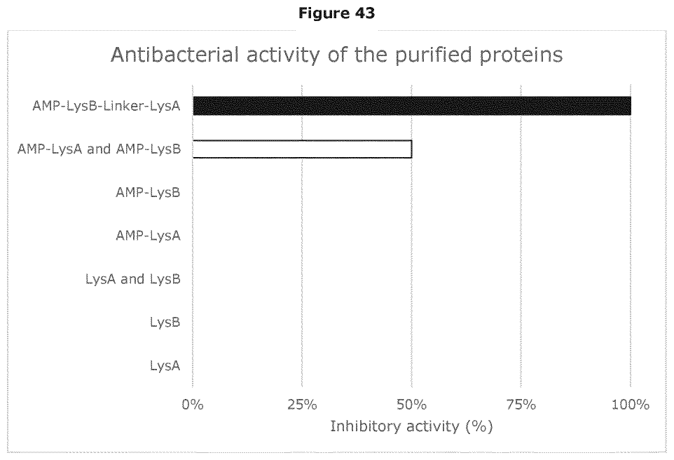

Neither LysA (50 μg) nor LysB (50 μg) alone or in a mixture showed any antimycobacterial activity against M. smegmatis. Neither AMP-LysA (50 μg) nor AMP-LysB (50 μg) alone showed any antibacterial activity, but when both were present in a mixture (AMP-LysA (50 μg) and AMP-LysB (50 μg)), 50% inhibitory activity was observed (FIG. 43). Surprisingly, however, the fusion protein AMP-LysB-Linker-LysA (Seq ID: 38+16+104+14) 35 μg showed 100% inhibitory activity (FIG. 43).

TABLE 7
The antibacterial activity of the purified proteins

| | SEQ | Total amount | Concentration | Inhibitory |
|---|---|---|---|---|
| Construct/Mixture | ID NOS | (μg) | (μM) | activity (%) |
| LysA | 14 | 50 | 8.4 | 0 |
| LysB | 16 | 50 | 17 | 0 |
| LysA and | 14 and 16 | 50 + | 25.4 | 0 |
| LysB | | 50 = 100 | | |
| AMP-LysA | 38 + 14 | 50 | 7.8 | 0 |
| AMP-LysB | 38 + 16 | 50 | 14.6 | 0 |
| AMP-LysA and | (38 + 14) and | 50 + | 22.2 | 50 |
| AMP-LysB | (38 + 16) | 50 = 100 | | |
| AMP-LysB- | 38 + 16 + | 35 | 3.75 | 100 |
| Linker-LysA | 104 + 14 | | | |

There are potential safety issues for using certain AMPs at high doses, and so it is advantageous to be able to use AMPs at lower doses while maintaining AMP activity. Therefore, the amount of each component was estimated using a percentage fraction based on kDa of the fusion peptide (see Table 8). These data demonstrate that the fusion peptide is more effective despite being at a lower over amount (in μg), which allows the creation of more effective fusion peptides with increased function at lower concentrations. Such improved peptides are particularly advantageous from a safety point of view for dosing recipients of the fusion peptides.

TABLE 8
Amount and concentration of each component used based on kDa.

| | Mixture | | Fusion peptide | |
|---|---|---|---|---|
| | AMP-LysA and AMP-LysB | | AMP-LysB-Linker-LysA | |
| Element | μg | μM | μg | μM |
| LysA | 47.7 | 7.8 | 22.0 | 3.8 |
| LysB | 45.5 | 14.6 | 10.7 | 3.8 |
| AMP | 6.8 | 22.3 | 1.1 | 3.9 |
| Fusion/ | 100 | NA | 35 | 3.8 |
| Mixture | | | | |

The concentration of AMP, LysA and LysB is substantially lower in the fusion peptide AMP-LysB-Linker-LysA, and AMP in particular with almost 6 times lower concentration than used in the mixture (AMP-LysA and AMP-LysB), which only obtained half the inhibitory activity of the fusion peptide.

1. A peptide comprising:
(i) a first domain with activity specific to a peptidoglycan or component thereof, and
(ii) a second domain with activity specific to an ester linkage; and
(iii) a third domain with membrane permeabilising activity.

2. The peptide according to claim 1, further comprising a fourth domain that is at least one protein transduction domain (PTD).

3. The peptide according to claim 1, wherein the first domain is an enzyme domain.

4. The peptide according to claim 1, wherein the second domain:
(iv) is an enzyme domain;
(v) is specific to the ester linkage between mycolic acid and arabinogalactan; or between mycolic acid and trehalose or between arabinogalactan and peptidoglycan, and/or any member of the alpha/beta hydrolase family;
(vi) is specific to alpha and/or beta hydrolases;
(vii) is a mycolyl arabinogalactan esterase and/or a mycolyl-arabinogalactan-(vii) peptidoglycan (mAGP) hydrolase; and/or
(viii) have mechanisms of action corresponding to alpha/beta hydrolase activity, esterase activity, lipase activity, protease activity, TDMH, cutinase activity, trehalose dimycolate hydrolase (TDMH), Pectinesterase, CheB methylesterase, Glycerophosphoryl diester phosphodiesterase, Plant invertase/pectin methylesterase inhibitor, Carboxylesterase family, Calcineurin-like phosphoesterase, Putative esterase, Thioesterase domain, Hemagglutinin esterase, Calcineurin-like phosphoesterase superfamily domain, Pectinacetylesterase, Putative serine esterase, Esterase PHB depolymerase, Esterase-like activity of phytase, Chitin recognition protein, Glycosyl hydrolase all families, Amidase, Lipase all families, GDSL-like Lipase/Acylhydrolase, Partial alpha/beta-hydrolase lipase region, GDSL-like Lipase/Acylhydrolase family, Secretory lipase, Patatin-like phospholipase, Carboxylesterase, Variant-surface-glycoprotein phospholipases all families, Putative lysophospholipase, Alpha/beta-hydrolase superfamily, Hydrolase, haloacid dehalogenase-like hydrolase, epoxide hydrolase and dehalogenases, peroxidase, or any combinations thereof.

5. (canceled)

6. The peptide according to claim 1, wherein the peptide comprises multiple domains corresponding to a first domain, second domain, third domain, and/or fourth domain, wherein the multiple domains are:
(i) repeats of the same domain;
(ii) different domains but with the same type of activity;
(iii) derived from the same source; or
(iv) derived from different sources.

7. The peptide according to claim 1, wherein the third domain with membrane permeabilising activity is an antimicrobial peptide (AMP) or portion thereof, a holin or portion thereof, and/or a spanin or portion thereof,
optionally wherein the AMP or a portion thereof is cationic, polycationic, hydrophobic, amphipathic, synthetic or natural or any combination thereof (or comprises portions that are cationic, polycationic, hydrophobic, amphipathic, synthetic or natural, or any combination thereof).

8. The peptide according to claim 1, wherein the peptide further comprises at least one linker peptide, wherein the linker peptide connects:
(i) the first domain to the second domain;
(ii) the second domain to the third domain;
(iii) the first domain to the third domain; and/or
(iv) a domain to a further linker.

9. The peptide according to claim 1, wherein:
(i) the first domain and/or second domain comprises a domain with a mechanism of action selected from the group consisting of amidase, transglycosylase, chitinase, muramidase and/or peptidase, N-acetylmuramoyl-L-alanine amidase, Amidases in general including all families, D-Alanine-meso-Diaminopimelic (DD) endopeptidase, c-D-glutamyl-meso-diaminopimelic acid (DL) peptidase, Endopeptidases in general including all families, lytic trans glycosylases, N-acetylmuramidase, lysozyme, L-alanoyl-D-glutamate (LD), m-DAP-m-DAP (LD) endopeptidase in general, D-alanyl-D-alanine carboxypeptidase, Glycoside hydrolases in general including all families, L-Alanine-D-Glutamate peptidase, cysteine protease, N-acetyl-β-D-glucosaminidase carboxypeptidase, glycosidase (glucosaminidases), transpeptidases, epimerase, γ-D-glutamyl-meso-diaminopimelic acid (DL) peptidases, and/or combinations thereof;
(ii) any one or more of the domains are effective against the phylum Actinobacteria; derived from the class Actinobacteridae; derived from the order Actinomycetales, and/or Bifidobacteriales;
(iii) any one or more of the domains are effective against Mycobacterium selected from the group consisting of: Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abscessus, Mycobacterium kansasii, Mycobacterium terrae, Mycobacterium nonchromogenicum, Mycobacterium gordonae, and Mycobacterium triviale, and non-tuberculosis mycobacteria (NTM); and/or
(iv) any one or more of the domains are derived from at least one mycobacteriophage and/or at least one Mycobacterium prophage.

10. The peptide according to claim 1, wherein any one or more of the domains are a mutant, variant or wildtype domain;
wherein the mutant or variant retains substantially the same level of activity of the wildtype domain.

11-13. (canceled)

14. A polynucleotide, vector, and/or cell comprising a nucleic acid encoding a peptide comprising
(i) a first domain with activity specific to a peptidoglycan or component thereof, and
(ii) a second domain with activity specific to an ester linkage; and
(iii) a third domain with membrane permeabilising activity.

15. (canceled)

16. An in vitro method of lysing, killing, reducing growth of, or reducing viability of mycobacteria, wherein the method comprises administering a peptide according to claim 1.

17. A device comprising a peptide according to claim 1.

18.-19. (canceled)

20. The peptide according to claim 3, wherein the enzyme domain is a lysin.

21. The peptide according to claim 1, wherein the first domain is a LysA or at least a portion thereof, or the second domain is a LysB or at least a portion thereof.

22. The peptide according to claim 21, wherein:
(i) the portion of LysA or the portion of LysB is an enzyme active domain (EAD); or
(ii) the LysA or LysB comprises a cell wall binding domain (CBD).

23. The peptide according to claim 22, wherein the cell wall binding domain (CBD) is:
(i) specific to a bacterial target of interest;
(ii) is not specific to the cell walls of Gram positive or Gram negative bacteria; or
(iii) is specific to mycobacterial cell walls.

24. A method for treating infection, granulomas or biofilms, or promoting wound healing, the method comprising administering to an individual in need thereof a therapeutically effective amount of a peptide comprising:
(i) a first domain with activity specific to a peptidoglycan or component thereof, and
(ii) a second domain with activity specific to an ester linkage; and
(iii) a third domain with membrane permeabilising activity.

25. The method according to claim 24, wherein the infection is:
(i) a mycobacterial infection; or
(ii) tuberculosis; or
(iii) an infection caused by a bacteria selected from Mycobacterium tuberculosis, Mycobacterium microti, Mycobacterium africanum, Mycobacterium bovis, Mycobacterium canettii, Mycobacterium pinnipedii, Mycobacterium caprae, Mycobacterium mungi, Mycobacterium leprae, Mycobacterium ulcerans, Mycobacterium xenopi, Mycobacterium shottsii, Mycobacterium avium, Mycobacterium avium subsp. paratuberculosis, Mycobacterium paratuberculosis, Mycobacterium intracellulare, Mycobacterium smegmatis, Mycobacterium abscessus, Mycobacterium kansasii, Mycobacterium terrae, Mycobacterium nonchromogenicum, Mycobacterium gordonae, and Mycobacterium triviale, and non-tuberculosis mycobacteria.
(iv) a respiratory, ocular, skin, oral or dental infection; or
(v) a bacterial infection caused by a bacterium that is resistant to at least one drug or is classified as multidrug-resistant (MDR), extremely drug-resistant (XDR), or totally drug-resistant (TDR).

26. The method according to claim 24, further comprising administration of at least one additional therapeutic agent.

27. The method according to claim 26, wherein the additional therapeutic agent is an anti-mycobacterial agent.
